(19) United States
(12) Patent Application Publication
(10) Pub. No.: US 2021/0000756 A1
(43) Pub. Date: Jan. 7, 2021

(54) TRANSDERMAL THERAPEUTIC SYSTEM COMPRISING A SILICONE ACRYLIC HYBRID POLYMER

(71) Applicant: LTS LOHMANN THERAPIE-SYSTEME AG, Andemach (DE)

(72) Inventors: Marco EMGENBROICH, Rheinbach (DE); Gabriel WAUER, Bad Neuenahr-Ahrweiler (DE); Michael LINN, Waldbockelheim (DE); Rolf BÖHM, Kleinmaischeid (DE); Christoph SCHMITZ, Rheinbrohl (DE); Regine KAUFMANN, Neuwied (DE); Hans-Werner WOLF, Neuwied (DE); Nico REUM, Mendig (DE); Anna SCHLÜTER, Bonn (DE)

(21) Appl. No.: 16/979,575

(22) PCT Filed: Mar. 11, 2019
(86) PCT No.: PCT/EP2019/056016

§ 371 Date: Sep. 10, 2020

(30) Foreign Application Priority Data
Mar. 13, 2018 (EP) 18161432.2

(51) Int. Cl. A61K 9/70; A61K 31/485; A61K 31/465; A61K 31/27; A61K 47/32; B29D 7/01

(52) U.S. Cl. CPC A61K 9/7084; A61K 31/485; A61K 31/465; B29K 2683/00; A61K 47/32; B29D 7/01; A61K 31/27

(57) ABSTRACT

The present invention relates to transdermal therapeutic systems (TTS) for the transdermal administration of an active agent comprising an active agent-containing layer structure, said active agent-containing layer structure comprising A) a backing layer, B) an active agent-containing layer, wherein the active agent-containing layer comprises a therapeutically effective amount of the active agent and at least one silicone acrylic hybrid polymer, and C) a skin contact layer, wherein the skin contact layer comprises at least one silicone acrylic hybrid polymer.

### TECHNICAL FIELD OF THE INVENTION

The present invention relates to a transdermal therapeutic system (TTS) for the transdermal administration of an active agent, processes of manufacture and uses thereof, and methods of treatment therewith.

### BACKGROUND OF THE INVENTION

Transdermal therapeutic systems (TTS) for the transdermal administration of active agents have several advantages over other application systems. In comparison to oral dosage forms, for example, fewer side effects are observed. Furthermore, due to the simple mode of application, more convenience is accomplished for the patient. In particular, longer administration periods on the skin of human patients are beneficial for the compliance. On the other hand it is technically challenging to provide TTS with constant permeation rates for time periods as needed and with the desired physical properties (e.g., tackiness and wear properties). For example, a high loading of the active agent may be required in order to be able to provide sufficient permeation rates of the active agent over the entire administration period. However, an increase of the active agent loading seems limited, in particular in solvent-based systems. Crystallization of the active agent during storage, for example, may jeopardize the therapeutic success due to insufficient permeation rates of the remaining active agent available for skin absorption. The maintenance of sufficient permeation rates with minimum fluctuation during an extended period of time is thus in particular challenging. Furthermore, a high concentration of active agent in the TTS matrix may negatively affect the desired physical properties of the TTS and may cause skin irritation.

The use of an additional skin-contact layer attached to the active agent-containing layer can reduce adverse effects to the skin but may also negatively affect the release profile of the active agent. The delivery of the active agent may then be, for example, too slow at the beginning of the dosing period and/or insufficient to provide a therapeutically effect. Moreover, WO2013/088254 shows, for example, that an additional skin contact layer attached to a buprenorphine-containing matrix layer based on polysiloxanes does not inevitable result in a more constant release of active agent, i.e. a reduced fluctuation of the permeation rate over the administration period.

To reduce the variability of the permeation rate provided by a TTS, it is also required that the TTS, and in particular the area of release of the TTS, remains in contact with the skin during the administration period. A discontinuous contact of the TTS, and in particular of the active agent-containing layer structure, with the skin may result in a reduced and uncontrolled release of the active agent over the administration period. It is thus desirable to not only provide a TTS with a sufficient release performance but, in addition, to provide a TTS with a sufficient tack of the active agent-containing layer structure. The provision of the combination of the described beneficial characteristics of a TTS is particularly challenging in view of the basic requirements for a TTS for being chemical and physical stable and feasible to manufacture on a commercial scale.

There continues to exist a need in the art for an improved TTS that overcomes the above-mentioned disadvantages and provides a continuous administration of the active agent over an extended period of time with a constant delivery of active agent which is sufficient for achieving a therapeutical effect.

### OBJECTS AND SUMMARY OF THE INVENTION

It is an object of the present invention to provide a TTS for the transdermal administration of an active agent that provides a permeation rate which is sufficient for achieving a therapeutically effective dose without negatively affecting the desired physical properties of the TTS (e.g., tackiness and wear properties).

It is a further object of the present invention to provide a TTS for the transdermal administration of an active agent that provides a constant release of the active agent over an extended period of time without negatively affecting the desired physical properties of the TTS (e.g., tackiness and wear properties).

It is a further object of the present invention to provide a TTS for the transdermal administration of an active agent with a high active-agent utilization, i.e. a TTS, which does not require a high excess amount of active agent in order to provide a sufficient release performance during an administration period.

It is a further object of the present invention to provide a TTS for the transdermal administration of an active agent, wherein the adhesive properties of the TTS can be adjusted without negatively affecting the release performance and the active-agent utilization of the TTS.

It is a further object of the present invention to provide a TTS for the transdermal administration of an active agent with good adhesive properties (e.g. a sufficient tack), a good release performance (e.g. a sufficient permeation rate) and a high active agent utilization.

It is a further object of the present invention to provide a TTS for the transdermal administration of an active agent, which provides a sufficiently reproducible release of the active agent over the desired administration period.

It is an object of certain embodiments of the present invention to provide a TTS for the transdermal administration of active agent that is easy to manufacture.

These objects and others are accomplished by the present invention which according to one aspect relates to a transdermal therapeutic system for the transdermal administration of an active agent comprising an active agent-containing layer structure,

the active agent-containing layer structure comprising:

A) a backing layer;

B) an active agent-containing layer,
wherein the active agent-containing layer comprises
a) a therapeutically effective amount of the active agent, and
b) at least one silicone acrylic hybrid polymer;
and

C) a skin contact layer,
wherein the skin contact layer comprises at least one silicone acrylic hybrid polymer.

It has been found that the TTS according to the present invention, which comprises a silicone acrylic hybrid polymer in the active agent-containing layer and in the skin contact layer of an agent-containing layer structure, provides advantageous properties in terms of the constant and continuous active agent delivery, the release performance, the active agent utilization, and the adhesive properties. In particular, the TTS according to the present invention provides the advantageous properties over an extended period of time.

According to certain aspects, the TTS according to the invention is for use in a method of treating pain wherein the transdermal therapeutic system is applied to the skin of a patient preferably for about 24 hours, for more than 3 days, for about 3.5 days, for about 4 days, for about 5 days, or for about 6 days, more preferably for about 7 days. According to certain aspects, the invention relates to a method of treating pain by applying a transdermal therapeutic system in accordance with the invention to the skin of a patient, in particular for about 24 hours, for more than 3 days, for about 3.5 days, for about 4 days, for about 5 days, or for about 6 days, more preferably for about 7 days. In this connection, the active agent is preferably buprenorphine.

According to certain aspects, the TTS according to the invention is for use in a method of treating nicotine addiction, in a method of smoking cessation treatment, in a method of treating Parkinson's disease or in a method of treating Alzheimer's disease wherein the transdermal therapeutic system is applied to the skin of a patient preferably for more than 8 hours, or for about 12 hours, more preferably for at least 24 hours. According to certain aspects, the invention relates to a method of treating nicotine addiction, a method of smoking cessation treatment, a method of treating Parkinson's disease or a method of treating Alzheimer's disease by applying to the skin of a patient a transdermal therapeutic system in accordance with the invention to the skin of a patient, in particular for more than 8 hours, or for about 12 hours, more preferably for at least 24 hours. In this connection, the active agent is preferably nicotine.

According to certain aspects, the TTS according to the invention is for use in a method of preventing, treating, or delaying of progression of Alzheimer's disease, dementia associated with Parkinson's disease, and/or symptoms of traumatic brain injury, or mild to moderate dementia caused by Alzheimer's or Parkinson's disease, wherein the transdermal therapeutic system is applied to the skin of a patient, preferably for at least 24 hours, more preferably for about 24 hours. According to certain aspects, the invention relates to a method of preventing, treating, or delaying of progression of Alzheimer's disease, dementia associated with Parkinson's disease, and/or symptoms of traumatic brain injury, or mild to moderate dementia caused by Alzheimer's or Parkinson's disease by applying to the skin of a patient a transdermal therapeutic system in accordance with the invention, preferably for at least 24 hours, more preferably for about 24 hours. In this connection, the active agent is preferably rivastigmine.

According to yet another aspect, the invention relates to a method of manufacture of a transdermal therapeutic system in accordance with the invention, comprising the steps of:
1) providing an active agent-containing coating composition comprising
   a) the active agent,
   b) optionally a solvent, and
2) coating the active agent-containing coating composition onto a film in an amount to provide the desired area weight,
3) drying the coated active agent-containing coating composition to provide the active agent-containing layer,
4) providing an additional skin contact layer by coating and drying an additional coating composition according to steps 2 and 3, wherein the film is a release liner,
5) laminating the adhesive side of the skin contact layer onto the adhesive side of the active agent-containing layer to provide an active agent-containing layer structure with the desired area of release,
6) punching the individual systems from the active agent-containing layer structure,
7) optionally adhering to the individual systems an active agent-free self-adhesive layer structure comprising also a backing layer and an active agent-free pressure-sensitive adhesive layer and which is larger than the individual systems of active agent-containing self-adhesive layer structure,
   wherein at least one silicone acrylic hybrid polymer composition is added to the active agent-containing coating composition in step 1 and at least one silicone acrylic hybrid polymer composition is added to the additional coating composition in step 4.

### Definitions

Within the meaning of this invention, the term “transdermal therapeutic system” (TTS) refers to a system by which the active agent is administered via transdermal delivery, for example, to the local area to be treated or the systemic circulation and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “active agent-containing layer structure” refers to the layer structure containing a therapeutically effective amount of the active agent and comprises a backing layer, at least one active agent-containing layer and a skin contact layer. Preferably, the active agent-containing layer structure is an active agent-containing self-adhesive layer structure.

Within the meaning of this invention, the term “therapeutically effective amount” refers to a quantity of active agent in the TTS which is, if administered by the TTS to a patient, sufficient to provide a treatment, such as exemplarily the treatment of pain. A TTS usually contains more active in the system than is in fact provided to the skin and the systemic circulation. This excess amount of active agent is usually necessary to provide enough driving force for the delivery from the TTS through the skin and, if desired, into the systemic circulation.

Within the meaning of this invention, the terms “active”, “active agent”, and the like (such as exemplarily the terms “rivastigmine”, “nicotine” and “buprenorphine”) refer to the active agent in any pharmaceutically acceptable chemical and morphological form and physical state. These forms include without limitation the active agent in its free base/free acid form, protonated or partially protonated form of the active agent, their salts, and in particular acid/base addition salts formed by addition of an inorganic or organic acid/base such as hydrochlorides, maleates, solvates, hydrates, clathrates, complexes and so on, as well as active agents in the form of particles which may be micronized, crystalline and/or amorphous, and any mixtures of the aforementioned forms. The active agent, where contained in a medium such as a solvent, may be dissolved or dispersed or in part dissolved and in part dispersed.

When the active agent is mentioned to be used in a particular form in the manufacture of the TTS, this does not exclude interactions between this form of the active agent and other ingredients of the active agent-containing layer structure, e.g. salt formation or complexation, in the final TTS. This means that, even if the active agent is included in its free base/acid form, it may be present in the final TTS in protonated or partially protonated/or deprotonated or partially deprotonated form or in the form of an acid addition salt, or, if it is included in the form of a salt, parts of it may be present as free base in the final TTS. Unless otherwise indicated, in particular the amount of the active agent in the layer structure relates to the amount of active agent included in the TTS during manufacture of the TTS. For example, the amount of buprenorphine is calculated based on buprenorphine in the form of the free base. E.g., when a) 0.1 mmol (equal to equal to 46.76 mg) buprenorphine base or b) 0.1 mmol (equal to 50.41 mg) buprenorphine hydrochloride is included in the TTS during manufacture, the amount of buprenorphine in the layer structure is, within the meaning of the invention, in both cases 46.76 mg, i.e. 0.1 mmol.

The active agent starting material included in the TTS during manufacture of the TTS may be in the form of particles and/or dissolved. The active agent may e.g. be present in the active agent-containing layer structure in the form of particles and/or dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “deposit” as used in reference to “dispersed deposits” refers to distinguishable, e.g., visually distinguishable, areas within the biphasic matrix layer. Such deposits are e.g., droplets and spheres. Within the meaning of this invention, the term droplets is preferably used for deposits in a biphasic coating composition and the term spheres is preferably used for deposits in a biphasic matrix layer. The deposits may be identified by use of a microscope. The sizes of the deposits can be determined by an optical microscopic measurement (for example by Leica MZ16 including a camera, for example Leica DSC320) by taking pictures of the biphasic matrix layer at different positions at an enhancement factor between 10 and 400 times, depending on the required limit of detection. By using imaging analysis software, the sizes of the deposits can be determined.

Within the meaning of this invention, the size of the deposits refers to the diameter of the deposits as measured using a microscopic picture of the biphasic matrix layer.

There are two main types of TTS for active agent delivery, i.e. matrix-type TTS and reservoir-type TTS. The release of the active agent in a matrix-type TTS is mainly controlled by the matrix including the active agent itself. In contrast thereto, a reservoir-type TTS typically needs a rate-controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture and easy and convenient to use by patients.

Within the meaning of this invention, “matrix-type TTS” refers to a system or structure wherein the active is homogeneously dissolved and/or dispersed within a polymeric carrier, i.e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the active agent-containing layer may in one embodiment of the invention be an active agent-containing matrix layer, wherein the active agent is homogeneously distributed within a polymer matrix. In certain embodiments, the active agent-containing matrix layer may comprise two active agent-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the active agent-containing matrix layer may also be referred to as active agent-containing pressure sensitive adhesive layer or active agent-containing pressure sensitive adhesive matrix layer. A TTS comprising the active agent dissolved and/or dispersed within a polymeric gel, e.g. a hydrogel, is also considered to be of matrix-type in accordance with present invention.

TTS with a liquid active agent-containing reservoir are referred to by the term “reservoir-type TTS”. In such a system, the release of the active agent is preferably controlled by a rate-controlling membrane. In particular, the reservoir is sealed between the backing layer and the rate-controlling membrane. Accordingly, the active agent-containing layer may in one embodiment be an active agent-containing reservoir layer, which preferably comprises a liquid reservoir comprising the active agent. Furthermore, the reservoir-type TTS additionally comprises a skin contact layer, wherein the reservoir layer and the skin contact layer may be separated by the rate-controlling membrane. In the reservoir layer, the active agent is preferably dissolved in a solvent such as ethanol or water or in silicone oil. The skin contact layer typically has adhesive properties.

Reservoir-type TTS are not to be understood as being of matrix-type within the meaning of the invention. However, microreservoir TTS (biphasic systems having deposits (e.g. spheres, droplets) of an inner active-containing phase dispersed in an outer polymer phase), considered in the art to be a mixed from of a matrix-type TTS and a reservoir-type TTS that differ from a homogeneous single phase matrix-type TTS and a reservoir-type TTS in the concept of drug transport and drug delivery, are considered to be of matrix-type within the meaning of the invention. The sizes of microreservoir droplets can be determined by an optical microscopic measurement as described above. Without wishing to be bound to any theory it is believed that the size and size distribution of the deposits influences the active agent delivery from the TTS. Large deposits release the active agent too fast and provide for an undesired high active agent delivery at the beginning of the dosing period and a failure of the system for longer dosing periods.

Within the meaning of this invention, the term “active agent-containing layer” refers to a layer containing the active agent and providing the area of release. The term covers active agent-containing matrix layers and active agent-containing reservoir layers. If the active agent-containing layer is an active agent-containing matrix layer, said layer is present in a matrix-type TTS. Additionally, an adhesive overlay may be provided. The additional skin contact layer is typically manufactured such that it is active agent-free. However, due to the concentration gradient, the active agent will migrate from the matrix layer to the additional skin contact layer over time, until equilibrium is reached. The additional skin contact layer may be present on the active agent-containing matrix layer or separated from the active agent-containing matrix layer by a membrane, preferably a rate controlling membrane. If the active agent-containing layer is an active agent-containing reservoir layer, said layer is present in a reservoir-type TTS, and the layer comprises the active agent in a liquid reservoir. The additional skin contact layer is present, in order to provide adhesive properties. Preferably, a rate-controlling membrane separates the reservoir layer from the additional skin contact layer. The additional skin contact layer can be manufactured such that it is active agent-free or active agent-containing. If the additional skin contact layer is free of active agent the active agent will migrate, due to the concentration gradient, from the reservoir layer to the skin contact layer over time, until equilibrium is reached. Additionally an adhesive overlay may be provided.

Within the meaning of this invention, the term “skin contact layer” refers to the layer included in the active agent-containing layer structure to be in direct contact with the skin of the patient during administration. The other layers (e.g. the active agent-containing layer) of the active agent-containing layer structure according to the invention do not contact the skin and do not necessarily have self-adhesive properties. As outlined above, an additional skin contact layer attached to the active agent-containing layer may over time absorb parts of the active agent. The sizes of the skin contact layer and the active agent-containing layer are usually coextensive and correspond to the area of release. However, the area of the additional skin contact layer may also be greater than the area of the active agent-containing layer. In such a case, the area of release still refers to the area of the active agent-containing layer. The skin contact layer of the TTS in accordance with the present invention comprises at least one silicone acrylic hybrid polymer. Preferably, at least one silicone acrylic hybrid polymer is a silicone acrylic hybrid pressure-sensitive adhesive.

As used herein, the active agent-containing layer and the skin contact layer are preferably matrix layers, and it is referred to the final solidified layers. Preferably, a matrix layer is obtained after coating and drying the solvent-containing coating composition as described herein. Alternatively a matrix layer is obtained after melt-coating and cooling. The matrix layer may also be manufactured by laminating two or more such solidified layers (e.g. dried or cooled layers) of the same composition to provide the desired area weight. The matrix layer may be self-adhesive (in the form of a pressure sensitive adhesive matrix layer). Preferably, the matrix layer is a pressure sensitive adhesive matrix layer.

As used herein, an active agent-containing matrix layer is a layer containing the active agent dissolved or dispersed in at least one silicone acrylic hybrid polymer, or containing the active agent dissolved in a solvent to form an active agent-solvent mixture that is dispersed in the form of deposits (in particular droplets) in at least one silicone acrylic hybrid polymer. Preferably, the at least one silicone acrylic hybrid polymer is a silicone acrylic hybrid pressure-sensitive adhesive. Within the meaning of this invention, the terms “pressure-sensitive adhesive layer” and “pressure-sensitive adhesive matrix layer” refer to a pressure-sensitive adhesive layer obtained from a solvent-containing adhesive coating composition after coating on a film and evaporating the solvents.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i.e. provides adhesion to the skin so that typically no further aid for fixation on the skin is needed. A “self-adhesive” layer structure includes a pressure sensitive adhesive layer for skin contact which may be provided in the form of a pressure sensitive adhesive matrix layer. An adhesive overlay may still be employed to advance adhesion.

Within the meaning of this invention, the term “silicone acrylic hybrid polymer” refers to a polymerization product including repeating units of a silicone sub-species and an acrylate-sub species. The silicone acrylic hybrid polymer thus comprises a silicone phase and an acrylic phase. The term “silicone acrylic hybrid” is intended to denote more than a simple blend of a silicone-based sub-species and an acrylate-based sub-species. Instead, the term denotes a polymerized hybrid species that includes silicone-based sub-species and acrylate-based sub-species that have been polymerized together. The silicone acrylic hybrid polymer may also be referred to as a “silicone acrylate hybrid polymer” as the terms acrylate and acrylic are generally used interchangeably in the context of the hybrid polymers used in the present invention.

Within the meaning of this invention, the term “silicone acrylic hybrid pressure-sensitive adhesive” refers to a silicone acrylic hybrid polymer in the form of a pressure-sensitive adhesive. Silicone acrylic hybrid pressure-sensitive adhesives are described, for example, in EP 2 599 847 and WO 2016/130408. Examples of silicone acrylic hybrid pressure-sensitive adhesives include the PSA series 7-6100 and 7-6300 manufactured and supplied in n-heptane or ethyl acetate by Dow Corning (7-610X and 7-630X; X=1 n-heptane-based/X=2 ethyl acetate-based). It was found that, depending on the solvent in which the silicone acrylic hybrid PSA is supplied, the arrangement of the silicone phase and the acrylic phase providing a silicone or acrylic continuous external phase and a corresponding discontinuous internal phase is different. If the silicone acrylic hybrid PSA is supplied in n-heptane, the composition contains a continuous, silicone external phase and a discontinuous, acrylic internal phase. If the silicone acrylic hybrid PSA composition is supplied in ethyl acetate, the composition contains a continuous, acrylic external phase and a discontinuous, silicone internal phase.

Within the meaning of this invention, the term “non-hybrid polymer” is used synonymously for a polymer which does not include a hybrid species. Preferably, the non-hybrid polymer is a pressure-sensitive adhesive (e.g. a silicone- or acrylate-based pressure-sensitive adhesives).

Within the meaning of this invention, the term “silicon-containing pressure-sensitive adhesive composition comprising acrylate or methacrylate functionality” comprises the condensation reaction product of a silicone resin, a silicone polymer, and a silicon-containing capping agent which provides said acrylate or methacrylate functionality. It is to be understood that the silicon-containing pressure-sensitive adhesive composition comprising acrylate or methacrylate functionality can include only acrylate functionality, only methacrylate functionality, or both acrylate functionality and methacrylate functionality.

Within the meaning of this invention, the term “area weight” refers to the dry weight of a specific layer, e.g. of the matrix layer, provided in g/m². The area weight values are subject to a tolerance of ±10%, preferably ±7.5%, due to manufacturing variability.

If not indicated otherwise “%” refers to weight-%.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “cross-linking agent” refers to a substance which is able to cross-link functional groups contained within the polymer.

Within the meaning of this invention, the term “adhesive overlay” refers to a self-adhesive layer structure that is free of active agent and larger in area than the active agent-containing structure and provides additional area adhering to the skin, but no area of release of the active agent. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the active agent-containing layer or forms the backing of the adhesive overlay. At least one backing layer in the TTS and usually the backing layer of the active agent-containing layer is substantially impermeable to the active agent contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates. Also suitable are woven or non-woven backing materials.

The TTS according to the present invention can be characterized by certain parameters as measured in an in vitro skin permeation test or an in vitro permeation test using a EVA-membrane.

Where not otherwise indicated, the skin permeation test is performed with dermatomed split-thickness human skin with a thickness of 800 μm and an intact epidermis, and with phosphate buffer pH 5.5 as receptor medium (32° C. with 0.1% saline azide). Where not otherwise indicated, the permeation test using a EVA-membrane is performed with a EVA-membrane (9% vinyl acetate; Scotchpak Cotran 9702 from 3M) having a thickness of 50 μm and a phosphate buffer pH 5.5 with 0.1% sodium azide at a temperature of 32±1° C. The amount of active permeated into the receptor medium is determined in regular intervals using a validated HPLC method with a UV photometric detector by taking a sample volume. The receptor medium is completely or in part replaced by fresh medium when taking the sample volume, and the measured amount of active permeated relates to the amount permeated between the two last sampling points and not the total amount permeated so far.

Within the meaning of this invention, the parameter “permeated amount” is provided in μg/cm² and relates to the amount of active permeated in a sample interval at certain elapsed time. E.g., in a permeation test as described above, wherein the amount of active permeated into the receptor medium has been e.g. measured at hours 0, 8, 24, 32, 48 and 72, the “permeated amount” of active can be given e.g. for the sample interval from hour 32 to hour 48 and corresponds to the measurement at hour 48, wherein the receptor medium has been exchanged completely at hour 32.

The permeated amount can also be given as a “cumulative permeated amount”, corresponding to the cumulated amount of active permeated at a certain point in time. E.g., in a permeation test as described above, wherein the amount of active permeated into the receptor medium has been e.g. measured at hours 0, 8, 24, 32, 48 and 72, the “cumulative permeated amount” of active at hour 48 corresponds to the sum of the permeated amounts from hour 0 to hour 8, hour 8 to hour 24, hour 24 to hour 32, and hour 32 to hour 48.

Within the meaning of this invention, the parameter “permeation rate” for a certain sample interval at certain elapsed time is provided in μg/cm²-hr and is calculated from the permeated amount in said sample interval as measured by a permeation test as described above in μg/cm², divided by the hours of said sample interval. E.g. the permeation rate in a permeation test as described above, wherein the amount of active permeated into the receptor medium has been e.g. measured at hours 0, 8, 24, 32, 48 and 72, the “permeation rate” at hour 48 is calculated as the permeated amount in the sample interval from hour 32 to hour 48 divided by 16 hours.

A “cumulative permeation rate” can be calculated from the respective cumulative permeated amount by dividing the cumulative permeated amount by the elapsed time. E.g. in a permeation test as described above, wherein the amount of active permeated into the receptor medium has been e.g. measured at hours 0, 8, 24, 32, 48 and 72, the “cumulative permeation rate” at hour 48 is calculated as the cumulative permeated amount at hour 48 (see above) divided by 48 hours.

Within the meaning of this invention, the term “release performance” refers to the parameters which express the release of the active agent per cm², such as the “permeated amount”, the “cumulative permeated amount”, the “permeation rate” and the “cumulative permeation rate”.

Within the meaning of this invention, the term “active agent utilization” refers to the cumulative permeated amount after a certain elapsed time, e.g. after 24 hours, divided by the initial loading of the active agent.

Within the meaning of this invention, the above parameters “permeated amount” and “permeation rate” (as well as “cumulative permeated amount” and “cumulative permeation rate”) refer to mean values calculated from at least 3 permeation test experiments. Where not otherwise indicated, the standard deviation (SD) of these mean values refer to a corrected sample standard deviation, calculated using the formula:

$$SD = \sqrt{\frac{1}{n - 1} + {\sum }_{i = 1}^{n} {( {x}_{i} - \overline{x} )}^{2}}$$

wherein n is the sample size, {x₁, x₂, . . . xₙ} are the observed values and x̅ is the mean value of the observed values.

Within the meaning of this invention, the term “extended period of time” relates to a period of at least or about 8 hours, at least or about 12 hours, at least or about 24 hours (1 day), at least or about 32 hours, at least or about 48 hours, at least or about 72 hours (3 days), at least or about 84 hours (3.5 days), at least or about 96 hours (4 days), at least or about 120 hours (5 days), at least or about 144 hours (6 days), or at least or about 168 hours (7 days).

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention, the term “coating composition” refers to a composition comprising all components of the matrix layer in a solvent, which may be coated onto the backing layer or release liner to form the matrix layer upon drying.

Within the meaning of this invention, the term “pressure sensitive adhesive composition” refers to a pressure sensitive adhesive at least in mixture with a solvent (e.g. n-heptane or ethyl acetate).

Within the meaning of this invention, the term “dissolve” refers to the process of obtaining a solution, which is clear and does not contain any particles, as visible to the naked eye.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof.

### BRIEF DESCRIPTION OF THE DRAWINGS

FIG. 1a depicts the permeation rates of Comparative Examples 1 and 2 over a time interval of 168 hours.

FIG. 1b depicts the cumulative permeated amount of Comparative Examples 1 and 2 over a time interval of 168 hours.

FIG. 2a depicts the permeation rate of Example 1a, Example 1b and Comparative Example 1 over a time interval of 168 hours.

FIG. 2b depicts the cumulative permeated amount of Example 1a, Example 1b and Comparative Example 1 over a time interval of 168 hours.

FIG. 2c depicts the results of the measurement of the tack, the cumulative permeated amount of active agent and the active agent utilization of Example 1a, Example 1b and Comparative Example 2 in comparison to Comparative Example 1.

FIG. 3a depicts the permeation rates of Example 2a, Example 2b and Comparative Examples 3 and 4 over a time interval of 32 hours.

FIG. 3b depicts the cumulative permeated amount of Example 2a, Example 2b and Comparative Examples 3 and 4 over a time interval of 32 hours.

FIG. 4a depicts the permeation rates of Example 3a, Example 3b, Example 3c, Example 3d and Comparative Example 5 over a time interval of 24 hours.

FIG. 4b depicts the cumulative permeated amount of Example 3a, Example 3b, Example 3c, Example 3d and Comparative Example 5 over a time interval of 24 hours.

### DETAILED DESCRIPTION

### TTS Structure

The present invention relates to a transdermal therapeutic system for the transdermal administration of an active agent comprising an active agent-containing layer structure.

The active agent-containing layer structure according to the invention comprises A) a backing layer, B) an active agent-containing layer and C) a skin contact layer. The active agent-containing layer structure is preferably an active agent-containing self-adhesive layer structure.

The active agent-containing layer according to the invention comprises a therapeutically effective amount of the active agent and at least one silicone acrylic hybrid polymer. The skin contact layer according to the invention also comprises at least one silicone acrylic hybrid polymer.

Thus, in a first aspect, the present invention relates to a transdermal therapeutic system for the transdermal administration of an active agent comprising an active agent-containing layer structure,

the active agent-containing layer structure comprising:

A) a backing layer;

B) an active agent-containing layer,
wherein the active agent-containing layer comprises
a) a therapeutically effective amount of the active agent, and
b) at least one silicone acrylic hybrid polymer;
and

C) a skin contact layer,
wherein the skin contact layer comprises at least one silicone acrylic hybrid polymer.

In certain embodiments of the invention, the at least one silicone acrylic hybrid polymer contained in the active agent-containing layer is the same as the at least one silicone acrylic hybrid polymer contained in the skin contact layer. In certain other embodiments of the invention, the at least one silicone acrylic hybrid polymer contained in the active agent-containing layer is different compared to the at least one silicone acrylic hybrid polymer contained in the skin contact layer.

In a preferred embodiment of the invention, the silicone acrylic hybrid polymer is a silicone acrylic hybrid pressure sensitive adhesive. Further details regarding the silicone acrylic hybrid polymer according to the invention are provided further below.

The backing layer is in particular substantially active agent-impermeable.

The active agent-containing layer may be directly attached to the backing layer, so that no further layer between the backing layer and the active agent-containing layer is present.

In one embodiment of the present invention, at least one additional layer may be between the active agent-containing layer and the skin contact layer. It is however preferred that the skin contact layer is attached to the active agent-containing layer.

The TTS according to the present invention may be a matrix-type TTS or a reservoir-type TTS, and preferably is a matrix-type TTS.

The active agent-containing layer structure according to the invention is normally located on a detachable protective layer (release liner), from which it is removed immediately before application to the surface of the patient's skin. Thus, the TTS may further comprise a release liner. A TTS protected this way is usually stored in a blister pack or a seam-sealed pouch.

The packaging may be child resistant and/or senior friendly.

According to certain embodiments of the invention, the TTS may further comprise an adhesive overlay. This adhesive overlay is in particular larger in area than the active agent-containing structure and is attached thereto for enhancing the adhesive properties of the overall transdermal therapeutic system. Said adhesive overlay comprises a backing layer and an adhesive layer. The adhesive overlay provides additional area adhering to the skin but does not add to the area of release of the active agent. The adhesive overlay comprises a self-adhesive polymer or a self-adhesive polymer mixture selected from the group consisting of silicone acrylic hybrid polymers, acrylic polymers, polysiloxanes, polyisobutylenes, styrene-isoprene-styrene copolymers, and mixtures thereof, which may be identical to or different from any polymer or polymer mixture included in the active agent-containing layer structure. In one embodiment, the TTS is free of an adhesive overlay on top of the active agent-containing layer structure.

In certain embodiments of the invention, the active agent-containing layer structure provides a tack of from 0.6 N to 8.0 N, preferably from more than 0.8 N to 8.0 N, or from 0.9 N to 8.0 N, or from more than 0.9 N to 8.0 N, or from 1.2 N to 6.0 N, or from more than 1.2 N to 6.0 N, preferably determined in accordance with the Standard Test Method for Pressure-Sensitive Tack of Adhesives Using an Inverted Probe Machine (ASTM D 2979-01; Reapproved 2009), wherein the transdermal therapeutic system samples were equilibrated 24 hours under controlled conditions at approx. room temperature (23±2° C.) and approx. 50% rh (relative humidity) prior to testing.

In certain embodiments of the invention, the active agent-containing layer structure provides an adhesion force of from about 2 N/25 mm to about 16 N/25 mm, preferably of from about 3.5 N/25 mm to about 15 N/25 mm, more preferably of from about 4 N/25 mm to about 15 N/25 mm, preferably determined using a tensile strength testing machine with an aluminium testing plate and a pull angle of 90°, wherein the transdermal therapeutic system samples were equilibrated 24 hours under controlled conditions at approx. room temperature (23±2° C.) and approx. 50% rh (relative humidity) prior to testing and are cut into pieces with a fixed width of 25 mm.

In one particular embodiment, the present invention relates to a transdermal therapeutic system for the transdermal administration of active agent comprising an active agent-containing layer structure,
the active agent-containing layer structure comprising:
A) a backing layer;
B) an active agent-containing matrix layer;
   wherein the active agent-containing matrix layer comprises
   a) the active agent in an amount of from 5 to 35% by weight based on the active agent-containing matrix layer, and
   b) a silicone acrylic hybrid polymer in an amount of from about 30% to about 95% by weight based on the active agent-containing matrix layer;
   and
C) a skin contact layer on the active agent-containing matrix layer comprising at least one silicone acrylic hybrid polymer in an amount of from about 50% to about 100% by weight based on the skin contact layer, wherein the silicone acrylic hybrid polymer is a silicone acrylic hybrid pressure-sensitive adhesive having a weight ratio of silicone to acrylate of from 40:60 to 60:40, preferably wherein the ethylenically unsaturated monomers forming the acrylate comprise 2-ethylhexyl acrylate and methyl acrylate in a ratio of from 65:35 to 55:45.

In one particular embodiment, the present invention relates to a transdermal therapeutic system for the transdermal administration of rivastigmine comprising a rivastigmine-containing layer structure,
the rivastigmine-containing layer structure comprising:
A) a backing layer;
B) a rivastigmine-containing matrix layer;
   wherein the rivastigmine-containing matrix layer comprises
   a) a therapeutically effective amount of rivastigmine and
   b) at least one silicone acrylic hybrid polymer;
   and
C) a skin contact layer on the rivastigmine-containing matrix layer comprising at least one silicone acrylic hybrid pressure-sensitive adhesive.

In certain embodiments of the invention, the transdermal therapeutic system further comprises at least one non-hybrid polymer, preferably at least one non-hybrid polymer based on polysiloxanes, polyisobutylenes, styrene-isoprene-styrene block copolymers, or acrylates. The at least one non-hybrid polymer may be contained in the active agent-containing layer, in the skin contact layer, or in both the active agent-containing layer and the skin contact layer. In a particular preferred embodiment, the at least one non-hybrid polymer is a non-hybrid pressure-sensitive adhesive, preferably based on polysiloxanes, polyisobutylenes, styrene-isoprene-styrene block copolymers, or acrylates, more preferably based on polysiloxanes or acrylates. Further details regarding the non-hybrid polymers according to the invention are provided further below.

### Active Agent-Containing Layer

As outlined in more detail above, the agent-containing layer structure of the TTS according to the present invention comprises a backing layer, an active agent-containing layer, and a skin contact layer. The active agent-containing layer comprises a therapeutically effective amount of the active agent and at least one silicone acrylic hybrid polymer.

The active agent-containing layer may be an active agent-containing matrix layer or an active agent-containing reservoir layer. It is preferred that the active agent-containing layer is an active agent-containing matrix layer.

In one embodiment, the active agent-containing layer is a self-adhesive active agent-containing layer, more preferably a self-adhesive active agent-containing matrix layer.

In a certain embodiment, the active agent-containing layer is obtainable by coating and drying an active agent-containing coating composition that comprises a therapeutically effective amount of the active agent and the at least one silicone acrylic hybrid polymer.

In a certain embodiment, the silicone acrylic hybrid polymer in the active agent-containing layer contains a continuous, silicone external phase and a discontinuous, acrylic internal phase. In a certain other embodiment, the silicone acrylic hybrid polymer in the active agent-containing layer contains a continuous, acrylic external phase and a discontinuous, silicone internal phase.

In certain embodiments, the active agent-containing layer contains the silicone acrylic hybrid polymer in an amount of from about 20% to about 98%, from about 30% to about 95%, or from about 50% to about 95% by weight based on the active agent-containing layer.

In a certain embodiment, the active agent-containing layer has a continuous, silicone external phase and a discontinuous, acrylic internal phase. In a certain other embodiment, the active agent-containing layer has a continuous, acrylic external phase and a discontinuous, silicone internal phase.

In a certain preferred embodiment, the active agent-containing layer is an active agent-containing matrix layer and contains the silicone acrylic hybrid polymer in an amount of from about 30% to about 95% by weight based on the active agent-containing matrix layer, preferably wherein the silicone acrylic hybrid polymer is a silicone acrylic hybrid pressure-sensitive adhesive having a weight ratio of silicone to acrylate of from 40:60 to 60:40, and wherein the ethylenically unsaturated monomers forming the acrylate comprise 2-ethylhexyl acrylate and methyl acrylate in a ratio of from 40:60 to 70:30.

In one embodiment, the active agent-containing layer is an active agent-containing biphasic matrix layer having an inner phase comprising the therapeutically effective amount of the active agent, and having an outer phase comprising the at least one silicone acrylic hybrid polymer, wherein the inner phase forms dispersed deposits in the outer phase. The content of the inner phase in the biphasic matrix layer is preferably from 5 to 40% by volume based on the volume of the biphasic matrix layer. The dispersed deposits have preferably a maximum sphere size of from about 1 μm to about 80 μm, more preferably of from about 5 μm to about 65 μm.

In a certain embodiment, when the active agent-containing layer is a biphasic matrix layer, the active agent is not dissolved to a large extent within the polymer of the outer phase of the biphasic matrix layer but within the inner phase, which forms the microreservoirs incorporated within the polymer of the phase.

In certain embodiments, the active agent is contained in an amount of from 2% to 40%, preferably from 3% to 40%, more preferably from 5% to 35% by weight based on the active agent-containing layer.

According to a certain embodiments, the active agent-containing layer has an area weight of from 10 to 180 g/m², from 20 to 160 g/m², from 60 to 160 g/m², from 30 to 140 g/m², from 40 to 140 g/m², or from more than 80 to 140 g/m².

In certain embodiments, the active agent-containing layer further comprises a carboxylic acid, preferably in an amount sufficient so that the therapeutically effective amount of the active agent is solubilized therein. In one embodiment, the therapeutically effective amount of active agent is in solution in the carboxylic acid.

In certain embodiments, the carboxylic acid is contained in an amount of from 2% to 20%, preferably from 4% to 15%, more preferably from 5% to 12%, by weight based on the active agent-containing layer.

In certain embodiments, the active agent-containing layer is an active agent-containing biphasic matrix layer having an inner phase comprising the therapeutically effective amount of the active agent and a carboxylic acid, and having an outer phase comprising the at least one silicone acrylic hybrid polymer, wherein the inner phase forms dispersed deposits in the outer phase.

In one embodiment, the active agent and the carboxylic acid are contained in different amounts by weight based on the active agent-containing layer. The active agent and the carboxylic acid may however also be contained in the same amounts by weight based on the active agent-containing layer, such that the carboxylic acid and the active agent are e.g. contained in an amount ratio of about 1:1.

The carboxylic acid may be contained in less amounts by weight than the active agent based on the active agent-containing layer. The active agent may however also be contained in less amounts by weight than the carboxylic acid based on the active agent-containing layer. Preferably, the carboxylic acid and the active agent are contained in the active agent-containing layer in an amount ratio of from 0.3:1 to 5:1.

Suitable carboxylic acid may be selected from the group consisting of C₃ to C₂₄ carboxylic acids. In certain embodiments, the carboxylic acid contained in the active agent-containing layer is selected from the group consisting of oleic acid, linoleic acid, linolenic acid, levulinic acid, and mixtures thereof, in particular the carboxylic acid is levulinic acid. In a particular embodiment, the carboxylic acid is levulinic acid and the levulinic acid and the active agent are contained in the active agent-containing layer in an amount ratio of from 0.3:1 to 5:1.

Since the carboxylic acid, such as e.g., the levulinic acid, can likewise be absorbed through the skin, the amount in the TTS may become less as the time of application elapses, and may lead to a reduction of the solubility of the active agent. As a result, the decrease in the thermodynamic activity of active agent, due to depletion, is then compensated by the reduced drug solubility.

In certain embodiments, the active agent-containing layer, comprising the at least one silicone acrylic hybrid polymer, further comprises at least one non-hybrid polymer such that the active agent-containing layer comprises
a) a therapeutically effective amount of active agent,
b) at least one silicone acrylic hybrid polymer,
c) at least one non-hybrid polymer, and
d) optionally a carboxylic acid.
   In this connection, the at least one silicone acrylic hybrid polymer and at least one non-hybrid polymer may be comprised in the active agent-containing layer in an amount ratio of from 0.1:1 to 5:1, preferably of from 0.5:1 to 2:1. Further details regarding non-hybrid polymers according to the invention are provided further below.

In certain embodiments, the active agent-containing layer is an active agent-containing biphasic matrix layer having an inner phase comprising the therapeutically effective amount of the active agent, and having an outer phase comprising the at least one silicone acrylic hybrid polymer and the at least one non-hybrid polymer, wherein the inner phase forms dispersed deposits in the outer phase.

In certain embodiments, the active agent-containing layer is an active agent-containing biphasic matrix layer having an inner phase comprising the therapeutically effective amount of the active agent and a carboxylic acid, and having an outer phase comprising the at least one silicone acrylic hybrid polymer and the at least one non-hybrid polymer, wherein the inner phase forms dispersed deposits in the outer phase.

In certain embodiments, the active agent-containing layer further comprises an auxiliary polymer. The auxiliary polymer may be contained in an amount of from about 0.5% to about 30% by weight based on the active agent-containing layer, preferably in an amount of from about 2% to about 25% by weight based on the active agent-containing layer. The auxiliary polymer is preferably selected from the group consisting of alkyl methacrylate copolymers, amino alkyl methacrylate copolymers, methacrylic acid copolymers, methacrylic ester copolymers, ammonioalkyl methacrylate copolymers, polyvinylpyrrolidones, vinylpyrrolidone-vinyl acetate copolymers, polyvinyl caprolactam-polyvinyl acetate-polyethylene glycol copolymer, and mixtures thereof. In one embodiment, the auxiliary polymer is a polyvinylpyrrolidone, preferably contained in an amount of from about 0.5% to about 8% by weight based on the active agent-containing layer.

The TTS according to the invention may further comprise one or more anti-oxidants. Suitable anti-oxidants are sodium metabisulfite, ascorbyl palmitate, tocopherol and esters thereof, ascorbic acid, butylhydroxytoluene, butylhydroxyanisole or propyl gallate, preferably sodium metabisulfite, ascorbyl palmitate and tocopherol. The anti-oxidants may be conveniently present in the active agent-containing layer, preferably in an amount of from about 0.001 to about 0.5% of the active agent-containing layer.

The TTS according to the invention may further comprise in addition to the above mentioned ingredients at least one excipient or additive, for example from the group of cross-linking agents, solubilizers, fillers, tackifiers, film-forming agents, plasticizers, stabilizers, softeners, substances for skincare, permeation enhancers, pH regulators, and preservatives. In general, it is preferred according to the invention that no additional excipients or additives are required. Thus, the TTS has a composition of low complexity. In certain embodiments, no further additive (e.g. a tackifier) is present in the TTS.

### Skin Contact Layer

As outlined in more detail above, the agent-containing layer structure of the TTS according to the present invention comprises a backing layer, an active agent-containing layer, and a skin contact layer. The skin contact layer comprises at least one silicone acrylic hybrid polymer, preferably the at least one silicone acrylic hybrid polymer is a silicone acrylic hybrid pressure-sensitive adhesive. Preferably, the skin contact layer is in contact with the active agent-containing layer.

In certain embodiments, the skin contact layer contains the silicone acrylic hybrid polymer in an amount of from about 30% to about 100%, from about 50% to about 100%, or from about 80% to about 100% by weight based on the skin contact layer.

In a certain embodiment, the silicone acrylic hybrid polymer in the skin contact layer contains a continuous, silicone external phase and a discontinuous, acrylic internal phase. In a certain other embodiment, the silicone acrylic hybrid polymer in the active agent-containing layer contains a continuous, acrylic external phase and a discontinuous, silicone internal phase.

In a certain embodiment, the skin contact layer has a continuous, silicone external phase and a discontinuous, acrylic internal phase. In a certain other embodiment, skin contact layer has a continuous, acrylic external phase and a discontinuous, silicone internal phase.

In a certain preferred embodiment, the skin contact layer contains the silicone acrylic hybrid polymer in an amount of from about 50% to about 100% by weight based on the skin contact layer, wherein the silicone acrylic hybrid polymer is a silicon acrylic hybrid pressure-sensitive adhesive having a weight ratio of silicone to acrylate of from 40:60 to 60:40, preferably wherein the ethylenically unsaturated monomers forming the acrylate comprise 2-ethylhexyl acrylate and methyl acrylate in a ratio of from 65:35 to 55:45.

In a certain preferred embodiment, the skin contact layer contains the silicone acrylic hybrid polymer in an amount of from about 80% to about 100% by weight based on the skin contact layer, wherein the silicone acrylic hybrid polymer is a silicon acrylic hybrid pressure-sensitive adhesive having a weight ratio of silicone to acrylate of from 40:60 to 60:40, and wherein the ethylenically unsaturated monomers forming the acrylate comprise 2-ethylhexyl acrylate and methyl acrylate in a ratio of from 65:35 to 55:45, preferably wherein the skin contact layer has a continuous, acrylic external phase and a discontinuous, silicone internal phase.

In a certain preferred embodiment, the skin contact layer contains the silicone acrylic hybrid polymer in an amount of from about 80% to about 100% by weight based on the skin contact layer, wherein the silicone acrylic hybrid polymer is a silicon acrylic hybrid pressure-sensitive adhesive having a weight ratio of silicone to acrylate of from 40:60 to 60:40, and wherein the silicone acrylic hybrid pressure-sensitive adhesive is characterized by a solution viscosity at 25° C. and about 50% solids content in ethyl acetate of from about 1,200 cP to about 1,800 cP, preferably as measured using a Brookfield RVT viscometer equipped with a spindle number 5 at 50 RPM, and/or is characterized by a complex viscosity at 0.1 rad/s at 30° C. of from about 9.0e5 Poise to about 7.0e6 Poise, preferably as measured using a Rheometrics ARES rheometer, wherein the rheometer is equipped with 8 mm plates and the gap zeroed, preferably wherein the skin contact layer has a continuous, acrylic external phase and a discontinuous, silicone internal phase.

In one embodiment, the skin contact layer further comprises at least one non-hybrid polymer.

The skin contact layer may comprise an active agent. In a preferred embodiment, the skin contact layer is free of active agent, that is, is prepared without the addition of an active agent.

In certain embodiments, the skin contact layer, comprising the at least one silicone acrylic hybrid polymer, further comprises at least one non-hybrid polymer. In this connection, the at least one silicone acrylic hybrid polymer and at least one non-hybrid polymer may be comprised in the skin contact layer in an amount ratio of from 0.1:1 to 5:1, preferably of from 0.5:1 to 2:1. Further details regarding non-hybrid polymers according to the invention are provided further below.

The skin contact layer may have an area weight of from 5 to 150 g/m², 20 to 150 g/m², or from 20 to 130 g/m². In certain embodiment, the skin contact layer has an area weight of from 10 to 140 g/m², preferably from 5 to 40 g/m², from 10 to 30 g/m², from 20 to 40 g/m², from 80 to 130 g/m², or from 90 to 120 g/m².

### Active Agent

The TTS according to the invention comprises a therapeutically effective amount of active agent.

The amount of the active agent incorporated into the system varies depending on many factors including, but not limited to, the particular active agent, the desired therapeutic effect, and the time span for which the system is to provide therapy. A therapeutically effective amount may vary from about 1 mg to about 50 mg.

In certain embodiments of the invention, the active agent is contained in an amount of from 2% to 40%, preferably from 3% to 40%, more preferably from 5% to 35% by weight based on the active agent-containing layer.

In certain embodiments of the invention, the active agent is contained in the active agent-containing layer structure in an amount of from 0.3 mg/cm² to 3.0 mg/cm², from 0.5 mg/cm² to 2.5 mg/cm², from 0.6 mg/cm² to 2.2 mg/cm², or from 1.3 mg/cm² to 2.2 mg/cm′. In certain embodiments, the active agent is contained in the active agent-containing layer structure in an amount of from 0.5 mg/cm² to 1.6 mg/cm², more than 0.6 mg/cm² to less than 1.8 mg/cm², 1.2 mg/cm² to less than 1.8 mg/cm², or more than 0.6 mg/cm² to less than 1.2 mg/cm² based on the active agent-containing layer.

The active agent can be any component suitable for transdermal delivery to a patient.

In a certain embodiment according to the present invention, the active agent is an active agent suitable for the systemic treatment, i.e. active agents for administration to the systemic circulation. Suitable active agent include, but are not limited to rivastigmine, nicotine and buprenorphine. In one embodiment, the active agent is nicotine. In another embodiment, the active agent is buprenorphine. In a preferred embodiment, the active agent is rivastigmine. In a certain embodiment of the present invention, the active agent is not buprenorphine or nicotine.

In accordance with the present invention, the active agent may be present in the TTS in any form as defined above. Thus, in certain embodiments, the may be included in the form of the free base (e.g. rivastigmine base, nicotine base, or buprenorphine base). In other certain embodiments, the active may be included in the form of a pharmaceutically acceptable chemical and morphological form and physical state, such as a pharmaceutically acceptable salt thereof.

In certain embodiments of the invention, the active agent is rivastigmine (e.g. rivastigmine base) and is contained in the rivastigmine-containing layer structure in an amount of from 0.3 mg/cm² to 3.0 mg/cm², from 0.5 mg/cm² to 2.5 mg/cm², from 0.6 mg/cm² to 2.2 mg/cm′, or from 1.3 mg/cm² to 2.2 mg/cm′.

In certain embodiments of the invention, the active agent is nicotine (e.g. nicotine base) and is contained in the nicotine-containing layer structure in an amount of from 0.3 mg/cm² to 3.0 mg/cm², from 0.5 mg/cm² to 2.5 mg/cm², from 0.6 mg/cm² to 2.2 mg/cm², or from 1.3 mg/cm² to 2.2 mg/cm′.

In certain embodiments of the invention, the active agent is buprenorphine (e.g. buprenorphine base) and is contained in the buprenorphine-containing layer structure in an amount of from 0.3 mg/cm² to 3.0 mg/cm², from 0.5 mg/cm² to 2.5 mg/cm², from 0.6 mg/cm² to 2.2 mg/cm², or from 1.3 mg/cm² to 2.2 mg/cm′.

According to certain embodiments, the amount of active agent (e.g. rivastigmine base) contained in the transdermal therapeutic system, according to six different dosages, ranges from about 2.5 mg to about 6.5 mg active agent and the size of the active agent-containing layer providing the area of release ranges from about 1 cm² to about 4.5 cm², preferably from about 1 cm² to less than 2.5 cm², or the amount of active agent contained in the transdermal therapeutic system ranges from about 6 mg to about 12 mg active agent and the size of the active agent-containing layer providing the area of release ranges from about 3 cm² to about 7 cm², preferably from about 2.5 cm² to less than 5 cm², or the amount of active agent contained in the transdermal therapeutic system ranges from about 10 mg to about 17 mg active agent and the size of the active agent-containing layer providing the area of release ranges from about 5.5 cm² to about 10 cm², preferably from about 4.5 cm² to less than 7.5 cm², or the amount of active agent contained in the transdermal therapeutic system ranges from about 14 mg to about 22 mg active agent and the size of the active agent-containing layer providing the area of release ranges from about 7 cm² to about 13 cm², preferably from about 6.5 cm² to less than 10 cm², or amount of active agent contained in the transdermal therapeutic system ranges from about 21 mg to about 33 mg active agent and the size of the active agent-containing layer providing the area of release ranges from about 11 cm² to about 19 cm², preferably from about 10.5 cm² to less than 15 cm², or the amount of active agent contained in the transdermal therapeutic system ranges from about 29 mg to about 43 mg active agent and the size of the active agent-containing layer providing the area of release ranges from about 17 cm² to about 23 cm², preferably from about 16 cm² to less than 20 cm², wherein the six different transdermal therapeutic systems have increasing areas of release and amounts of active agent (e.g. rivastigmine base).

According to certain embodiments, the amount of active agent (e.g. rivastigmine base) contained in the transdermal therapeutic system ranges from about 2.5 mg to about 43 mg.

### Silicone Acrylic Hybrid Polymer

The TTS according to the present invention comprises a silicone acrylic hybrid polymer. The silicone acrylic hybrid polymer comprises a polymerized hybrid species that includes silicone-based sub-species and acrylate-based sub-species that have been polymerized together. The silicone acrylic hybrid polymer thus comprises a silicone phase and an acrylic phase. Preferably, the silicone acrylic hybrid polymer is a silicone acrylic hybrid pressure-sensitive adhesive.

The silicone acrylic hybrid pressure-sensitive adhesives are usually supplied and used in solvents like n-heptane and ethyl acetate. The solids content of the pressure-sensitive adhesives is usually between 30% and 80%. The skilled person is aware that the solids content may be modified by adding a suitable amount of solvent.

Preferably, the weight ratio of silicone to acrylate in the silicone acrylic hybrid pressure-sensitive adhesive is from 5:95 to 95:5, or from 20:80 to 80:20, more preferably from 40:60 to 60:40, and most preferably the ratio of silicone to acrylate is about 50:50. Suitable silicone acrylic hybrid pressure-sensitive adhesives having a weight ratio of silicone to acrylate of 50:50 are, for example, the commercially available silicone acrylic hybrid pressure-sensitive adhesives 7-6102, Silicone/Acrylate Ratio 50/50, and 7-6302, Silicone/Acrylate Ratio 50/50, supplied in ethyl acetate by Dow Corning.

The preferred silicone acrylic hybrid pressure-sensitive adhesives in accordance with the invention are characterized by a solution viscosity at 25° C. and about 50% solids content in ethyl acetate of more than about 400 cP, or from about 500 cP to about 3,500 cP, in particular from about 1,000 cP to about 3,000 cP, more preferred from about 1,200 cP to about 1,800, or most preferred of about 1,500 cP or alternatively more preferred from about 2,200 cP to about 2,800 cP, or most preferred of about 2,500 cP, preferably as measured using a Brookfield RVT viscometer equipped with a spindle number 5 at 50 RPM.

These silicone acrylic hybrid pressure-sensitive adhesives may also be characterized by a complex viscosity at 0.1 rad/s at 30° C. of less than about 1.0e9 Poise, or from about 1.0e5 Poise to about 9.0e8 Poise, or more preferred from about 9.0e5 Poise to about 1.0e7 Poise, or most preferred about 4.0e6 Poise, or alternatively more preferred from about 2.0e6 Poise to about 9.0e7 Poise, or most preferred about 1.0e7 Poise, preferably as measured using a Rheometrics ARES rheometer, wherein the rheometer is equipped with 8 mm plates and the gap zeroed.

In one embodiment of the present invention, the transdermal therapeutic system comprises at least two silicone acrylic hybrid polymers selected from at least two of the silicone acrylic hybrid polymer groups:
silicone acrylic hybrid pressure-sensitive adhesives characterized by a solution viscosity at 25° C. and about 50% solids content in ethyl acetate of from about 1,200 cP to about 1,800 cP, preferably as measured using a Brookfield RVT viscometer equipped with a spindle number 5 at 50 RPM, and
silicone acrylic hybrid pressure-sensitive adhesives characterized by a solution viscosity at 25° C. and about 50% solids content in ethyl acetate of from about 2,200 cP to about 2,800 cP, preferably as measured using a Brookfield RVT viscometer equipped with a spindle number 5 at 50 RPM. The at least two silicone acrylic hybrid polymers may be contained in the active agent-containing layer, in the skin contact layer, or in different layers. In one embodiment, the at least two silicone acrylic hybrid polymers are contained in different layers of the TTS.

In another embodiment of the present invention, the transdermal therapeutic system comprises at least two silicone acrylic hybrid polymers selected from at least two of the silicone acrylic hybrid polymer groups:
silicone acrylic hybrid pressure-sensitive adhesives characterized by a complex viscosity at 0.1 rad/s at 30° C. of from about 9.0e5 Poise to about 7.0e6 Poise, preferably as measured using a Rheometrics ARES rheometer, wherein the rheometer is equipped with 8 mm plates and the gap zeroed, and
silicone acrylic hybrid pressure-sensitive adhesives characterized by a complex viscosity at 0.1 rad/s at 30° C. of from about 8.0e6 Poise to about 9.0e7 Poise, preferably as measured using a Rheometrics ARES rheometer, wherein the rheometer is equipped with 8 mm plates and the gap zeroed. The at least two silicone acrylic hybrid polymers may be contained in the active agent-containing layer, in the skin contact layer, or in different layers. In one embodiment, the at least two silicone acrylic hybrid polymers are contained in different layers of the TTS.

To prepare samples for measuring the rheological behavior using a Rheometrics ARES rheometer, between 2 and 3 grams of adhesive solution can be poured onto a SCOTCH-PAK 1022 fluoropolymer release liner and allow to sit for 60 minutes under ambient conditions. To achieve essentially solvent-free films of the adhesive, they can be placed in an oven at 110° C.+/−10° C. for 60 minutes. After removing from the oven and letting equilibrate to room temperature. The films can be removed from the release liner and folded over to form a square. To eliminate air bubbles the films can be compressed using a Carver press. The samples can then be loaded between the plates and are compressed to 1.5+/−0.1 mm at 30° C. The excess adhesive is trimmed and the final gap recorded. A frequency sweep between 0.01 to 100 rad/s can be performed with the following settings: Temperature=30° C.; strain=0.5-1% and data collected at 3 points/decade.

Suitable silicone acrylic hybrid pressure-sensitive adhesives which are commercially available include the PSA series 7-6100 and 7-6300 manufactured and supplied in n-heptane or ethyl acetate by Dow Corning (7-610X and 7-630X; X=1 n-heptane-based/X=2 ethyl acetate-based). For example, the 7-6102 silicone acrylic hybrid PSA having a silicone/acrylate ratio of 50/50 is characterized by a solution viscosity at 25° C. and about 50% solids content in ethyl acetate of 2,500 cP and a complex viscosity at 0.1 rad/s at 30° C. of 1.0e7 Poise. The 7-6302 silicone acrylic hybrid PSA having a silicone/acrylate ratio of 50/50 has a solution viscosity at 25° C. and about 50% solids content in ethyl acetate of 1,500 cP and a complex viscosity at 0.1 rad/s at 30° C. of 4.0e6 Poise.

Depending on the solvent in which the silicone acrylic hybrid pressure-sensitive adhesive is supplied, the arrangement of the silicone phase and the acrylic phase providing a silicone or acrylic continuous external phase and a corresponding discontinuous internal phase is different. If the silicone acrylic hybrid pressure-sensitive adhesive is provided in n-heptane, the composition contains a continuous, silicone external phase and a discontinuous, acrylic internal phase. If the silicone acrylic hybrid pressure-sensitive adhesive is provided in ethyl acetate, the composition contains a continuous, acrylic external phase and a discontinuous, silicone internal phase. After evaporating the solvent in which the silicone acrylic hybrid pressure-sensitive adhesive is provided, the phase arrangement of the resulting pressure-sensitive adhesive film or layer corresponds to the phase arrangement of the solvent-containing adhesive coating composition. For example, in the absence of any substance that may induce an inversion of the phase arrangement in a silicone acrylic hybrid pressure sensitive adhesive composition, a pressure-sensitive adhesive layer prepared from a silicone acrylic hybrid pressure-sensitive adhesive in n-heptane provides a continuous, silicone external phase and a discontinuous, acrylic internal phase, a pressure-sensitive adhesive layer prepared from a silicone acrylic hybrid pressure-sensitive adhesive in ethyl acetate provides a continuous, acrylic external phase and a discontinuous, silicone internal phase. The phase arrangement of the compositions can, for example, be determined in peel force tests with pressure-sensitive adhesive films or layers prepared from the silicone acrylic hybrid PSA compositions which are attached to a siliconized release liner. The pressure-sensitive adhesive film contains a continuous, silicone external phase if the siliconized release liner cannot or can only hardly be removed from the pressure-sensitive adhesive film (laminated to a backing film) due to the blocking of the two silicone surfaces. Blocking results from the adherence of two silicone layers which comprise a similar surface energy. The silicone adhesive shows a good spreading on the siliconized liner and therefore can create a good adhesion to the liner. If the siliconized release liner can easily be removed the pressure-sensitive adhesive film contains a continuous, acrylic external phase. The acrylic adhesive has no good spreading due to the different surface energies and thus has a low or almost no adhesion to the siliconized liner.

According to a preferred embodiment of the invention the silicone acrylic hybrid polymer is a silicone acrylic hybrid pressure-sensitive adhesive obtainable from a silicon-containing pressure-sensitive adhesive composition comprising acrylate or methacrylate functionality. It is to be understood that the silicon-containing pressure-sensitive adhesive composition comprising acrylate or methacrylate functionality can include only acrylate functionality, only methacrylate functionality, or both acrylate functionality and methacrylate functionality.

According to certain embodiments of the invention the silicone acrylic hybrid pressure-sensitive adhesive comprises the reaction product of (a) a silicon-containing pressure-sensitive adhesive composition comprising acrylate or methacrylate functionality, (b) an ethylenically unsaturated monomer, and (c) an initiator. That is, the silicone acrylic hybrid pressure-sensitive adhesive is the product of the chemical reaction between these reactants ((a), (b), and (c)). In particular, the silicone acrylic hybrid pressure-sensitive adhesive includes the reaction product of (a) a silicon-containing pressure-sensitive adhesive composition comprising acrylate or methacrylate functionality, (b) a (meth)acrylate monomer, and (c) an initiator (i.e., in the presence of the initiator). That is, the silicone acrylic hybrid pressure-sensitive adhesive includes the product of the chemical reaction between these reactants ((a), (b), and (c)).

The reaction product of (a) a silicon-containing pressure-sensitive adhesive composition comprising acrylate or methacrylate functionality, (b) an ethylenically unsaturated monomer, and (c) an initiator may contain a continuous, silicone external phase and a discontinuous, acrylic internal phase or the reaction product of (a), (b), and (c) may contain a continuous, acrylic external phase and a discontinuous, silicone internal phase.

The silicon-containing pressure-sensitive adhesive composition comprising acrylate or methacrylate functionality (a) is typically present in the silicone acrylic hybrid pressure-sensitive adhesive in an amount of from 5 to 95, more typically 25 to 75, parts by weight based on 100 parts by weight of the hybrid pressure-sensitive adhesive.

The ethylenically unsaturated monomer (b) is typically present in the silicone acrylic hybrid pressure-sensitive adhesive in an amount of from 5 to 95, more typically 25 to 75, parts by weight based on 100 parts by weight of the hybrid pressure-sensitive adhesive.

The initiator (c) is typically present in the silicone acrylic hybrid pressure-sensitive adhesive in an amount of from 0.005 to 3, more typically from 0.01 to 2, parts by weight based on 100 parts by weight of the hybrid pressure-sensitive adhesive.

According to certain embodiments of the invention the silicon-containing pressure-sensitive adhesive composition comprising acrylate or methacrylate functionality (a) comprises the condensation reaction product of (a1) a silicone resin, (a2) a silicone polymer, and (a3) a silicon-containing capping agent which provides said acrylate or methacrylate functionality.

According to certain embodiments of the invention the silicon-containing pressure-sensitive adhesive composition comprising acrylate or methacrylate functionality (a) comprises the condensation reaction product of:
(a1) a silicone resin,
(a2) a silicone polymer, and
(a3) a silicon-containing capping agent which provides said acrylate or methacrylate functionality, wherein said silicon-containing capping agent is of the general formula XYR′_{b}SiZ_{3-b}, wherein
   X is a monovalent radical of the general formula AE-
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer.

According to certain embodiments of the invention the silicon-containing pressure-sensitive adhesive composition comprising acrylate or methacrylate functionality comprises the condensation reaction product of a pressure sensitive adhesive and a silicon-containing capping agent which provides said acrylate or methacrylate functionality. That is, the silicon-containing pressure sensitive adhesive composition comprising acrylate or methacrylate functionality is essentially a pressure sensitive adhesive that has been capped or end blocked with the silicon-containing capping agent which provides said acrylate or methacrylate functionality, wherein the pressure sensitive adhesive comprises the condensation reaction product of the silicone resin and the silicone polymer. Preferably, the silicone resin reacts in an amount of from 30 to 80 parts by weight to form the pressure sensitive adhesive, and the silicone polymer reacts in an amount of from 20 to 70 parts by weight to form the pressure sensitive adhesive. Both of these parts by weight are based on 100 parts by weight of the pressure sensitive adhesive. Although not required, the pressure sensitive adhesive may comprise a catalytic amount of a condensation catalyst. A wide array of silicone resins and silicone polymers are suitable to make up the pressure sensitive adhesive.

According to certain embodiments of the invention the silicone acrylic hybrid pressure-sensitive adhesive is the reaction product of:

(a) a silicon-containing pressure-sensitive adhesive composition comprising acrylate or methacrylate functionality that comprises the condensation reaction product of:
(a1) a silicone resin,
(a2) a silicone polymer, and
(a3) a silicon-containing capping agent which provides said acrylate or methacrylate functionality, wherein said silicon-containing capping agent is of the general formula XYR′_{b}SiZ_{3-b}, wherein
   X is a monovalent radical of the general formula AE-
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (b) an ethylenically unsaturated monomer; and
      (c) an initiator.

The silicone acrylic hybrid composition used in the present invention may be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE-
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in the presence of an initiator to form a silicone acrylic hybrid composition, optionally at a temperature of from 50° C. to 100° C., or from 65° C. to 90° C.

During the polymerization of the ethylenically unsaturated monomer and the silicon-containing pressure-sensitive adhesive composition comprising acrylate or methacrylate functionality, the silicone to acrylic ratio can be controlled and optimized as desired. The silicone to acrylic ratio can be controlled by a wide variety of mechanisms in and during the method. An illustrative example of one such mechanism is the rate controlled addition of the ethylenically unsaturated monomer or monomers to the silicon-containing pressure-sensitive adhesive composition comprising acrylate or methacrylate functionality. In certain applications, it may be desirable to have the silicone-based sub-species, or the overall silicone content, to exceed the acrylate-based sub-species, or the overall acrylic content. In other applications, it may be desirable for the opposite to be true. Independent of the end application, it is generally preferred, as already described above, that the silicon-containing pressure-sensitive adhesive composition comprising acrylate or methacrylate functionality is preferably present in the silicone acrylic hybrid composition in an amount of from about 5 to about 95, more preferably from about 25 to about 75, and still more preferably from about 40 to about 60 parts by weight based on 100 parts by weight of the silicone acrylic hybrid composition.

According to a certain embodiment of the invention, the silicone acrylic hybrid composition used in the present invention may be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE-
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in a first solvent in the presence of an initiator at a temperature of from 50° C. to 100° C. to form a silicone acrylic hybrid composition;
      (iii) removing the first solvent; and
      (iv) adding a second solvent to form the silicone acrylic hybrid composition, wherein the phase arrangement of the silicone acrylic hybrid composition is selectively controlled by selection of the second solvent.

The silicone acrylic hybrid PSA composition used in the present invention may also be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE-
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in a first solvent in the presence of an initiator at a temperature of from 50° C. to 100° C. to form a silicone acrylic hybrid composition;
      (iii) adding a processing solvent, wherein the processing solvent has a higher boiling point than the first solvent, and
      (iv) applying heat at a temperature of from 70° C. to 150° C. such that a majority of the first solvent is selectively removed;
      (v) removing the processing solvent; and.
      (vi) adding a second solvent to form the silicone acrylic hybrid composition, wherein the phase arrangement of the silicone acrylic hybrid composition is selectively controlled by selection of the second solvent.

The silicone resin according to the previous paragraphs may contain a copolymer comprising triorganosiloxy units of the formula R^{X}₃SiO_{1/2} and tetrafunctional siloxy units of the formula SiO_{4/2} in a ratio of from 0.1 to 0.9, preferably of about 0.6 to 0.9, triorganosiloxy units for each tetrafunctional siloxy unit. Preferably, each R^{X} independently denotes a monovalent hydrocarbon radical having from 1 to 6 carbon atoms, vinyl, hydroxyl or phenyl groups.

The silicone polymer according to the previous paragraphs may comprise at least one polydiorganosiloxane and is preferably end-capped (end-blocked) with a functional group selected from the group consisting of hydroxyl groups, alkoxy groups, hydride groups, vinyl groups, or mixtures thereof. The diorganosubstituent may be selected from the group consisting of dimethyl, methylvinyl, methylphenyl, diphenyl, methylethyl, (3,3,3-trifluoropropyl)methyl and mixtures thereof. Preferably, the diorganosubstituents contain only methyl groups. The molecular weight of polydiorganosiloxane will typically range from about 50,000 to about 1,000,000, preferably, from about 80,000 to about 300,000. Preferably, the polydiorganosiloxane comprises AR^{X}SiO units terminated with endblocking TR^{X}ASiO_{1/2} units, wherein the polydiorganosiloxane has a viscosity of from about 100 centipoise to about 30,000,000 centipoise at 25° C., each A radical is independently selected from R^{X} or halohydrocarbon radicals having from 1 to 6 carbon atoms, each T radical is independently selected from the group consisting of R^{X}, OH, H or OR^{Y}, and each R^{Y} is independently an alkyl radical having from 1 to 4 carbon atoms.

As an example using forms of the preferred silicone resin and the preferred silicone polymer, one type of pressure sensitive adhesive is made by:

mixing (i) from 30 to 80 inclusive parts by weight of at least one resin copolymer containing silicon-bonded hydroxyl radicals and consisting essentially of R^{X}₃SiO_{1/2} units and SiO_{4/2} units in a mole ratio of 0.6 to 0.9 R^{X}₃SiO_{1/2} units for each SiO_{4/2} unit present, (ii) between about 20 and about 70 parts by weight of at least one polydiorganosiloxane comprising AR^{X}SiO units terminated with endblocking TR^{X}ASiO_{1/2} units, wherein the polydiorganosiloxane has a viscosity of from about 100 centipoise to about 30,000,000 centipoise at 25° C. and each R^{X} is a monovalent organic radical selected from the group consisting of hydrocarbon radicals of from 1 to 6 inclusive carbon atoms, each A radical is independently selected from R^{X} or halohydrocarbon radicals having from 1 to 6 inclusive carbon atoms, each T radical is independently selected from the group consisting of R^{X}, OH, H or OR^{Y}, and each R^{Y} is independently an alkyl radical of from 1 to 4 inclusive carbon atoms; a sufficient amount of (iii) at least one of the silicon-containing capping agents, also referred to throughout as endblocking agents, described below and capable of providing a silanol content, or concentration, in the range of 5,000 to 15,000, more typically 8,000 to 13,000, ppm, when desirable an additional catalytic amount of (iv) a mild silanol condensation catalyst in the event that none is provided by (ii), and when necessary, an effective amount of (v) an organic solvent which is inert with respect to (i), (ii), (iii) and (iv) to reduce the viscosity of a mixture of (i), (ii), (iii), and (iv), and condensing the mixture of (i), (ii), (iii) and (iv) at least until a substantial amount of the silicon-containing capping agent or agents have reacted with the silicon-bonded hydroxyl radicals and T radicals of (i) and (ii). Additional organosilicon endblocking agents can be used in conjunction with the silicon-containing capping agent or agents (iii) of the present invention.

The silicon-containing capping agent according to the previous paragraphs may be selected from the group of acrylate functional silanes, acrylate functional silazanes, acrylate functional disilazanes, acrylate functional disiloxanes, methacrylate functional silanes, methacrylate functional silazanes, methacrylate functional disilazanes, meth-acrylate functional disiloxanes, and combinations thereof and may be described as to be of the general formula XYR′_{b}SiZ_{3-b}, wherein X is a monovalent radical of the general formula AE- where E is —O— or —NH— and A is an acryl group or a methacryl group, Y is a divalent alkylene radical having from 1 to 6 carbon atoms, R′ is a methyl or a phenyl radical, Z is a monovalent hydrolyzable organic radical or a halogen, and b is 0, 1 or 2. Preferably, the monovalent hydrolyzable organic radical is of the general formula R″O— where R″ is an alkylene radical. Most preferably, this particular endblocking agent is selected from the group of 3-methacryloxypropyldimethylchlorosilane, 3-methacryloxypropyldichlorosilane, 3-methacryloxypropyltrichlorosilane, 3-methacryloxypropyldimethylmethoxysilane, 3-methacryloxypropylmethyldimethoxysilane, 3-methacryloxypropyltrimethoxysilane, 3-methacryloxypropyldimethylethoxysilane, 3-methacryloxypropylmethyldiethoxysilane, 3-methacryloxypropyltriethoxysilane, (methacryloxymethyl)dimethylmethoxysilane, (methacryloxymethyl)methyldimethoxysilane, (methacryloxymethyl)trimethoxysilane, (methacryloxymethyl)dimethylethoxysilane, (methacryloxymethyl)methyldiethoxysilane, methacryloxymethyltriethoxysilane, methacryloxy-propyltriisopropoxysilane, 3-methacryloxypropyldimethylsilazane, 3-acryloxy-propyldimethylchlorosilane, 3-acryloxypropyldichlorosilane, 3-acryloxypropyl-trichlorosilane, 3-acryloxypropyldimethylmethoxysilane, 3-acryloxy-propylmethyldimethoxysilane, 3-acryloxypropyltrimethoxysilane, 3-acryloxypropyl-dimethylsilazane, and combinations thereof.

The ethylenically unsaturated monomer according to the previous paragraphs can be any monomer having at least one carbon-carbon double bond. Preferably, the ethylenically unsaturated monomer according to the previous paragraphs may be a compound selected from the group consisting of aliphatic acrylates, aliphatic methacrylates, cycloaliphatic acrylates, cycloaliphatic methacrylates, and combinations thereof. It is to be understood that each of the compounds, the aliphatic acrylates, the aliphatic methacrylates, the cycloaliphatic acrylates, and the cycloaliphatic methacrylates, include an alkyl radical. The alkyl radicals of these compounds can include up to 20 carbon atoms. The aliphatic acrylates that may be selected as one of the ethylenically unsaturated monomers are selected from the group consisting of methyl acrylate, ethyl acrylate, propyl acrylate, n-butyl acrylate, iso-butyl acrylate, tert-butyl acrylate, hexyl acrylate, 2-ethylhexyl acrylate, iso-octyl acrylate, iso-nonyl acrylate, iso-pentyl acrylate, tridecyl acrylate, stearyl acrylate, lauryl acrylate, and mixtures thereof. The aliphatic methacrylates that may be selected as one of the ethylenically unsaturated monomers are selected from the group consisting of methyl methacrylate, ethyl methacrylate, propyl methacrylate, n-butyl methacrylate, iso-butyl meth-acrylate, tert-butyl methacrylate, hexyl methacrylate, 2-eth-ylhexyl methacrylate, iso-octyl methacrylate, iso-nonyl methacrylate, iso-pentyl methacrylate, tridecyl methacrylate, stearyl methacrylate, lauryl methacrylate, and mixtures thereof. The cycloaliphatic acrylate that may be selected as one of the ethylenically unsaturated monomers is cyclohexyl acrylate, and the cycloaliphatic methacrylate that may be selected as one of the ethylenically unsaturated monomers is cyclohexyl methacrylate.

It is to be understood that the ethylenically unsaturated monomer used for preparing the silicone acrylic hybrid pressure sensitive adhesive may be more than one ethylenically unsaturated monomer. That is, a combination of ethylenically unsaturated monomers may be polymerized, more specifically co-polymerized, along with the silicon-containing pressure sensitive adhesive composition comprising acrylate or methacrylate functionality and the initiator. According to a certain embodiment of the invention, the silicone acrylic hybrid pressure-sensitive adhesive is prepared by using at least two different ethylenically unsaturated monomers, preferably selected from the group of 2-ethylhexyl acrylate and methyl acrylate, preferably in a ratio of from 40:60 to 70:30, more preferably in a ratio of from 65:35 to 55:45 or of from 55:45 to 45:50, particular preferred in a ratio of 50% 2-ethylhexyl acrylate and 50% methyl acrylate, or in a ratio of 60% 2-ethylhexyl acrylate and 40% methyl acrylate, as the acrylic monomer.

The initiator according to the previous paragraphs may be any substance that is suitable to initiate the polymerization of the silicon-containing pressure sensitive adhesive composition comprising acrylate or methacrylate functionality and the ethylenically unsaturated monomer to form the silicone acrylic hybrid. For example, free radical initiators selected from the group of peroxides, azo compounds, redox initiators, and photo-initiators may be used.

Further suitable silicone resins, silicone polymers, silicon-containing capping agents, ethylenically unsaturated monomers, and initiators that can be used in accordance with the previous paragraphs are detailed in WO 2007/145996, EP 2 599 847 A1, and WO 2016/130408.

According to a certain embodiment of the invention, the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.

According to a certain other embodiment of the invention, the silicone acrylic hybrid polymer comprises a reaction product of a silicone polymer, a silicone resin and an acrylic polymer, wherein the silicone resin contains triorganosiloxy units R₃SiO_{1/2} where R is an organic group, and tetrafunctional siloxy units SiO_{4/2} in a mole ratio of from 0.1 to 0.9 R₃SiO_{1/2} units for each SiO_{4/2}.

The acrylic polymer may comprise at least an alkoxysilyl functional monomer, polysiloxane-containing monomer, halosilyl functional monomer or alkoxy halosilyl functional monomer. Preferably, the acrylic polymer is prepared from alkoxysilyl functional monomers selected from the group consisting of trialkoxylsilyl (meth)acrylates, dialkoxyalkylsilyl (meth)acrylates, and mixtures thereof, or comprises end-capped alkoxysilyl functional groups. The alkoxysilyl functional groups may preferably be selected from the group consisting of trimethoxylsilyl groups, dimethoxymethylsilyl groups, triethoxylsilyl, diethoxymethylsilyl groups and mixtures thereof.

The acrylic polymer may also be prepared from a mixture comprising polysiloxane-containing monomers, preferably from a mixture comprising polydimethylsiloxane mono (meth)acrylate.

The silyl functional monomers will typically be used in amounts of from 0.2 to 20 weight percent of the acrylic polymer, more preferably the amount of silyl functional monomers will range from about 1.5 to about 5 weight percent of the acrylic polymer.

The amount of polysiloxane-containing monomer will typically be used in amounts of from 1.5 to 50 weight percent of the acrylic polymer, more preferably the amount of polysiloxane-containing monomers will range from 5 to 15 weight percent of the acrylic polymer.

Alternatively, the acrylic polymer comprises a block or grafted copolymer of acrylic and polysiloxane. An example of a polysiloxane block copolymer is polydimethylsiloxane-acrylic block copolymer. The preferred amount of siloxane block is 10 to 50 weight percent of the whole block polymer.

The acrylic polymer comprises alkyl (meth)acrylate monomers. Preferred alkyl (meth)acrylates which may be used have up to about 18 carbon atoms in the alkyl group, preferably from 1 to about 12 carbon atoms in the alkyl group. Preferred low glass transition temperature (Tg) alkyl acrylate with a homopolymer Tg of less than about 0° C. have from about 4 to about 10 carbon atoms in the alkyl group and include butyl acrylate, amyl acrylate, hexyl acrylate, 2-ethylhexyl acrylate, octyl acrylate, isooctyl acrylate, decyl acrylate, isomers thereof, and combinations thereof. Particularly preferred are butyl acrylate, 2-ethylhexyl acrylate and isooctyl acrylate. The acrylic polymer components may further comprise (meth)acrylate monomers having a high Tg such as methyl acrylate, ethyl acrylate, methyl methacrylate and isobutyl methacrylate.

The acrylic polymer component may further comprise a polyisobutylene group to improve cold flow properties of the resultant adhesive.

The acrylic polymer components may comprise nitrogen-containing polar monomers. Examples include N-vinyl pyrrolidone, N-vinyl caprolactam, N-tertiary octyl acrylamide, dimethyl acrylamide, diacetone acrylamide, N-tertiary butyl acrylamide, N-isopropyl acrylamide, cyanoethylacrylate, N-vinyl acetamide and N-vinyl formamide.

The acrylic polymer component may comprise one or more hydroxyl containing monomers such as 2-hydroxyethyl acrylate, 2-hydroxyethyl methacrylate, hydroxypropyl acrylate and/or hydroxypropyl methacrylate.

The acrylic polymer components may, if desired, comprise carboxylic acid containing monomers. Useful carboxylic acids preferably contain from about 3 to about 6 carbon atoms and include, among others, acrylic acid, methacrylic acid, itaconic acid, β-carboxyethyl acrylate and the like. Acrylic acid is particularly preferred.

Other useful, well known co-monomers include vinyl acetate, styrene, cyclohexyl acrylate, alkyl di(meth)acrylates, glycidyl methacrylate and allyl glycidyl ether, as well as macromers such as, for example, poly(styryl)methacrylate.

One acrylic polymer component that can be used in the practice of the invention is an acrylic polymer that comprises from about 90 to about 99.5 wt % of butyl acrylate and from about 0.5 to about 10 wt % dimethoxymethylsilyl methacrylate.

According to a certain embodiment of the invention the silicone acrylic hybrid polymer may be prepared by a) reacting silicone polymer with silicone resin to form a resultant product, b) reacting the resultant product of a) with an acrylic polymer containing reactive functionality, wherein the components are reacted in an organic solvent.

According to a certain embodiment of the invention the silicone acrylic hybrid polymer may be prepared by a) reacting a silicone resin with an acrylic polymer containing reactive functionality to form a resultant product, b) reacting the resultant product of a) with silicone polymer, wherein the components are reacted in an organic solvent.

According to a certain embodiment of the invention the silicone acrylic hybrid polymer may be prepared by a) reacting a silicone polymer with an acrylic polymer containing reactive functionality to form a resultant product, b) reacting the resultant product of a) with silicone resin, wherein the components are reacted in an organic solvent.

Further suitable acrylic polymers, silicone resins, and silicone polymers that can be used for chemically reacting together a silicone polymer, a silicone resin and an acrylic polymer to provide a silicone acrylic hybrid polymer in accordance with the previous paragraphs are detailed in WO 2010/124187.

According to certain embodiments of the invention, the silicone acrylic hybrid polymer used in the TTS is blended with one or more non-hybrid polymers, preferably the silicone acrylic hybrid polymer is blended with one or more non-hybrid pressure sensitive adhesives (e.g. pressure-sensitive adhesives based on polysiloxanes or acrylates).

### Non-Hybrid Polymers

According to a certain embodiment of the invention, the TTS comprises one or more non-hybrid polymers (e.g. non-hybrid pressure-sensitive adhesives) in addition to the silicone acrylic hybrid polymer. Non-hybrid polymers (e.g. non-hybrid pressure-sensitive adhesives) are polymers (e.g. polymer-based pressure-sensitive adhesives) which do not include a hybrid species. Preferred are non-hybrid polymers (e.g. non-hybrid pressure-sensitive adhesives) based on polysiloxanes, acrylates, polyisobutylenes, or styrene-isoprene-styrene block copolymers.

In certain embodiments, at least one non-hybrid polymer (e.g. at least one non-hybrid pressure-sensitive adhesive) is contained in the active agent containing layer in addition to the silicone acrylic hybrid polymer. At least one non-hybrid polymer may additionally be contained in the skin contact layer.

The non-hybrid polymers (e.g. the non-hybrid pressure-sensitive adhesives) may be contained in the active agent-containing layer structure and in the adhesive overlay.

Non-hybrid pressure-sensitive adhesives are usually supplied and used in solvents like n-heptane and ethyl acetate. The solids content of the pressure-sensitive adhesives is usually between 30% and 80%.

Suitable non-hybrid polymers according to the invention are commercially available e.g. under the brand names Bio-PSAs (polysiloxanes), Oppanol™ (polyisobutylenes), JSR-SIS (a styrene-isoprene-styrene copolymer) or Duro-Tak™ (acrylic polymers).

Polymers based on polysiloxanes may also be referred to as silicone-based polymers, or polysiloxane-based polymers. Pressure-sensitive adhesives based on polysiloxanes may also be referred to as silicone-based pressure-sensitive adhesives, or polysiloxane-based pressure-sensitive adhesives. Pressure-sensitive adhesives based on polysiloxanes may have a solids content preferably between 60% and 80%. Such silicone-based PSAs need, unlike other organic pressure sensitive adhesives, no additives like antioxidants, stabilizers, plasticizers, catalysts or other potentially extractable ingredients. These pressure-sensitive adhesives provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. It is possible to provide them with sufficient amine resistance and therefore enhanced stability in the presence of amines. Such pressure-sensitive adhesives are based on a resin-in-polymer concept wherein, by condensation reaction of silanol end blocked polydimethylsiloxane with a silica resin, a polysiloxane is prepared which for amine stability the residual silanol functionality is additionally capped with trimethylsiloxy groups. The silanol end blocked polydimethylsiloxane content contributes to the viscous component of the visco-elastic behavior, and impacts the wetting and the spreadability properties of the adhesive. The resin acts as a tackifying and reinforcing agent, and participates in the elastic component. The correct balance between silanol end blocked polydimethylsiloxane and resin provides for the correct adhesive properties.

Examples of silicone-based PSA compositions which are commercially available include the standard BIO-PSA series (7-4400, 7-4500 and 7-4600 series) and the amine compatible (endcapped) BIO-PSA series (7-4100, 7-4200 and 7-4300 series), typically supplied in n-heptane or ethyl acetate by Dow Corning. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPa s and a complex viscosity at 0.01 rad/s at 30° C. of 5×10⁶ Poise.

The pressure-sensitive adhesives based on polysiloxanes are supplied and used in solvents like n-heptane, ethyl acetate or other volatile silicone fluids. The solids content of pressure-sensitive adhesives based on polysiloxanes in solvents is usually between 60 and 85%, preferably between 70 and 80%. The skilled person is aware that the solids content may be modified by adding a suitable amount of solvent.

The preferred pressure-sensitive adhesives based on polysiloxanes in accordance with the invention are characterized by a solution viscosity at 25° C. and 60% solids content in n-heptane of more than about 150 mPa s, or from about 200 mPa s to about 700 mPa s, or of about 450 mPa s or of about 500 mPa s, preferably as measured using a Brookfield RVT viscometer equipped with a spindle number 5 at 50 rpm. Theses may also be characterized by a complex viscosity at 0.01 rad/s at 30° C. of less than about 1×10⁹ Poise or from about 1×10⁵ to about 9×10⁸ Poise, or of about 1×10⁸ Poise, or of about 5×10⁶ Poise, preferably as measured using a Rheometrics ARES rheometer, wherein the rheometer is equipped with 8 mm plates and the gap zeroed.

Suitable polyisobutylenes according to the invention are available under the tradename Oppanol®. Combinations of high-molecular weight polyisobutylenes (B100/B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, preferably from 95:5 to 40:60, more preferably from 90:10 to 80:20. A preferred example for a polyisobutylene combination is B10/B100 in a ratio of 85/15. Oppanol® B100 has a viscosity average molecular weight Mᵥ of 1,110,000, and a weight average molecular weight M_{w} of 1,550,000, and an average molecular weight distribution M_{w}/Mₙ of 2.9. Oppanol® B10 has a viscosity average molecular weight Mᵥ of 40,000, and a weight average molecular weight M_{w} of 53,000, and an average molecular weight distribution M_{w}/Mₙ of 3.2. In certain embodiments, polybutene may be added to the polyisobutylenes. The solids content of polyisobutylenes in solvents is usually between 30 and 50%, preferably between 35 and 40%. The skilled person is aware that the solids content may be modified by adding a suitable amount of solvent.

Pressure-sensitive adhesives based on acrylates may also be referred to as acrylate-based pressure-sensitive adhesives, or acrylate pressure-sensitive adhesives. Pressure-sensitive adhesives based on acrylates may have a solids content preferably between 30% and 60%. Such acrylate-based pressure-sensitive adhesives may or may not comprise functional groups such as hydroxy groups, carboxylic acid groups, neutralized carboxylic acid groups and mixtures thereof. Thus, the term “functional groups” in particular refers to hydroxy- and carboxylic acid groups, and deprotonated carboxylic acid groups.

Corresponding commercial products are available e.g. from Henkel under the tradename Duro Tak®. Such acrylate-based pressure-sensitive adhesives are based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate, and are provided in ethyl acetate, heptanes, n-heptane, hexane, methanol, ethanol, isopropanol, 2,4-pentanedione, toluene or xylene or mixtures thereof. Suitable acrylate-based pressure-sensitive adhesives are based on monomers selected from two or more of acrylic acid, butylacrylate, 2-ethylhexylacrylate, glycidylmethacrylate, 2-hydroxyethylacrylate, methylacrylate, methylmethacrylate, t-octylacrylamide and vinylacetate.

In one embodiment of the invention, the at least one non-hybrid polymer is an acrylate-based pressure-sensitive adhesive characterized by a solution viscosity at 25° C. and about 39% solids content in ethyl acetate of from about 4000 mPa s to about 12000 mPa s, preferably as measured using a e.g. Brookfield SSA, viscometer equipped with a spindle number 27 at 20 RPM.

Specific acrylate-based pressure-sensitive adhesives are available as:
Duro-Tak™ 87-4287 (a copolymer based on vinyl acetate, 2-ethylhexyl-acrylate, and 2-hydroxyethyl-acrylate provided as a solution in ethyl acetate without cross-linking agent),
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
Duro-Tak™ 87-4098 (a copolymer based on 2-ethylhexyl-acrylate and vinyl acetate, provided as a solution in ethyl acetate).

Additional polymers may also be added to enhance cohesion and/or adhesion.

Certain polymers in particular reduce the cold flow and are thus in particular suitable as additional polymer. A polymeric matrix may show a cold flow, since such polymer compositions often exhibit, despite a very high viscosity, the ability to flow very slowly. Thus, during storage, the matrix may flow to a certain extent over the edges of the backing layer. This is a problem with storage stability and can be prevented by the addition of certain polymers. A basic acrylate polymer (e.g. Eudragit® E100) may e.g. be used to reduce the cold flow. Thus, in certain embodiments, the matrix layer composition comprises additionally a basic polymer, in particular an amine-functional acrylate as e.g. Eudragit® E100. Eudragit® E100 is a cationic copolymer based on dimethylaminoethyl methacrylate, butyl methacrylate, and methyl methacrylate with a ratio of 2:1:1. The monomers are randomly distributed along the copolymer chain. Based on SEC method, the weight average molar mass (Mw) of Eudragit® E100 is approximately 47,000 g/mol.

### Release Characteristics

The TTS in accordance with the invention are designed for transdermally administering active agent to a patient, preferably to the systemic circulation, for a predefined extended period of time, e.g. for about 24 hours, for about 84 hours, or about 168 hours. Whether the permeation rate of the active agent is sufficient for a therapeutic effect can be determined by comparing the Franz diffusion cell permeation rates of a commercially available reference TTS including the same active agent (e.g. BuTrans® for buprenorphine, Nicorette® for nicotine, or Exelon® for rivastigmine) with the Franz diffusion cell permeation rates of the TTS in accordance with the invention.

In accordance with the invention, the skin permeation rates are measured in a Franz diffusion cell with dermatomed human skin with a thickness of 800 μm, with an intact epidermis, in accordance with the OECD Guideline (adopted Apr. 13, 2004), using a phosphate buffer solution pH 5.5 with 0.1% saline azide as antibacteriological agent at a temperature of 32±1° C. Absolute mean values obtained from different permeation studies can be compared by using the reference TTS (e.g. BuTrans®) as an internal standard.

Permeation rates using a EVA-membrane are measured in a Franz diffusion cell with a EVA-membrane with a thickness of 50 μm, in accordance with the EMA Guideline on quality of transdermal patches (adopted Oct. 23, 2014), using a phosphate buffer pH 5.5 with 0.1% sodium azide at a temperature of 32±1° C. Absolute mean values obtained from different permeation studies can be compared by using the reference TTS (e.g. Exelon®) as an internal standard.

In a certain embodiment, the TTS according to the invention provides a permeation rate of the active agent when measured in a comparable test with a commercial active agent reference transdermal therapeutic system that is therapeutically effective, preferably over 8 hours, 12 hours, 16 hours, 24 hours, 32 hours, 48 hours, 72 hours, 84 hours, 96 hours, or 168 hours.

In a certain embodiment, the TTS according to the invention provides a permeation rate of the buprenorphine when measured in a comparable test with a commercial buprenorphine reference TTS (e.g. BuTrans®) that is therapeutically effective, preferably over 48 hours, 72 hours, 84 hours, 96 hours, or 168 hours.

In a certain embodiment, the TTS according to the invention provides a permeation rate of the nicotine when measured in a comparable test with a commercial nicotine reference TTS (e.g. Nicorette®) that is therapeutically effective, preferably over 24 hours.

In a certain embodiment, the TTS according to the invention provides a permeation rate of the rivastigmine when measured in a comparable test with a commercial rivastigmine reference TTS (e.g. Exelon®) that is therapeutically effective, preferably over 24 hours.

In one embodiment, the TTS according to the invention provides a permeation rate of the active agent that is constant within 20% points over about the last two-thirds of the administration period, e.g. over the last 16 hours of a 24-hour administration period. The permeation rate is preferably constant within less than 19% points, less than 18% points, or less than 17% points, over about the last two-thirds of the administration period, e.g. over the last 16 hours of a 24-hour administration period.

For the purpose of determining whether the permeation rate is constant within 20% points in accordance with the present invention, the relative amendment of the cumulative permeation rate from a certain point of elapsed time, e.g. 8 hours, to the end of the administration period, e.g. 24 hours, is calculated by subtracting the cumulative permeation rate over the entire administration period, e.g. at 24 hours, from the cumulative permeation rate at a certain elapsed time, e.g. at 24 hours, and dividing the result by the calculated cumulative permeation rate at the certain elapsed time, e.g. at 24 hours.

In a certain embodiment, the TTS according to the invention provides a permeation rate of the active agent (e.g. buprenorphine) that is constant within 20% points over about the last two-thirds of the administration period, preferably over the last 4 days of a 7-day administration period, i.e. from hour 72 to hour 168, preferably as measured in a Franz diffusion cell with dermatomed human skin with a thickness of 800 μm, with an intact epidermis, in accordance with the OECD Guideline (adopted Apr. 13, 2004), using a phosphate buffer solution pH 5.5 with 0.1% saline azide as antibacteriological agent at a temperature of 32±1° C. The permeation rate is preferably constant within less than 19% points, less than 18% points, or less than 17% points, over about the last two-thirds of the administration period, e.g. from hour 72 to hour 168.

In a certain embodiment, the TTS according to the invention provides a permeation rate of the active agent (e.g. nicotine or rivastigmine) that is constant within 20% points over about the last two-thirds of the administration period, preferably over the last 16 hours of a 24-hour administration period, i.e. from hour 8 to hour 24, preferably as measured in a Franz diffusion cell with dermatomed human skin with a thickness of 800 μm, with an intact epidermis, in accordance with the OECD Guideline (adopted Apr. 13, 2004), using a phosphate buffer solution pH 5.5 with 0.1% saline azide as antibacteriological agent at a temperature of 32±1° C. The permeation rate is preferably constant within less than 19% points, less than 18% points, or less than 17% points, over about the last two-thirds of the administration period, e.g. from hour 8 to hour 24.

In a certain embodiment, the TTS according to the invention provides a permeation rate of the active agent (e.g. buprenorphine) that is constant within 20% points over about the last two-thirds of the administration period, preferably over the last 4 days of a 7-day administration period, i.e. from hour 72 to hour 168, preferably as measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide is used at a temperature of 32±1° C. The permeation rate is preferably constant within less than 19% points, less than 18% points, or less than 17% points, over about the last two-thirds of the administration period, e.g. from hour 72 to hour 168.

In one embodiment, the permeation rate of the active agent (e.g. rivastigmine or nicotine) is constant within 20% points over the last 16 hours of a 24-hour administration period, i.e. from hour 8 to hour 24, preferably as measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide is used at a temperature of 32±1° C. The permeation rate is preferably constant within less than 19% points, less than 18% points, or less than 17% points, over about the last two-thirds of the administration period, e.g. from hour 8 to hour 24.

### Method of Treatment/Medical Use

In accordance with a specific aspect of the present invention, the TTS according to the invention is for use in a method of treating a human patient.

The method comprises the application of the TTS according to the invention the invention on the skin of a patient, in particular for about 24 hours, for at least 24 hours, for more than 3 days, for about 3.5 days, for about 4 days, about 5 days, about 6 days, or for about 7 days.

According to certain aspects, the TTS according to the invention is for use in a method of treating pain. In this connection, the TTS preferably comprises a therapeutically effective amount of buprenorphine and is preferably applied for more than 3 days, e.g. for about 3.5 days, particularly preferred for about 7 days (about 168 hours, or one week) to the skin of a patient.

According to one aspect, the present invention relates to a method of treating pain by applying to the skin of a patient a transdermal therapeutic system as described herein for about 24 hours, for at least 24 hours, for more than 3 days, for about 3.5 days, for about 4 days, about 5 days, about 6 days, or for about 7 days. In this connection, the TTS preferably comprises a therapeutically effective amount of buprenorphine and is preferably applied for more than 3 days, e.g. for about 3.5 days, particularly preferred for about 7 days (about 168 hours, or one week) to the skin of a patient.

According to certain aspects, the TTS according to the invention is for use in a method of treating nicotine addiction, in a method of smoking cessation treatment, in a method of treating Parkinson's disease or in a method of treating Alzheimer's disease. In this connection, the TTS preferably comprises a therapeutically effective amount of nicotine and is preferably applied for about 24 hours (1 day) to the skin of a patient.

According to another aspect, the present invention relates to a method of treating nicotine addiction, a method of smoking cessation treatment, a method of treating Parkinson's disease or a method of treating Alzheimer's disease by applying to the skin of a patient a transdermal therapeutic system as described herein for about 24 hours, for at least 24 hours. In this connection, the TTS preferably comprises a therapeutically effective amount of nicotine and is preferably applied for about 24 hours (1 day) to the skin of a patient.

According to certain aspects, the TTS according to the invention is for use in a method of preventing, treating, or delaying of progression of Alzheimer's disease, dementia associated with Parkinson's disease, and/or symptoms of traumatic brain injury, or mild to moderate dementia caused by Alzheimer's or Parkinson's disease. In this connection, the TTS preferably comprises a therapeutically effective amount of rivastigmine and is preferably applied for about 24 hours (1 day) to the skin of a patient.

According to another aspect, the present invention relates to a method of preventing, treating, or delaying of progression of Alzheimer's disease, dementia associated with Parkinson's disease, and/or symptoms of traumatic brain injury, or mild to moderate dementia caused by Alzheimer's or Parkinson's disease by applying to the skin of a patient a transdermal therapeutic system as described herein for about 24 hours, for at least 24 hours. In this connection, the TTS preferably comprises a therapeutically effective amount of rivastigmine and is preferably applied for about 24 hours (1 day) to the skin of a patient.

According to one aspect, the invention relates to the use of a TTS according to the present invention for the manufacture of a medicament. In particular, the invention relates to the use of a TTS according to the present invention for the manufacture of a medicament for treating pain, treating nicotine addiction, smoking cessation treatment, treating Parkinson's disease treating Alzheimer's disease, or preventing, treating, or delaying of progression of Alzheimer's disease, dementia associated with Parkinson's disease, and/or symptoms of traumatic brain injury, or mild to moderate dementia caused by Alzheimer's or Parkinson's disease, which preferably is applied to the skin of a patient for at least 24 hours, for more than 3 days, for about 3.5 days, for about 4 days, about 5 days, about 6 days, or for about 7 days.

### Method of Manufacture

The invention further relates to a method of manufacture of a transdermal therapeutic system according to the invention comprising the steps of:
1) providing an active agent-containing coating composition comprising
   a) the active agent, and
   b) optionally a solvent,
2) coating the active agent-containing coating composition onto a film in an amount to provide the desired area weight,
3) drying the coated active agent-containing coating composition to provide the active agent-containing layer,
4) providing an additional skin contact layer by coating and drying an additional coating composition according to steps 2 and 3, wherein the film is a release liner,
5) laminating the adhesive side of the skin contact layer onto the adhesive side of the active agent-containing layer to provide an active agent-containing layer structure with the desired area of release,
6) punching the individual systems from the active agent-containing layer structure,
7) optionally adhering to the individual systems an active agent-free self-adhesive layer structure comprising also a backing layer and an active agent-free pressure-sensitive adhesive layer and which is larger than the individual systems of active agent-containing self-adhesive layer structure,
   wherein at least one silicone acrylic hybrid polymer composition is added to the active agent-containing coating composition in step 1 and to the additional coating composition in step 4.

In a preferred embodiment, the at least one silicone acrylic hybrid polymer composition is a silicone acrylic hybrid pressure-sensitive adhesive, preferably in ethyl acetate or n-heptane.

In one embodiment, the at least one silicone acrylic hybrid polymer in the active agent-containing coating composition in step 1) is different to the silicone acrylic hybrid polymer in the additional coating composition in step 4). In another embodiment, the at least one silicone acrylic hybrid polymer in the active agent-containing coating composition in step 1) is the same silicone acrylic hybrid polymer as in the additional coating composition in step 4).

In one embodiment, in step 1) a non-hybrid pressure-sensitive adhesive based on polysiloxanes in n-heptane or in ethyl acetate is added. In another embodiment, in step 1) a non-hybrid pressure-sensitive adhesive based on acrylates is added.

In one embodiment, the film in step 2) is a release liner, wherein the active agent-containing layer is laminated after step 3) to a backing layer, and wherein the release liner of step 2) is removed before step 5). In another embodiment, the film in step 2) is a backing layer.

In a further embodiment, in step 4) a non-hybrid pressure-sensitive adhesive based on polysiloxanes in n-heptane or in ethyl acetate is added. In yet another embodiment, in step 4) a non-hybrid pressure-sensitive adhesive based on acrylate is added.

In one embodiment, the active agent-containing coating composition of step 1) further comprises a carboxylic acid.

In one embodiment, the active agent-containing coating composition of step 1) further comprises an auxiliary polymer, preferably selected from the group consisting of alkyl methacrylate copolymers, amino alkyl methacrylate copolymers, methacrylic acid copolymers, methacrylic ester copolymers, ammonioalkyl methacrylate copolymers, polyvinylpyrrolidones, vinylpyrrolidone-vinyl acetate copolymers, polyvinyl caprolactam-polyvinyl acetate-polyethylene glycol copolymer, and mixtures thereof.

Drying is performed preferably at a temperature of from 20 to 90° C., more preferably from 30 to 80° C.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Comparative Example 1

The commercially available product BuTrans®, also known as Norspan®, is used as a reference TTS (Comp. 1). In particular, absolute mean values obtained from in vitro permeation studies (which may vary from study to study) can be compared by using BuTrans® as an internal standard. BuTrans® is a homogeneous matrix system based on polyacrylates having a coating weight of 80 g/m² and containing buprenorphine in an amount of 0.8 mg/cm² (API loading).

### Comparative Example 2

### Coating Composition

The formulation of the buprenorphine-containing coating compositions of Comparative Examples 2 is summarized in Table 1.1 below. The formulations are based on the weight percent.

TABLE 1.1

| | Comp. 2 | |
|---|---|---|
| Ingredient (Trade Name) | Amt [g] | Solids [%] |
| Buprenorphine base | 9.00 | 10 |
| Levulinic acid | 6.30 | 7 |
| Ethanol | 8.67 | — |
| Ascorbyl palmitate | 0.18 | 0.2 |
| Silicone acrylic hybrid PSA in n-heptane | 149.04 | 82.8 |
| Solids content of 50% by weight (SilAc- | | |
| PSA 7-6101 from Dow Corning Healthcare) | | |
| n-heptane | 0.22 | — |
| Total | 173.4 | 100.0 |

### Preparation of the API Coating Composition

In a 250 mL wide-neck glass, the buprenorphine base was suspended in levulinic acid, ethanol, ascorbyl palmitate and stirred until complete dissolution of buprenorphine. The silicone acrylic hybrid pressure-sensitive adhesive in the form of a mixture in n-heptane having a solids content of 50% by weight and n-heptane to adjust the solids content were added. The mixture was stirred until homogeneous to give a buprenorphine-containing adhesive mixture with 5.19% by weight of buprenorphine, with a solids content of 51.9%.

### Coating of the API Coating Composition

The buprenorphine-containing adhesive mixture was coated within less than 24 h after the buprenorphine-containing mixture was finished on an adhesively equipped foil (Scotchpak 1022 from 23M, which may function as release liner) using hand over knife lab coating equipment, using an erichson coater. The solvent was removed by drying in a first step at approx. room temperature (23±2° C.) for approx. 10 min, followed by a second drying step at approx. 75° C. for approx. 10 min.

The coating thickness was chosen such that removal of the solvents results in an area weight of the matrix layer of approx. 90 g/m². This results in 10% by weight of buprenorphine (API loading 0.9 mg/cm²), 7% by weight of levulinic acid, 0.2% by weight of ascorbyl palmitate, and 82.8% by weight of silicone acrylic hybrid pressure-sensitive adhesive in this matrix layer. The dried film was then laminated with a backing layer (polyethylenterephthalate (PET) foil 19 μm) to provide the buprenorphine-containing self-adhesive layer structure.

### Preparation of the TTS

The individual systems (TTS) were then punched out from the buprenorphine-containing self-adhesive layer structure. In specific embodiments a TTS as described above can be provided with an adhesive overlay, i.e. a further self-adhesive layer structure of larger surface area, preferably with rounded corners, comprising a pressure-sensitive adhesive matrix layer which is free of active ingredient and a preferably skin-colored backing layer. The TTSs are then punched out and sealed into pouches of the primary packaging material.

### Measurement of Adhesion Force

Adhesion force tests were performed with the TTS using a tensile strength testing machine. Prior testing the samples were equilibrated 24 hours under controlled conditions at approx. room temperature (23±2° C.) and approx. 50% rh (relative humidity). Further, the samples were cut into pieces with a fixed width of 25 mm and a suitable length. The first millimeters of the adhesively equipped foil was pulled off and a splicing tape is applied to the opened adhesive side of the buprenorphine-containing layer structure. Then, the adhesively foil was totally removed and the sample was placed with the adhesive surface in longitudinal direction onto the center of the cleaned testing plate (aluminum). The testing plate was fixed to the lower clamp of the tensile strength machine. The machine was adjusted to zero, the splicing tape was gripped into the upper clamp of the machine. The pull angle was set to 90°. After measurement of the adhesion force of three samples, the mean value of the adhesion force was calculated. The measurement value is based on units “N/sample width” [N/25 mm].

TABLE 1.2

| Adhesion force [N/25 mm] (n = 3) | | Ratio Comp. 2/ |
|---|---|---|
| | Comp. 1 | Comp. 1 (BuTrans ®) |
| Comp. 2 | (BuTrans ®) | of Adhesion force |
| 6.4 | 3.1 | 2.1 |

### Measurement of Tack

The Tack (the force which is required to separate an object from an adhesive surface after a short time of contact) tests were performed with the TTS in accordance with the Standard Test Method for Pressure-Sensitive Tack of Adhesives Using an Inverted Probe Machine (ASTM D 2979-01; Reapproved 2009) using a probe tack tester PT-1000 (ChemInstruments). Prior to testing the samples were equilibrated 24 hours under controlled conditions at approx. room temperature (23±2° C.) and approx. 50% rh. For determining the tack, the tip of a cleaned probe with a diameter of 5 mm was brought into contact with the adhesive surface of the buprenorphine-containing layer structure for 1 second, at a defined rate (10±0.1 mm/s), under defined pressure (9.79±0.10 kPa), at a given temperature (23±2° C.) and the bond formed between probe and the adhesive was subsequently broken at the same rate. Tack was measured as the maximum force required, to break the adhesion bond (see ASTM D2979-01; Reapproved 2009). After finalization the mean value from the individual results of three associated samples were calculated and the mean tack value reported in [N].

TABLE 1.3

| Tack [N] (n = 3) | | Ratio Comp. 2/ |
|---|---|---|
| | Comp. 1 | Comp. 1 (BuTrans ®) |
| Comp. 2 | (BuTrans ®) | of Tack |
| 0.88 | 1.19 | 0.7 |

### Measurement of Permeation

The permeated amount and the corresponding permeation rates of Comparative Examples 1 and 2 were determined by in vitro experiments in accordance with the OECD Guideline (adopted Apr. 13, 2004) carried out with a 9.0 ml Franz diffusion cell. Split thickness human skin from cosmetic surgeries (female abdomen, date of birth 1988) was used. A dermatome was used to prepare skin to a thickness of 800 μm, with an intact epidermis for all TTS. Due to the prolonged test (168 hours) 800 μm skin is used instead of the recommended 200 to 400 μm skin. Die cuts with an area of 1.191 cm² were punched from the TTS. The concentrations of buprenorphine base in the receptor medium of the Franz diffusion cell (phosphate buffer solution pH 5.5 with 0.1% saline azide as antibacteriological agent) at a temperature of 32±1° C. were measured and the corresponding permeation rate calculated.

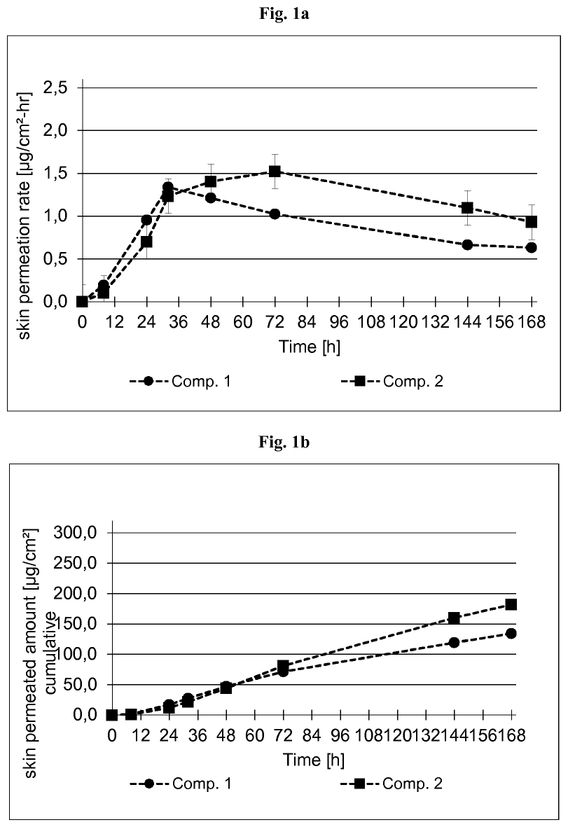

The results for Comparative Examples 1 and 2 are shown in Tables 1.4 to 1.8 and FIGS. 1a and 1b.

TABLE 1.4
Permeated amount with SD [μg/cm²]

| Elapsed | Comp. 2 (n = 3) | | Comp. 1 (n = 3) | |
|---|---|---|---|---|
| time [h] | Amount | SD | Amount | SD |
| 0 | 0 | 0 | 0 | 0 |
| 8 | 0.82 | 0.16 | 1.54 | 0.29 |
| 24 | 11.20 | 0.78 | 15.27 | 0.55 |
| 32 | 9.87 | 0.83 | 10.70 | 0.44 |
| 48 | 22.50 | 0.87 | 19.37 | 0.76 |
| 72 | 36.53 | 0.74 | 24.60 | 0.98 |
| 144 | 78.90 | 1.11 | 47.73 | 3.75 |
| 168 | 22.27 | 0.49 | 15.13 | 0.70 |
| Cum. | 182.1 | 4 | 134.3 | 7 |
| at 168 h | | | | |

TABLE 1.5
Permeation rate with SD [μg/cm²-h]

| Elapsed | Comp. 2 (n = 3) | | Comp. 1 (n = 3) | |
|---|---|---|---|---|
| time [h] | Rate | SD | Rate | SD |
| 0 | 0 | 0 | 0 | 0 |
| 8 | 0.10 | 0.02 | 0.19 | 0.04 |
| 24 | 0.70 | 0.05 | 0.95 | 0.03 |
| 32 | 1.23 | 0.10 | 1.34 | 0.05 |
| 48 | 1.41 | 0.05 | 1.21 | 0.05 |
| 72 | 1.52 | 0.03 | 1.03 | 0.04 |
| 144 | 1.10 | 0.02 | 0.66 | 0.05 |
| 168 | 0.93 | 0.02 | 0.63 | 0.03 |

TABLE 1.6
Cumulative permeation rate [μg/cm²-h]

| over 72 hours | | over 168 hours | |
|---|---|---|---|
| | Comp. 1 | | Comp. 1 |
| Comp. 2 | (BuTrans ®) | Comp. 2 | (BuTrans ®) |
| 1.1 | 1.0 | 1.1 | 0.8 |

TABLE 1.7
Ratio Cumulative permeated amount
after 168 hours of release/API
Loading (active agent utilization)

| | Comp. 1 |
|---|---|
| Comp. 2 | (BuTrans ®) |
| 0.20 | 0.17 |

TABLE 1.8

| Ratio Comp. 2/Comp. 1 | |
|---|---|
| (BuTrans ®) of cumulative | Ratio Comp. 2/Comp. 1 |
| permeated amount after | (BuTrans ®) of active |
| 168 hours of release | agent utilization |
| 1.4 | 1.2 |

### Examples 1A, 1B

### Coating Composition

The formulation of the buprenorphine-containing coating compositions of Examples 1a and 1b and the formulations of the active agent-free coating composition for the skin contact layers of Examples 1a and 1b are summarized in Table 2.1 below. The formulations are based on weight percent.

TABLE 2.1

| | Ex. 1a and Ex. 1b | |
|---|---|---|
| | API containing composition | |
| Ingredient (Trade Name) | Amt [g] | Solids [%] |
| Buprenorphine base | 9.00 | 10 |
| Levulinic acid | 6.30 | 7 |
| Ethanol | 8.67 | — |
| Ascorbyl palmitate | 0.18 | 0.2 |
| Silicone acrylic hybrid PSA in n-heptane | 149.04 | 82.8 |
| Solids content of 50% by weight (SilAc- | | |
| PSA 7-6101 from Dow Corning | | |
| Healthcare) | | |
| n-heptane | 0.22 | — |
| Total | 173.4 | 100.0 |
| | API free coating composition | |
| | for the skin contact layer | |
| | Solids [%] | |
| | Ex. 1a | Ex. 1b |
| Silicone acrylic hybrid PSA in n heptane | 100.0 | — |
| Solids content of 50% by weight (SilAc- | | |
| PSA 7-6301 from Dow Corning | | |
| Healthcare) | | |
| Silicone acrylic hybrid PSA in ethyl acetate | — | 100.0 |
| Solids content of 50% by weight (SilAc- | | |
| PSA 7-6302 from Dow Corning | | |
| Healthcare) | | |

### Preparation of the API Coating Composition

In a 250 mL wide-neck glass, the buprenorphine base was suspended in levulinic acid, ethanol, ascorbyl palmitate and stirred until complete dissolution of buprenorphine. The silicone acrylic hybrid pressure-sensitive adhesive in the form of a mixture in n-heptane having a solids content of 50% by weight and n-heptane to adjust the solids content were added. The mixture was stirred until homogeneous to give a buprenorphine-containing adhesive mixture with 5.19% by weight of buprenorphine, with a solids content of 51.9%.

### Coating of the API Coating Composition

The buprenorphine-containing adhesive mixture was coated within less than 24 h after the buprenorphine-containing mixture was finished on an adhesively equipped foil (Scotchpak 1022 from 23M, which may function as release liner) using hand over knife lab coating equipment, using an erichson coater. The solvent was removed by drying in a first step at approx. room temperature (23±2° C.) for approx. 10 min, followed by a second drying step at approx. 75° C. for approx. 10 min.

The coating thickness was chosen such that removal of the solvents results in an area weight of the matrix layer of approx. 90 g/m². This results in 10% by weight of buprenorphine, 7% by weight of levulinic acid, 0.2% by weight of ascorbyl palmitate, and 82.8% by weight of silicone acrylic hybrid pressure-sensitive adhesive in this matrix layer. The dried film was then laminated with a backing layer (polyethylenterephthalate (PET) foil 19 μm).

### Coating of the API Free Coating Composition (Skin Contact Layer) and Lamination

The active agent-free coating composition was coated on an adhesively equipped foil using hand over knife lab coating equipment (erichson coater).

The coating thickness was each chosen such that removal of the solvents result in an area weight of the skin contact layer of approx. 20 g/m². This results in 100% by weight of silicone acrylic hybrid pressure-sensitive adhesive in this skin contact layer.

The dried film was then laminated with the buprenorphine-containing matrix layer that was laminated with a backing layer. For this purpose, the adhesively equipped foil used for the coating and drying of the buprenorphine-containing matrix layer that was then laminated with a backing layer was removed and the coated and dried buprenorphine free skin contact layer was laminated with that film resulting in a buprenorphine-containing self-adhesive layer structure.

TABLE 2.2

| | Ex. 1a | Ex. 1b |
|---|---|---|
| Area Weight API containing matrix [g/m²] | 90 | 90 |
| Area Weight skin contact layer [g/m²] | 20 | 20 |
| API Loading [mg/cm²] | 0.9 | 0.9 |

### Preparation of the TTS (Concerning all Examples)

The individual systems (TTS) were then punched out from the active agent-containing self-adhesive layer structure. In specific embodiments a TTS as described above can be provided with an adhesive overlay, i.e. a further self-adhesive layer structure of larger surface area, preferably with rounded corners, comprising a pressure-sensitive adhesive matrix layer which is free of active ingredient and a preferably skin-colored backing layer. The TTSs are then punched out and sealed into pouches of the primary packaging material.

### Measurement of Adhesion Force

Adhesion force tests were performed with the TTS using a tensile strength testing machine. Prior testing the samples were equilibrated 24 hours under controlled conditions at approx. room temperature (23±2° C.) and approx. 50% rh (relative humidity). Further, the samples were cut into pieces with a fixed width of 25 mm and a suitable length. The first millimeters of the adhesively equipped foil was pulled off and a splicing tape is applied to the opened adhesive side of the buprenorphine-containing layer structure. Then, the adhesively foil was totally removed and the sample was placed with the adhesive surface in longitudinal direction onto the center of the cleaned testing plate (aluminum). The testing plate was fixed to the lower clamp of the tensile strength machine. The machine was adjusted to zero, the splicing tape was gripped into the upper clamp of the machine. The pull angle was set to 90°. After measurement of the adhesion force of three samples, the mean value of the adhesion force was calculated. The measurement value is based on units “N/sample width” [N/25 mm].

TABLE 2.3
Adhesion force [N/25 mm]

| Ex. 1a (n = 3) | Ex. 1b (n = 1) | Comp. 1 (n = 3) |
|---|---|---|
| 4.9 | 15.8* | 3.1 |

*partially cohesion fracture

### Measurement of Tack

The Tack (the force which is required to separate an object from an adhesive surface after a short time of contact) tests were performed with the TTS in accordance with the Standard Test Method for Pressure-Sensitive Tack of Adhesives Using an Inverted Probe Machine (ASTM D 2979-01; Reapproved 2009) using a probe tack tester PT-1000 (ChemInstruments). Prior to testing the samples were equilibrated 24 hours under controlled conditions at approx. room temperature (23±2° C.) and approx. 50% rh. For determining the tack, the tip of a cleaned probe with a diameter of 5 mm was brought into contact with the adhesive surface of the buprenorphine-containing layer structure for 1 second, at a defined rate (10±0.1 mm/s), under defined pressure (9.79±0.10 kPa), at a given temperature (23±2° C.) and the bond formed between probe and the adhesive was subsequently broken at the same rate. Tack was measured as the maximum force required, to break the adhesion bond (see ASTM D2979-01; Reapproved 2009). After finalization the mean value from the individual results of three associated samples were calculated and the mean tack value reported in [N].

TABLE 2.4
Tack [N] (n = 3)

| Ex. 1a | Ex. 1b | Comp. 1 |
|---|---|---|
| 1.48 | 2.28 | 1.19 |

### Measurement of Permeation

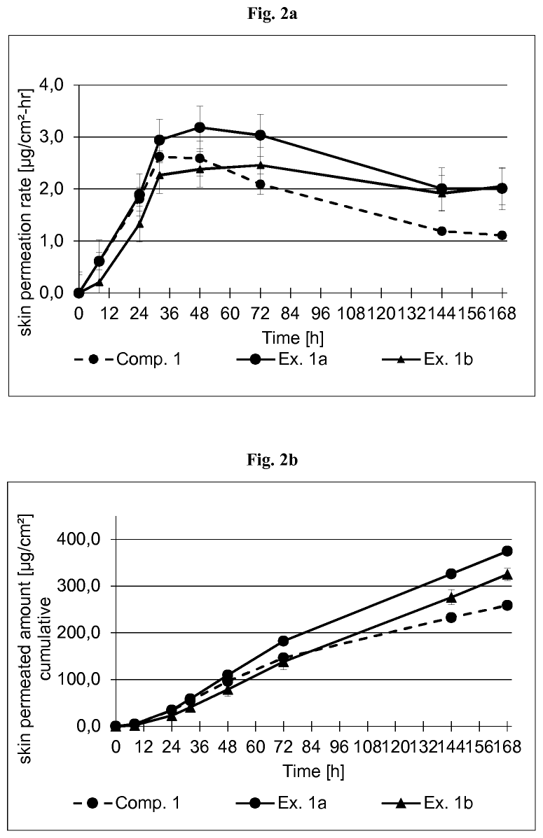

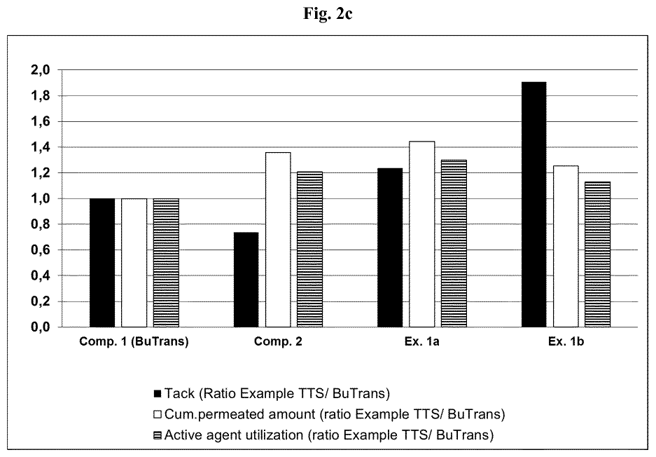

The permeated amount and the corresponding permeation rates of TTS prepared according to Examples 1a and 1b and Comparative Example 1 were determined by in vitro experiments in accordance with the OECD Guideline (adopted Apr. 13, 2004) carried out with a 9.0 ml Franz diffusion cell. Split thickness human skin from cosmetic surgeries (male abdomen, date of birth 1960) was used. A dermatome was used to prepare skin to a thickness of 800 μm, with an intact epidermis for all TTS. Due to the prolonged test (168 hours) 800 μm skin is used instead of the recommended 200 to 400 μm skin. Die cuts with an area of 1.188 cm² were punched from the TTS according to Comparative Example 1 and tested against 1.188 cm² die cuts of the TTS according to Ex. 1a and 1b. The concentrations of buprenorphine base in the receptor medium of the Franz diffusion cell (phosphate buffer solution pH 5.5 with 0.1% saline azide as antibacteriological agent) at a temperature of 32±1° C. were measured and the corresponding permeation rate calculated. The results for Examples 1a and 1b, as well as for Comparative Example 1, are shown in Tables 2.5 to 2.10, and FIGS. 2a and 2b.

TABLE 2.5
Permeated amount with SD [μg/cm²]

| Elapsed | Ex. 1a (n = 3) | | Ex. 1b (n = 3) | | Comp. 1 (n = 3) | |
|---|---|---|---|---|---|---|
| time [h] | Amount | SD | Amount | SD | Amount | SD |
| 0 | 0 | 0 | 0 | 0 | 0 | 0 |
| 8 | 4.89 | 0.62 | 1.67 | 1.11 | 4.90 | 1.32 |
| 24 | 30.17 | 1.94 | 21.23 | 7.75 | 28.90 | 3.60 |
| 32 | 23.50 | 0.87 | 18.10 | 3.06 | 20.97 | 0.93 |
| 48 | 50.93 | 1.89 | 38.07 | 3.33 | 41.40 | 5.34 |
| 72 | 72.77 | 4.88 | 59.00 | 2.38 | 50.10 | 4.68 |
| 144 | 144.00 | 1.73 | 138.00 | 3.46 | 85.83 | 4.37 |
| 168 | 48.17 | 1.65 | 49.13 | 4.96 | 26.63 | 0.76 |
| Cum. at 168 h | 374.4 | 8 | 325.2 | 13 | 258.7 | 10 |

TABLE 2.6
Permeation rate with SD [μg/cm²-h]

| Elapsed | Ex. 1a (n = 3) | | Ex. 1b (n = 3) | | Comp. 1 (n = 3) | |
|---|---|---|---|---|---|---|
| time [h] | Rate | SD | Rate | SD | Rate | SD |
| 0 | 0 | 0 | 0 | 0 | 0 | 0 |
| 8 | 0.61 | 0.08 | 0.21 | 0.14 | 0.61 | 0.17 |
| 24 | 1.89 | 0.12 | 1.33 | 0.48 | 1.81 | 0.23 |
| 32 | 2.94 | 0.11 | 2.26 | 0.38 | 2.62 | 0.12 |
| 48 | 3.18 | 0.12 | 2.38 | 0.21 | 2.59 | 0.33 |
| 72 | 3.03 | 0.20 | 2.46 | 0.10 | 2.09 | 0.20 |
| 144 | 2.00 | 0.02 | 1.92 | 0.05 | 1.19 | 0.06 |
| 168 | 2.01 | 0.07 | 2.05 | 0.21 | 1.11 | 0.03 |

TABLE 2.7
Cumulative permeation rate [μg/cm²-h]

| | Ex. 1a | Ex. 1b | Comp. 1 |
|---|---|---|---|
| over 72 hours | 2.5 | 1.9 | 2.0 |
| over 168 hours | 2.2 | 1.9 | 1.5 |
| Relative amendment | −12.0% | 0.9% | −24.2% |
| from hour 72 to hour | | | |
| 168 | | | |

TABLE 2.8
Ratio Example TTS/Comp. 1 (BuTrans ®) of
cumulative permeated amount after 168 hours of
release

| Ex. 1a | Ex. 1b | Comp. 1 |
|---|---|---|
| 1.4 | 1.3 | 1.0 |

TABLE 2.9
Ratio Cumulative permeated amount after 168
hours of release/API Loading (active agent
utilization)

| Ex. 1a | Ex. 1b | Comp. 1 |
|---|---|---|
| 0.42 | 0.36 | 0.32 |

TABLE 2.10
Ratio Example TTS/Comp. 1 (BuTrans ®) of active
agent utilization

| Ex. 1a | Ex. 1b | Comp. 1 |
|---|---|---|
| 1.3 | 1.1 | 1.0 |

### Comparative Example 3

The commercially available product Nicorette® is used as a reference TTS (Comp. 3). In particular, absolute mean values obtained from in vitro permeation studies (which may vary from study to study) can be compared by using Nicorette® as an internal standard. Nicorette® is a patch based on polyacrylates and contains nicotine in an amount of 1.75 mg/cm² (API loading).

### Comparative Example 4

The commercially available product Nicotinell® is used as a reference TTS (Comp. 4). The commercially available product Nicotinell® is a patch based on polyacrylates and contains nicotine in an amount of 1.75 mg/cm² (API loading). Die cuts with a nicotine content of 2.5 mg/cm² (API loading) used for the measurement of the permeation rate of Nicotinell® were obtained from the commercially available patch Nicotinell® by punching die cuts with an area of 1.188 cm² from the central area of the patch.

### Example 2A, 2B

### Coating Composition

The formulations of the nicotine-containing coating compositions of Examples 2a and 2b and the formulations of the active agent-free coating composition for the skin contact layers of Examples 2a and 2b are summarized in Tables 3.1 below. The formulations are based on weight percent.

TABLE 3.1

| | Ex. 2a and Ex. 2b | |
|---|---|---|
| | API containing composition | |
| Ingredient (Trade Name) | Amt [g] | Solids [%] |
| Nicotine base | 1.65 | 8.0 |
| Silicone acrylic hybrid PSA in | 19.02* | 92.0 |
| ethyl acetate. | | |
| (SilAc-PSA 7-6102 from Dow- | | |
| Corning Healthcare) | | |
| n-hexane | 10.13 | — |
| Total | 30.8 | 100.0 |
| | API free coating composition | |
| | for the skin contact layer | |
| | Solids [%] | |
| | Ex. 2a | Ex. 2b |
| Silicone acrylic hybrid PSA in n-heptane. | 100.0 | — |
| Solids content of 50% by weight (SilAc- | | |
| PSA 7-6101 from Dow-Corning | | |
| Healthcare) | | |
| Silicone acrylic hybrid PSA in | — | 100.0 |
| ethyl acetate. | | |
| Solids content of 50% by weight (SilAc- | | |
| PSA 7-6102 from Dow-Corning | | |
| Healthcare) | | |

*Amount given as solids amount as measured after evaporation of solvent

### Preparation of the API Coating Composition

A beaker was loaded with the silicone acrylic hybrid pressure-sensitive adhesive in the form of a mixture in ethyl acetate and the solvent evaporated. The silicone acrylic hybrid pressure-sensitive adhesive was re-dissolved in n-hexane, the nicotine base was added and the mixture was then stirred until a homogeneous mixture was obtained (stirring time is 3 hours or longer for re-dissolving the silicone acrylic hybrid pressure-sensitive adhesive throughout the examples, if not indicated otherwise).

### Coating of the API Coating Composition

The resulting nicotine-containing coating composition was coated on a polyethylene terephthalate backing layer (15 μm thickness) and dried for approx. 10 min at room temperature. The coating thickness gave an area weight of the nicotine-containing pressure sensitive adhesive layer of 113 g/m² (Ex. 2a) and 130 g/m² (Ex. 2b).

### Coating of the API Free Coating Composition (Skin Contact Layer) and Lamination

The active agent-free coating composition was coated on a polyethylene terephthalate film (fluorine polymer coated, 75 μm thickness, which may function as release liner, for Ex. 2a, or siliconized, 100 μm thickness, which may function as release liner, for Ex. 2b) and dried for approx. 10 min at room temperature, followed by 10 min at 80° C. The coating thickness gave an area weight of the skin contact layer of 115 g/m² (Ex. 2a) and 105 g/m² (Ex. 2b). This results in 100% by weight of silicone acrylic hybrid pressure-sensitive adhesive in this skin contact layer. The adhesive side of the nicotine-containing matrix layer that was laminated with a backing layer was laminated on the adhesive side of the coated and dried nicotine-free skin contact layer resulting in a nicotine-containing self-adhesive layer structure.

TABLE 3.2

| | Ex. 2a | Ex. 2b |
|---|---|---|
| Area Weight API containing matrix [g/m²] | 113 | 130 |
| Area Weight skin contact layer [g/m²] | 115 | 105 |
| API Loading [mg/cm²] | 0.9 | 1.04 |

### Preparation of the TTS

See Example 1.

### Measurement of Permeation

The permeated amount and the corresponding permeation rates of TTS prepared according to Examples 2a and 2b and Comparative Examples 3 and 4 were determined by in vitro experiments in accordance with the OECD Guideline (adopted Apr. 13, 2004) carried out with a 10 ml Franz diffusion cell.

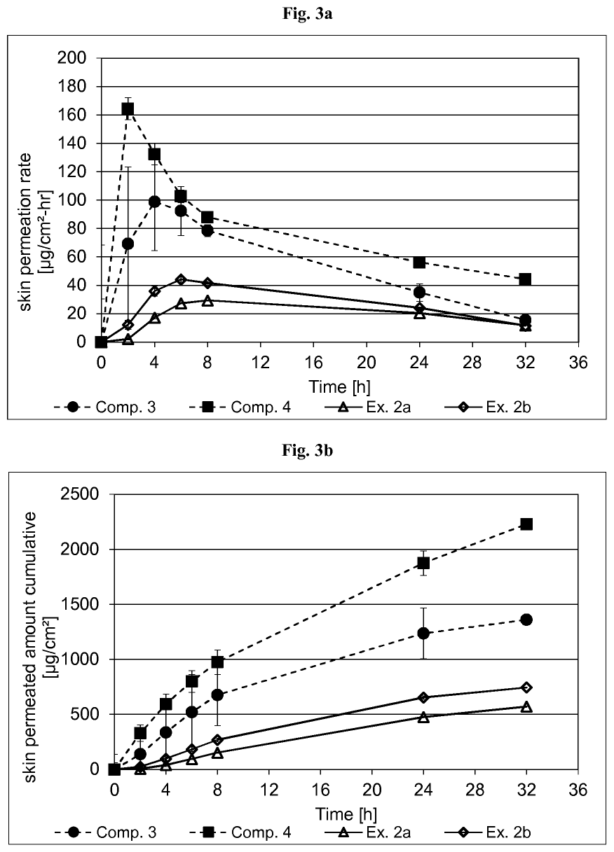

Split thickness human skin (female abdomen, date of birth 1970) was used. A dermatome was used to prepare skin to a thickness of 800 μm, with an intact epidermis for all TTS. Die cuts with an area of 1.188 cm² were punched from the TTS. The nicotine permeated amount in the receptor medium of the Franz cell (phosphate buffer solution pH 5.5 with 0.1% saline azide as antibacteriological agent) at a temperature of 32±1° C. was measured and the corresponding permeation rate calculated. The results are shown in Tables 3.3 to 3.7, and FIGS. 3a and 3b.

TABLE 3.3
Cumulative permeated amount with SD [μg/cm²]

| Elapsed | Ex. 2a (n = 3) | | Ex. 2b (n = 3) | | Comp. 3 (n = 3) | | Comp. 4 (n = 3) | |
|---|---|---|---|---|---|---|---|---|
| time [h] | Amount | SD | Amount | SD | Amount | SD | Amount | SD |
| 0 | 0 | 0 | 0 | 0 | 0 | 0 | 0 | 0 |
| 2 | 4.46 | 0.26 | 24.33 | 5.39 | 138.30 | 136.76 | 329.00 | 60.75 |
| 4 | 38.86 | 1.59 | 96.10 | 11.91 | 335.97 | 241.90 | 593.67 | 74.27 |
| 6 | 93.50 | 2.36 | 184.33 | 16.90 | 520.63 | 309.05 | 799.00 | 89.31 |
| 8 | 152.23 | 2.94 | 267.77 | 20.15 | 677.63 | 342.19 | 975.00 | 97.89 |
| 24 | 477.56 | 4.60 | 653.10 | 16.11 | 1236.30 | 279.90 | 1874.33 | 111.46 |
| 32 | 571.43 | 7.62 | 745.60 | 15.07 | 1360.47 | 231.02 | 2229.67 | 110.70 |

TABLE 3.4
Permeation rate with SD [μg/cm²-hr]

| Elapsed | Ex. 2a (n = 3) | | Ex. 2b (n = 3) | | Comp. 3 (n = 3) | | Comp. 4 (n = 3) | |
|---|---|---|---|---|---|---|---|---|
| time [h] | Rate | SD | Rate | SD | Rate | SD | Rate | SD |
| 0 | 0 | 0 | 0 | 0 | 0 | 0 | 0 | 0 |
| 2 | 2.23 | 0.13 | 12.17 | 2.70 | 69.15 | 68.38 | 164.50 | 30.38 |
| 4 | 17.20 | 0.72 | 35.88 | 3.28 | 98.83 | 54.21 | 132.33 | 7.75 |
| 6 | 27.32 | 0.77 | 44.12 | 3.59 | 92.33 | 34.32 | 102.67 | 7.52 |
| 8 | 29.37 | 0.43 | 41.72 | 1.78 | 78.50 | 17.30 | 88.00 | 4.44 |
| 24 | 20.33 | 0.34 | 24.08 | 0.96 | 34.92 | 4.12 | 56.21 | 1.08 |
| 32 | 11.73 | 0.38 | 11.56 | 0.62 | 15.52 | 6.16 | 44.42 | 0.38 |

TABLE 3.5
Cumulative permeation rate [μg/cm²-h]

| | Ex. 2a | Ex. 2b | Comp. 3 | Comp. 4 |
|---|---|---|---|---|
| | (n = 3) | (n = 3) | (n = 3) | (n = 3) |
| over 8 hours | 19.0 | 33.5 | 84.7 | 121.9 |
| over 24 hours | 19.9 | 27.2 | 51.5 | 78.1 |
| Relative amendment from | 4.6% | −18.7% | −39.2% | −35.9% |
| hour 8 to hour 24 | | | | |

TABLE 3.6
Ratio Cumulative permeated amount after 24 hours of
release/API Loading (active agent utilization)

| Ex. 2a | Ex. 2b | Comp. 3 | Comp. 4 |
|---|---|---|---|
| (n = 3) | (n = 3) | (n = 3) | (n = 3) |
| 0.53 | 0.63 | 0.71 | 0.75 |

TABLE 3.7
Ratio Example TTS/Comp. 3 (Nicorette ®) of active
agent utilization

| Ex. 2a | Ex. 2b | Comp. 3 | Comp. 4 |
|---|---|---|---|
| 0.75 | 0.89 | 1.00 | 1.06 |

### Comparative Example 5

The commercially available product Exelon® is used as a reference TTS (Comp. 5). In particular, absolute mean values obtained from in vitro permeation studies (which may vary from study to study) can be compared by using Exelon® as an internal standard. Exelon® is a commercially available rivastigmine-containing TTS product, having a rivastigmine-containing acrylic based layer (60 g/m²) and a rivastigmine-free silicone based skin contact layer (30 g/m²) and contains rivastigmine in an amount of 1.8 mg/cm² (API loading), provided by Novartis Pharma.

### Example 3A-D

### Coating Composition

The formulations of the rivastigmine-containing coating compositions of Examples 3a-d and the formulations of the active agent-free coating composition for the skin contact layers of Examples 3a-d are summarized in Tables 4.1 below. The formulations are based on weight percent.

TABLE 4.1

| Ingredient (Trade Name) | Ex. 3a | Ex. 3b | Ex. 3c | Ex. 3d |
|---|---|---|---|---|
| | API containing composition | | | |
| | Solids [%] | | | |
| Rivastigmine base | 20.0 | | | |
| Silicone acrylic hybrid PSA in n-heptane; | 80.0 | | | |
| Solids content of 50% by weight (SilAc- | | | | |
| PSA 7-6301 from Dow-Corning Healthcare) | | | | |
| Total | 100.0 | | | |
| | API free coating composition | | | |
| | for the skin contact layer | | | |
| | Solids [%] | | | |
| Silicone acrylic hybrid PSA in n-heptane; | 100.0 | — | — | — |
| Solids content of 50% by weight (SilAc- | | | | |
| PSA 7-6101 from Dow-Corning Healthcare) | | | | |
| Silicone acrylic hybrid PSA in ethyl acetate; | — | 100.0 | — | — |
| Solids content of 50% by weight (SilAc- | | | | |
| PSA 7-6102 from Dow-Corning Healthcare) | | | | |
| Silicone acrylic hybrid PSA in n-heptane; | — | — | 100.0 | — |
| Solids content of 50% by weight (SilAc- | | | | |
| PSA 7-6301 from Dow Corning Healthcare) | | | | |
| Silicone acrylic hybrid PSA in ethyl acetate; | — | — | — | 100.0 |
| Solids content of 50% by weight (SilAc- | | | | |
| PSA 7-6302 from Dow Corning Healthcare) | | | | |

### Preparation of the API Coating Composition

To adjust the viscosity of the silicone acrylic hybrid pressure-sensitive (SilAc-PSA 7-6301 from Dow Corning Healthcare) 199.25 g of the silicone acrylic hybrid pressure-sensitive adhesive with a solids content of about 50% was stirred in an open beaker with nitrogen exposure to a solids content of about 63.7% by weight. A beaker was then loaded with the silicone acrylic hybrid pressure-sensitive adhesive in the form of a mixture in n-heptane having a solids content of about 63.7% by weight and the rivastigmine base. The mixture was stirred at about 700 rpm until a homogenous mixture was obtained (at least 10 min) and degassed.

### Coating of the API Coating Composition

The resulting rivastigmine-containing mixture was coated within less than 24 h after the rivastigmine-containing mixture was finished on an adhesively equipped foil (fluorine polymer coated, 75 μm thickness, which may function as release liner) using a lab coater. The solvent was removed by drying in a first step at about room temperature (23±2° C.) for about 10 min, followed by a second drying step at about 70° C. for about 10 min.

The coating thickness was chosen such that removal of the solvents results in an area weight of the matrix layer of approx. 90 g/m². The dried film was then laminated with a backing layer (polyethylenterephthalate (PET) foil 23 μm; skin-colored).

### Coating of the API Free Coating Composition (Skin Contact Layer) and Lamination

The active agent-free coating composition was coated on a polyethylene terephthalate film (fluorine polymer coated, 75 μm thickness, which may function as release liner) and dried for approx. 10 min at room temperature, followed by approx. 10 min at 70° C.

The coating thickness was chosen such that removal of the solvents result in an area weight of the skin contact layer of approx. 30 g/m². This results in 100% by weight of silicone acrylic hybrid pressure-sensitive adhesive in this skin contact layer.

The adhesive side of the rivastigmine-containing matrix layer that was laminated with a backing layer was laminated, after removal of the release liner, on the adhesive side of the coated and dried rivastigmine-free skin contact layer resulting in a rivastigmine-containing self-adhesive layer structure.

TABLE 4.2

| | Ex. 3a | Ex. 3b | Ex. 3c | Ex. 3d |
|---|---|---|---|---|
| Area Weight API containing matrix [g/m²] | 90 | 90 | 90 | 90 |
| Area Weight skin contact layer [g/m²] | 30 | 30 | 30 | 30 |
| API Loading [mg/cm²] | 1.8 | 1.8 | 1.8 | 1.8 |

### Preparation of the TTS

See Example 1.

### Measurement of Permeation

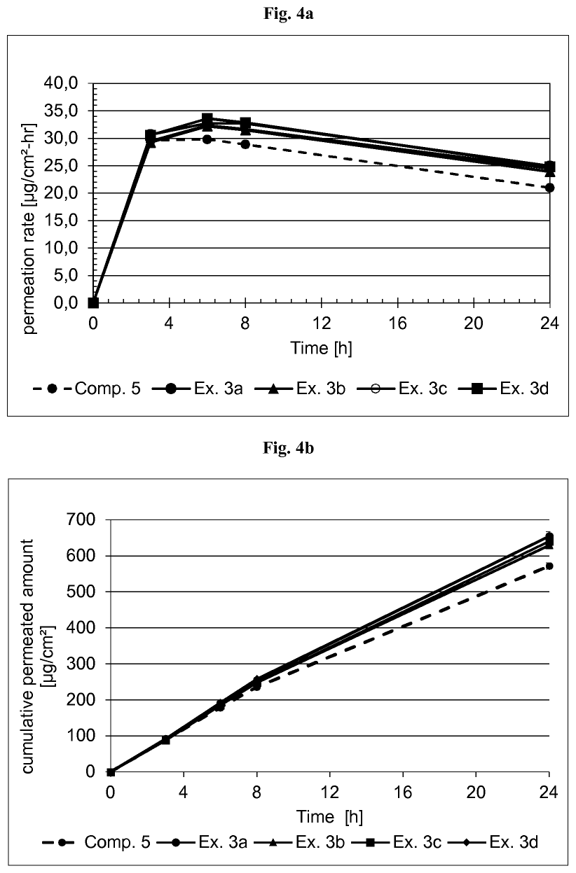

The permeated amount of TTS prepared according to Examples 3a-d and of Comparative Example 5 were determined by experiments in accordance with the EMA Guideline on quality of transdermal patches (adopted Oct. 23, 2014) carried out with a 10.0 ml Franz diffusion cell, wherein EVA-membrane (9% vinyl acetate; Scotchpak Cotran 9702 from 3M) having a thickness of 50 μm was used. Die cuts with an area of release of 1.188 cm² were punched from the TTS. The TTS was applied to the EVA-membrane by using an adhesive overlay. The rivastigmine permeated amount in the receptor medium of the Franz diffusion cell (phosphate buffer solution pH 5.5 with 0.1% sodium azide as antibacteriological agent) at a temperature of 32±1° C. was measured and the corresponding cumulative permeated amounts were calculated. The results are shown in Tables 4.3 to 4.7, and FIGS. 4a and 4b.

TABLE 4.3
Cumulative permeated amount with SD [μg/cm²]

| Elapsed | Ex. 3a (n = 3) | | Ex. 3b (n = 3) | | Ex. 3c (n = 3) | |
|---|---|---|---|---|---|---|
| time [h] | Amount | SD | Amount | SD | Amount | SD |
| 0 | 0 | 0 | 0 | 0 | 0 | 0 |
| 3 | 92.0 | 2.4 | 87.6 | 1.1 | 88.3 | 2.2 |
| 6 | 190.3 | 3.8 | 184.2 | 2.0 | 185.4 | 2.1 |
| 8 | 255.7 | 4.4 | 247.2 | 2.5 | 248.7 | 2.7 |
| 24 | 654.7 | 3.4 | 629.2 | 8.2 | 640.7 | 6.5 |

| | |
|---|---|
| Ex. 3d (n = 3) | Comp. 5 (n = 3) |

| | Amount | SD | Amount | SD |
|---|---|---|---|---|
| 0 | 0 | 0 | 0 | 0 |
| 3 | 91.7 | 1.6 | 88.7 | 1.0 |
| 6 | 192.7 | 1.0 | 178.1 | 2.8 |
| 8 | 258.3 | 1.1 | 235.9 | 4.2 |
| 24 | 655.3 | 12.6 | 571.9 | 8.7 |

TABLE 4.4
Permeation rate with SD [μg/cm²-hr]

| Elapsed | Ex. 3a (n = 3) | | Ex. 3b (n = 3) | | Ex. 3c (n = 3) | |
|---|---|---|---|---|---|---|
| time [h] | Amount | SD | Amount | SD | Amount | SD |
| 0 | 0 | 0 | 0 | 0 | 0 | 0 |
| 3 | 30.7 | 0.8 | 29.2 | 0.4 | 29.4 | 0.7 |
| 6 | 32.8 | 0.5 | 32.2 | 0.3 | 32.4 | 0.1 |
| 8 | 32.7 | 0.4 | 31.5 | 0.3 | 31.7 | 0.3 |
| 24 | 24.9 | 0.3 | 23.9 | 0.7 | 24.5 | 0.3 |

| | |
|---|---|
| Ex. 3d (n = 3) | Comp. 5 (n = 3) |

| | Amount | SD | Amount | SD |
|---|---|---|---|---|
| 0 | 0 | 0 | 0 | 0 |
| 3 | 30.6 | 0.5 | 29.6 | 0.3 |
| 6 | 33.7 | 0.4 | 29.8 | 0.6 |
| 8 | 32.8 | 0.5 | 28.9 | 0.7 |
| 24 | 24.8 | 0.7 | 21.0 | 0. |

TABLE 4.5
Cumulative permeation rate [μg/cm²-h]

| | Ex. 3a | Ex. 3b | Ex. 3c | Ex. 3d | Comp. 5 |
|---|---|---|---|---|---|
| over 8 hours | 32.0 | 30.9 | 31.1 | 32.3 | 29.5 |
| over 24 hours | 27.3 | 26.2 | 26.7 | 27.3 | 23.8 |
| Relative amendment from | −14.7% | −15.2% | −14.1% | −15.4% | −19.2% |
| hour 8 to hour 24 | | | | | |

TABLE 4.6
Ratio Cumulative permeated amount after 24 hours
of release/API Loading (active agent utilization)

| Ex. 3a | Ex. 3b | Ex. 3c | Ex. 3d | Comp. 5 |
|---|---|---|---|---|
| (n = 3) | (n = 3) | (n = 3) | (n = 3) | (n = 3) |
| 0.36 | 0.35 | 0.36 | 0.36 | 0.32 |

TABLE 4.7
Ratio Example TTS/Comp. 5 (Exelon ®)
of active agent utilization

| Ex. 3a | Ex. 3b | Ex. 3c | Ex. 3d | Comp. 5 |
|---|---|---|---|---|
| (n = 3) | (n = 3) | (n = 3) | (n = 3) | (n = 3) |
| 1.1 | 1.1 | 1.1 | 1.1 | 1.0 |

### The Invention Relates in Particular to the Following Further Items

1. A transdermal therapeutic system for the transdermal administration of an active agent comprising an active agent-containing layer structure,

the active agent-containing layer structure comprising:

A) a backing layer;

B) an active agent-containing layer,
wherein the active agent-containing layer comprises
a) a therapeutically effective amount of the active agent, and
b) at least one silicone acrylic hybrid polymer;
and

C) a skin contact layer,
wherein the skin contact layer comprises at least one silicone acrylic hybrid polymer.
   2. The transdermal therapeutic system according to item 1,
   wherein the skin contact layer in contact with the active agent-containing layer.
   3. The transdermal therapeutic system according to items 1 or 2,
   wherein the active agent-containing layer is an active agent-containing matrix layer.
   4. The transdermal therapeutic system according to any one of items 1 to 3,
   wherein the active agent-containing layer contains the silicone acrylic hybrid polymer in an amount of from about 20% to about 98% by weight based on the active agent-containing layer.
   5. The transdermal therapeutic system according to any one of items 1 to 4,
   wherein the active agent-containing layer contains the silicone acrylic hybrid polymer in an amount of from about 30% to about 95% by weight based on the active agent-containing layer.
   6. The transdermal therapeutic system according to any one of items 1 to 5,
   wherein the active agent-containing layer contains the silicone acrylic hybrid polymer in an amount of from about 50% to about 95% by weight based on the active agent-containing layer.
   7. The transdermal therapeutic system according to any one of items 1 to 6,
   wherein the skin contact layer contains the silicone acrylic hybrid polymer in an amount of from about 30% to about 100% by weight based on the skin contact layer.
   8. The transdermal therapeutic system according to any one of items 1 to 7,
   wherein the skin contact layer contains the silicone acrylic hybrid polymer in an amount of from about 50% to about 100% by weight based on the skin contact layer.
   9. The transdermal therapeutic system according to any one of items 1 to 8,
   wherein the skin contact layer contains the silicone acrylic hybrid polymer in an amount of from about 80% to about 100% by weight based on the skin contact layer.
   10. The transdermal therapeutic system according to any one of items 1 to 9,
   wherein the at least one silicone acrylic hybrid polymer contained in the active agent-containing layer is the same as the at least one silicone acrylic hybrid polymer contained in the skin contact layer.
   11. The transdermal therapeutic system according to any one of items 1 to 10,
   wherein the at least one silicone acrylic hybrid polymer contained in the active agent-containing layer is different compared to the at least one silicone acrylic hybrid polymer contained in the skin contact layer.
   12. The transdermal therapeutic system according to any one of items 1 to 11,
   wherein the silicone acrylic hybrid polymer in the skin contact layer contains a continuous, silicone external phase and a discontinuous, acrylic internal phase.
   13. The transdermal therapeutic system according to any one of items 1 to 11,
   wherein the silicone acrylic hybrid polymer in the skin contact layer contains a continuous, acrylic external phase and a discontinuous, silicone internal phase.
   14. The transdermal therapeutic system according to any one of items 1 to 13,
   wherein the silicone acrylic hybrid polymer in the active agent-containing layer contains a continuous, silicone external phase and a discontinuous, acrylic internal phase.
   15. The transdermal therapeutic system according to any one of items 1 to 13,
   wherein the silicone acrylic hybrid polymer in the active agent-containing layer contains a continuous, acrylic external phase and a discontinuous, silicone internal phase.
   16. The transdermal therapeutic system according to any one of items 1 to 15,
   wherein the active agent-containing layer has a continuous, silicone external phase and a discontinuous, acrylic internal phase.
   17. The transdermal therapeutic system according to any one of items 1 to 15,
   wherein the active agent-containing layer has a continuous, acrylic external phase and a discontinuous, silicone internal phase.
   18. The transdermal therapeutic system according to any one of items 1 to 17,
   wherein the skin contact layer has a continuous, silicone external phase and a discontinuous, acrylic internal phase.
   19. The transdermal therapeutic system according to any one of items 1 to 17,
   wherein the skin contact layer has a continuous, acrylic external phase and a discontinuous, silicone internal phase.
   20. The transdermal therapeutic system according to any one of items 1 to 19,
   wherein the at least one silicone acrylic hybrid polymer is a silicone acrylic hybrid pressure-sensitive adhesive.
   21. The transdermal therapeutic system according to item 20,
   wherein the at least one silicone acrylic hybrid pressure-sensitive adhesive has a weight ratio of silicone to acrylate of from 5:95 to 95:5.
   22. The transdermal therapeutic system according to item 20 or 21,
   wherein the at least one silicone acrylic hybrid pressure-sensitive adhesive has a weight ratio of silicone to acrylate of from 40:60 to 60:40.
   23. The transdermal therapeutic system according to any one of items 20 to 22,
   wherein the at least one silicone acrylic hybrid pressure-sensitive adhesive has a weight ratio of silicone to acrylate of about 50:50.
   24. The transdermal therapeutic system according to any one of items 20 to 23,
   wherein the at least one silicone acrylic hybrid pressure-sensitive adhesive is characterized by a solution viscosity at 25° C. and about 50% solids content in ethyl acetate of more than about 400 cP, preferably as measured using a Brookfield RVT viscometer equipped with a spindle number 5 at 50 RPM.
   25. The transdermal therapeutic system according to any one of items 20 to 24,
   wherein the at least one silicone acrylic hybrid pressure-sensitive adhesive is characterized by a solution viscosity at 25° C. and about 50% solids content in ethyl acetate of from about 500 cP to about 3,500 cP, preferably as measured using a Brookfield RVT viscometer equipped with a spindle number 5 at 50 RPM.
   26. The transdermal therapeutic system according to any one of items 20 to 25,
   wherein the at least one silicone acrylic hybrid pressure-sensitive adhesive is characterized by a solution viscosity at 25° C. and about 50% solids content in ethyl acetate of from about 1,000 cP to about 3,000 cP, preferably as measured using a Brookfield RVT viscometer equipped with a spindle number 5 at 50 RPM.
   27. The transdermal therapeutic system according to any one of items 20 to 26,
   wherein the at least one silicone acrylic hybrid pressure-sensitive adhesive is characterized by a solution viscosity at 25° C. and about 50% solids content in ethyl acetate of from about 1,200 cP to about 1,800 cP, preferably as measured using a Brookfield RVT viscometer equipped with a spindle number 5 at 50 RPM.
   28. The transdermal therapeutic system according to any one of items 20 to 27,
   wherein the at least one silicone acrylic hybrid pressure-sensitive adhesive is characterized by a solution viscosity at 25° C. and about 50% solids content in ethyl acetate of about 1,500 cP, preferably as measured using a Brookfield RVT viscometer equipped with a spindle number 5 at 50 RPM.
   29. The transdermal therapeutic system according to any one of items 20 to 26,
   wherein the at least one silicone acrylic hybrid pressure-sensitive adhesive is characterized by a solution viscosity at 25° C. and about 50% solids content in ethyl acetate of from about 2,200 cP to about 2,800 cP, preferably as measured using a Brookfield RVT viscometer equipped with a spindle number 5 at 50 RPM.
   30. The transdermal therapeutic system according to item 29,
   wherein the at least one silicone acrylic hybrid pressure-sensitive adhesive is characterized by a solution viscosity at 25° C. and about 50% solids content in ethyl acetate of about 2,500 cP, preferably as measured using a Brookfield RVT viscometer equipped with a spindle number 5 at 50 RPM.
   31. The transdermal therapeutic system according to any one of items 20 to 30,
   wherein the at least one silicone acrylic hybrid pressure-sensitive adhesive is characterized by a complex viscosity at 0.1 rad/s at 30° C. of less than about 1.0e9 Poise, preferably as measured using a Rheometrics ARES rheometer, wherein the rheometer is equipped with 8 mm plates and the gap zeroed.
   32. The transdermal therapeutic system according to any one of items 20 to 31,
   wherein the at least one silicone acrylic hybrid pressure-sensitive adhesive is characterized by a complex viscosity at 0.1 rad/s at 30° C. of from about 1.0e5 Poise to about 9.0e8 Poise, preferably as measured using a Rheometrics ARES rheometer, wherein the rheometer is equipped with 8 mm plates and the gap zeroed.
   33. The transdermal therapeutic system according to any one of items 20 to 32,
   wherein the at least one silicone acrylic hybrid pressure-sensitive adhesive is characterized by a complex viscosity at 0.1 rad/s at 30° C. of from about 9.0e5 Poise to about 1.0e7 Poise, preferably as measured using a Rheometrics ARES rheometer, wherein the rheometer is equipped with 8 mm plates and the gap zeroed.
   34. The transdermal therapeutic system according to any one of items 20 to 33,
   wherein the at least one silicone acrylic hybrid pressure-sensitive adhesive is characterized by a complex viscosity at 0.1 rad/s at 30° C. of from about 9.0e5 Poise to about 7.0e6 Poise, preferably as measured using a Rheometrics ARES rheometer, wherein the rheometer is equipped with 8 mm plates and the gap zeroed.
   35. The transdermal therapeutic system according to any one of items 20 to 34,
   wherein the at least one silicone acrylic hybrid pressure-sensitive adhesive is characterized by a complex viscosity at 0.1 rad/s at 30° C. of about 4.0e6 Poise, preferably as measured using a Rheometrics ARES rheometer, wherein the rheometer is equipped with 8 mm plates and the gap zeroed.
   36. The transdermal therapeutic system according to any one of items 20 to 32,
   wherein the at least one silicone acrylic hybrid pressure-sensitive adhesive is characterized by a complex viscosity at 0.1 rad/s at 30° C. of from about 2.0e6 Poise to about 9.0e7 Poise, preferably as measured using a Rheometrics ARES rheometer, wherein the rheometer is equipped with 8 mm plates and the gap zeroed.
   37. The transdermal therapeutic system according to any one of items 20 to 32,
   wherein the at least one silicone acrylic hybrid pressure-sensitive adhesive is characterized by a complex viscosity at 0.1 rad/s at 30° C. of from about 8.0e6 Poise to about 9.0e7 Poise, preferably as measured using a Rheometrics ARES rheometer, wherein the rheometer is equipped with 8 mm plates and the gap zeroed.
   38. The transdermal therapeutic system according to item 37,
   wherein the at least one silicone acrylic hybrid pressure-sensitive adhesive is characterized by a complex viscosity at 0.1 rad/s at 30° C. of about 1.0e7 Poise, preferably as measured using a Rheometrics ARES rheometer, wherein the rheometer is equipped with 8 mm plates and the gap zeroed.
   39. The transdermal therapeutic system according to any one of items 1 to 38,
   wherein the transdermal therapeutic system comprises at least two silicone acrylic hybrid polymers selected from at least two of the silicone acrylic hybrid polymer groups:
silicone acrylic hybrid pressure-sensitive adhesives characterized by a solution viscosity at 25° C. and about 50% solids content in ethyl acetate of from about 1,200 cP to about 1,800 cP, preferably as measured using a Brookfield RVT viscometer equipped with a spindle number 5 at 50 RPM, and
silicone acrylic hybrid pressure-sensitive adhesives characterized by a solution viscosity at 25° C. and about 50% solids content in ethyl acetate of from about 2,200 cP to about 2,800 cP, preferably as measured using a Brookfield RVT viscometer equipped with a spindle number 5 at 50 RPM.
   40. The transdermal therapeutic system according to any one of items 1 to 39,
   wherein the transdermal therapeutic system comprises at least two silicone acrylic hybrid polymers selected from at least two of the silicone acrylic hybrid polymer groups:
silicone acrylic hybrid pressure-sensitive adhesives characterized by a complex viscosity at 0.1 rad/s at 30° C. of from about 9.0e5 Poise to about 7.0e6 Poise, preferably as measured using a Rheometrics ARES rheometer, wherein the rheometer is equipped with 8 mm plates and the gap zeroed, and
silicone acrylic hybrid pressure-sensitive adhesives characterized by a complex viscosity at 0.1 rad/s at 30° C. of from about 8.0e6 Poise to about 9.0e7 Poise, preferably as measured using a Rheometrics ARES rheometer, wherein the rheometer is equipped with 8 mm plates and the gap zeroed.
   41. The transdermal therapeutic system according to item 39 or 40,
   wherein the at least two silicone acrylic hybrid polymers are contained in different layers.
   42. The transdermal therapeutic system according to item 39 or 40,
   wherein the at least two silicone acrylic hybrid polymers are contained in the active agent-containing layer.
   43. The transdermal therapeutic system according to item 39 or 40,
   wherein the at least two silicone acrylic hybrid polymers are contained in the skin contact layer.
   44. The transdermal therapeutic system according to any one of items 1 to 43,
   wherein the silicone acrylic hybrid polymer is obtainable from
(a) a silicon-containing pressure-sensitive adhesive composition comprising acrylate or methacrylate functionality.
   45. The transdermal therapeutic system according to any one of items 1 to 44,
   wherein the silicone acrylic hybrid polymer is a silicone acrylic hybrid pressure-sensitive adhesive comprising the reaction product of
(a) a silicon-containing pressure-sensitive adhesive composition comprising acrylate or methacrylate functionality;
(b) an ethylenically unsaturated monomer; and
(c) an initiator.
   46. The transdermal therapeutic system according to item 44 or 45,
   wherein the silicon-containing pressure-sensitive adhesive composition comprising acrylate or methacrylate functionality is the condensation reaction product of
(a1) a silicone resin, and
(a2) a silicone polymer, and
(a3) a silicon-containing capping agent comprising acrylate or methacrylate functionality.
   47. The transdermal therapeutic system according to any one of items 44 to 46,
   wherein the silicon-containing pressure-sensitive adhesive composition comprising acrylate or methacrylate functionality is the condensation reaction product of
(a1) a silicone resin, and
(a2) a silicone polymer, and
(a3) a silicon-containing capping agent comprising acrylate or methacrylate functionality, wherein said silicon-containing capping agent is of the general formula XYR′_{b}SiZ_{3-b}, wherein X is a monovalent radical of the general formula AE, where E is —O— or —NH— and A is an acryl group or methacryl group, Y is a divalent alkylene radical having from 1 to 6 carbon atoms, R′ is a methyl or a phenyl radical, Z is a monovalent hydrolysable organic radical or halogen, and b is 0 or 1;
wherein the silicone resin and silicone polymer are reacted to form a pressure-sensitive adhesive, wherein the silicon-containing capping agent is introduced prior to, during, or after the silicone resin and silicone polymer are reacted,
and wherein the silicon-containing capping agent reacts with the pressure-sensitive adhesive after the silicone resin and silicone polymer have been condensation reacted to form the pressure-sensitive adhesive, or the silicon-containing capping agent reacts in situ with the silicone resin and silicone polymer.
   48. The transdermal therapeutic system according to any one of items 45 to 47,
   wherein the ethylenically unsaturated monomer is selected from the group consisting of aliphatic acrylates, aliphatic methacrylates, cycloaliphatic acrylates, cycloaliphatic methacrylates, and combinations thereof, each of said compounds having up to 20 carbon atoms in the alkyl radical.
   49. The transdermal therapeutic system according to any one of items 45 to 48,
   wherein the ethylenically unsaturated monomer is a combination of 2-ethylhexyl acrylate and methyl acrylate.
   50. The transdermal therapeutic system according to any one of items 45 to 49,
   wherein the ethylenically unsaturated monomer is a combination of 2-ethylhexyl acrylate and methyl acrylate in a ratio of from 40:60 to 70:30, preferably in a ratio of from 65:35 to 55:45 or of from 55:45 to 45:50.
   51. The transdermal therapeutic system according to any one of items 45 to 50,
   wherein the reaction product of
(a) the silicon-containing pressure-sensitive adhesive composition comprising acrylate or methacrylate functionality;
(b) the ethylenically unsaturated monomer; and
(c) the initiator
   contains a continuous, silicone external phase and a discontinuous, acrylic internal phase.
   52. The transdermal therapeutic system according to any one of items 45 to 50,
   wherein the reaction product of
(a) the silicon-containing pressure-sensitive adhesive composition comprising acrylate or methacrylate functionality;
(b) the ethylenically unsaturated monomer; and
(c) the initiator
   contains a continuous, acrylic external phase and a discontinuous, silicone internal phase.
   53. The transdermal therapeutic system according to any one of items 1 to 52,
   wherein the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.
   54. The transdermal therapeutic system according to any one of items 1 to 53,
   wherein the transdermal therapeutic system further comprises at least one non-hybrid polymer.
   55. The transdermal therapeutic system according to any one of items 1 to 54,
   wherein the active agent-containing layer further comprises at least one non-hybrid polymer.
   56. The transdermal therapeutic system according to any one of items 1 to 54,
   wherein the skin contact layer further comprises at least one non-hybrid polymer.
   57. The transdermal therapeutic system according to any one of items 1 to 54,
   wherein the active agent-containing layer and the skin contact layer further comprise at least one non-hybrid polymer.
   58. The transdermal therapeutic system according to any one of items 1 to 57,
   wherein the transdermal therapeutic system further comprises at least one non-hybrid polymer based on polysiloxanes, polyisobutylenes, styrene-isoprene-styrene block copolymers, acrylates, or mixtures thereof.
   59. The transdermal therapeutic system according to any one of items 54 to 58,
   wherein the at least one non-hybrid polymer is a polymer based on polysiloxanes, a polymer based on polyisobutylenes, a styrene-isoprene-styrene block copolymer, a polyacrylate, or a mixture thereof.
   60. The transdermal therapeutic system according to any one of items 54 to 59,
   wherein the at least one non-hybrid polymer is a non-hybrid pressure-sensitive adhesive.
   61. The transdermal therapeutic system according to any one of items 54 to 60,
   wherein the at least one non-hybrid polymer is a non-hybrid pressure-sensitive adhesive based on polysiloxanes, polyisobutylenes, styrene-isoprene-styrene block copolymers, acrylates, or mixtures thereof.
   62. The transdermal therapeutic system according to any one of items 54 to 61,
   wherein the at least one non-hybrid polymer is a non-hybrid pressure-sensitive adhesive based on polysiloxanes.
   63. The transdermal therapeutic system according to any one of items 54 to 62,
   wherein the at least one non-hybrid polymer is a non-hybrid pressure-sensitive adhesive based on polysiloxanes characterized by a solution viscosity at 25° C. and about 60% solids content in n-heptane of more than about 150 mPa s, preferably as measured using a Brookfield RVT viscometer equipped with a spindle number 5 at 50 RPM.
   64. The transdermal therapeutic system according to any one of items 54 to 63,
   wherein the at least one non-hybrid polymer is a non-hybrid pressure-sensitive adhesive based on polysiloxanes characterized by a solution viscosity at 25° C. and about 60% solids content in n-heptane of from about 200 mPa s to about 700 mPa s, preferably as measured using a Brookfield RVT viscometer equipped with a spindle number 5 at 50 RPM.
   65. The transdermal therapeutic system according to any one of items 54 to 64,
   wherein the at least one non-hybrid polymer is a non-hybrid pressure-sensitive adhesive based on polysiloxanes characterized by a solution viscosity at 25° C. and about 60% solids content in heptane of about 450 mPa s or of about 500 mPa s, preferably as measured using a Brookfield RVT viscometer equipped with a spindle number 5 at 50 RPM.
   66. The transdermal therapeutic system according to any one of items 54 to 65,
   wherein the at least one non-hybrid polymer is a non-hybrid pressure-sensitive adhesive based on polysiloxanes characterized by a complex viscosity at 0.01 rad/s at 30° C. of less than about 1×10⁹ Poise, preferably as measured using a Rheometrics ARES rheometer, wherein the rheometer is equipped with 8 mm plates and the gap zeroed.
   67. The transdermal therapeutic system according to any one of items 54 to 66,
   wherein the at least one non-hybrid polymer is a non-hybrid pressure-sensitive adhesive based on polysiloxanes characterized by a complex viscosity at 0.01 rad/s at 30° C. of from about 1×10⁵ to about 9×10⁸ Poise, preferably as measured using a Rheometrics ARES rheometer, wherein the rheometer is equipped with 8 mm plates and the gap zeroed.
   68. The transdermal therapeutic system according to any one of items 54 to 67,
   wherein the at least one non-hybrid polymer is a non-hybrid pressure-sensitive adhesive based on polysiloxanes characterized by a complex viscosity at 0.01 rad/s at 30° C. of 1×10⁸ Poise, preferably as measured using a Rheometrics ARES rheometer, wherein the rheometer is equipped with 8 mm plates and the gap zeroed.
   69. The transdermal therapeutic system according to any one of items 54 to 67,
   wherein the at least one non-hybrid polymer is a non-hybrid pressure-sensitive adhesive based on polysiloxanes characterized by a complex viscosity at 0.01 rad/s at 30° C. of 5×10⁶ Poise, preferably as measured using a Rheometrics ARES rheometer, wherein the rheometer is equipped with 8 mm plates and the gap zeroed.
   70. The transdermal therapeutic system according to any one of items 54 to 61,
   wherein the at least one non-hybrid polymer is a non-hybrid pressure-sensitive adhesive based on acrylates.
   71. The transdermal therapeutic system according to any one of items 54 to 61,
   wherein the at least one non-hybrid polymer is an acrylate-based pressure-sensitive adhesive based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate.
   72. The transdermal therapeutic system according to any one of items 54 to 61,
   wherein the at least one non-hybrid polymer is an acrylate-based pressure-sensitive adhesive based on monomers selected from two or more of acrylic acid, butylacrylate, 2-ethylhexylacrylate, glycidylmethacrylate, 2-hydroxyethylacrylate, methylacrylate, methylmethacrylate, t-octylacrylamide and vinylacetate.
   73. The transdermal therapeutic system according to item 1 or 72,
   wherein the active agent-containing layer is an active agent-containing biphasic matrix layer having an inner phase comprising the therapeutically effective amount of the active agent, and having an outer phase comprising the at least one silicone acrylic hybrid polymer, wherein the inner phase forms dispersed deposits in the outer phase.
   74. The transdermal therapeutic system according to item 1 or 73,
   wherein the active agent-containing layer is an active agent-containing biphasic matrix layer having an inner phase comprising the therapeutically effective amount of the active agent and a carboxylic acid, and having an outer phase comprising the at least one silicone acrylic hybrid polymer, wherein the inner phase forms dispersed deposits in the outer phase.
   75. The transdermal therapeutic system according to item 73 or 74,
   wherein the dispersed deposits have a maximum sphere size of from 5 μm to 65 μm.
   76. The transdermal therapeutic system according to item 74,
   wherein the therapeutically effective amount of the active agent is in solution in the carboxylic acid.
   77. The transdermal therapeutic system according to any one of items 1 to 76,
   wherein the active agent-containing layer further comprises a non-hybrid polymer and wherein the at least one silicone acrylic hybrid polymer and the at least one non-hybrid polymer are contained in the active agent-containing layer such that the active agent-containing layer comprises
a) a therapeutically effective amount of active agent,
b) at least one silicone acrylic hybrid polymer,
c) at least one non-hybrid polymer, and
d) optionally a carboxylic acid.
   78. The transdermal therapeutic system according to item 1 or 77,
   wherein the active agent-containing layer is an active agent-containing biphasic matrix layer having an inner phase comprising the therapeutically effective amount of the active agent, and having an outer phase comprising the at least one silicone acrylic hybrid polymer and the at least one non-hybrid polymer, wherein the inner phase forms dispersed deposits in the outer phase.
   79. The transdermal therapeutic system according to item 1 or 78,
   wherein the active agent-containing layer is an active agent-containing biphasic matrix layer having an inner phase comprising the therapeutically effective amount of the active agent and a carboxylic acid, and having an outer phase comprising the at least one silicone acrylic hybrid polymer and the at least one non-hybrid polymer, wherein the inner phase forms dispersed deposits in the outer phase.
   80. The transdermal therapeutic system according to any one of items 1 to 79,
   wherein the skin contact layer contains the silicone acrylic hybrid polymer in an amount of from about 80% to about 100% by weight based on the skin contact layer,
   wherein the silicone acrylic hybrid polymer is a silicone acrylic hybrid pressure-sensitive adhesive having a weight ratio of silicone to acrylate of from 40:60 to 60:40, and wherein the ethylenically unsaturated monomers forming the acrylate comprise 2-ethylhexyl acrylate and methyl acrylate in a ratio of from 65:35 to 55:45, preferably wherein the skin contact layer has a continuous, acrylic external phase and a discontinuous, silicone internal phase.
   81. The transdermal therapeutic system according to any one of items 1 to 80,
   wherein the skin contact layer contains the silicone acrylic hybrid polymer in an amount of from about 80% to about 100% by weight based on the skin contact layer,
   wherein the silicone acrylic hybrid polymer is a silicone acrylic hybrid pressure-sensitive adhesive having a weight ratio of silicone to acrylate of from 40:60 to 60:40, and wherein the silicone acrylic hybrid pressure-sensitive adhesive is characterized by a solution viscosity at 25° C. and about 50% solids content in ethyl acetate of from about 1,200 cP to about 1,800 cP, preferably as measured using a Brookfield RVT viscometer equipped with a spindle number 5 at 50 RPM, and/or is characterized by a complex viscosity at 0.1 rad/s at 30° C. of from about 9.0e5 Poise to about 7.0e6 Poise, preferably as measured using a Rheometrics ARES rheometer, wherein the rheometer is equipped with 8 mm plates and the gap zeroed, preferably wherein the skin contact layer has a continuous, acrylic external phase and a discontinuous, silicone internal phase.
   82. The transdermal therapeutic system according to any one of items 1 to 81,
   wherein the skin contact layer is free of active agent.
   83. The transdermal therapeutic system according to any one of items 1 to 82,
   wherein the skin contact layer also comprises an active agent.
   84. The transdermal therapeutic system according to any one of items 1 to 83,
   wherein the active agent is contained in an amount of from 2% to 40% by weight based on the active agent-containing layer.
   85. The transdermal therapeutic system according to any one of items 1 to 84,
   wherein the active agent is contained in an amount of from 3% to 40% by weight based on the active agent-containing layer.
   86. The transdermal therapeutic system according to any one of items 1 to 85,
   wherein the active agent is contained in an amount of from 5% to 35% by weight based on the active agent-containing layer.
   87. The transdermal therapeutic system according to any one of items 1 to 86,
   wherein the active agent-containing layer is obtainable by coating and drying an active agent-containing coating composition that comprises a therapeutically effective amount of the active agent and the at least one silicone acrylic hybrid polymer.
   88. The transdermal therapeutic system according to any one of items 1 to 87,
   wherein the active agent is present in the active agent-containing layer in the form of the free base.
   89. The transdermal therapeutic system according to any one of items 1 to 88,
   wherein the active agent-containing layer further comprises a carboxylic acid.
   90. The transdermal therapeutic system according to item 89,
   wherein the carboxylic acid is contained in an amount sufficient so that the therapeutically effective amount of the active agent is so lubilized therein.
   91. The transdermal therapeutic system according to items 89 or 90,
   wherein the carboxylic acid is contained in an amount of from 2% to 20% by weight based on the active agent-containing layer.
   92. The transdermal therapeutic system according to any one of items 89 to 91,
   wherein the carboxylic acid is contained in an amount of from 4% to 15% by weight based on the active agent-containing layer.
   93. The transdermal therapeutic system according to any one of items 89 to 92,
   wherein the carboxylic acid is contained in an amount of from 5% to 12% by weight based on the active agent-containing layer.
   94. The transdermal therapeutic system according to any one of items 89 to 93,
   wherein the carboxylic acid is selected from the group consisting of C₃ to C₂₄ carboxylic acids.
   95. The transdermal therapeutic system according to any one of items 89 to 94,
   wherein the carboxylic acid is selected from the group consisting of oleic acid, linoleic acid, linolenic acid, levulinic acid, and mixtures thereof.
   96. The transdermal therapeutic system according to any one of items 89 to 95,
   wherein the carboxylic acid is levulinic acid.
   97. The transdermal therapeutic system according to any one of items 89 to 96,
   wherein the active agent and the carboxylic acid are contained in different amounts by weight based on the active agent-containing layer.
   98. The transdermal therapeutic system according to any one of items 89 to 97,
   wherein the carboxylic acid and the active agent are contained in an amount ratio of from 0.3:1 to 5.1.
   99. The transdermal therapeutic system according to any one of items 89 to 98,
   wherein the carboxylic acid is contained in less amounts by weight than the active agent based on the active agent-containing layer.
   100. The transdermal therapeutic system according to any one of items 89 to 99,
   wherein the carboxylic acid is levulinic acid, and wherein the levulinic acid and the active agent are contained in an amount ratio of from 0.3:1 to 5:1.
   101. The transdermal therapeutic system according to any one of items 1 to 100,
   wherein the area weight of the active agent-containing layer ranges from 20 to 160 g/m².
   102. The transdermal therapeutic system according to any one of items 1 to 101,
   wherein the area weight of the active agent-containing layer ranges from 30 to 140 g/m².
   103. The transdermal therapeutic system according to any one of items 1 to 102,
   wherein the area weight of the active agent-containing layer ranges from 40 to 140 g/m².
   104. The transdermal therapeutic system according to any one of items 1 to 103,
   wherein the area weight of the active agent-containing layer ranges from more than 80 to 140 g/m².
   105. The transdermal therapeutic system according to any one of items 1 to 104,
   wherein the area weight of the skin contact layer ranges from 5 to 150 g/m².
   106. The transdermal therapeutic system according to any one of items 1 to 105,
   wherein the area weight of the skin contact layer ranges from 10 to 140 g/m².
   107. The transdermal therapeutic system according to any one of items 1 to 106,
   wherein the area weight of the skin contact layer ranges from 5 to 40 g/m².
   108. The transdermal therapeutic system according to any one of items 1 to 107,
   wherein the area weight of the skin contact layer ranges from 10 to 30 g/m².
   109. The transdermal therapeutic system according to any one of items 1 to 105,
   wherein the area weight of the skin contact layer ranges from 20 to 150 g/m².
   110. The transdermal therapeutic system according to any one of items 1 to 106,
   wherein the area weight of the skin contact layer ranges from 20 to 130 g/m².
   111. The transdermal therapeutic system according to any one of items 1 to 106,
   wherein the area weight of the skin contact layer ranges from 20 to 40 g/m².
   112. The transdermal therapeutic system according to any one of items 1 to 106,
   wherein the area weight of the skin contact layer ranges from 80 to 130 g/m².
   113. The transdermal therapeutic system according to any one of items 1 to 106,
   wherein the area weight of the skin contact layer ranges from 90 to 120 g/m².
   114. The transdermal therapeutic system according to any one of items 1 to 113,
   wherein the active agent-containing layer structure contains 0.3 mg/cm² to 3.0 mg/cm² active agent based on the active agent-containing layer.
   115. The transdermal therapeutic system according to any one of items 1 to 114,
   wherein the active agent-containing layer structure contains 0.5 mg/cm² to 1.6 mg/cm² active agent based on the active agent-containing layer.
   116. The transdermal therapeutic system according to any one of items 1 to 115,
   wherein the active agent-containing layer structure contains more than 0.6 mg/cm² to less than 1.2 mg/cm² active agent based on the active agent-containing layer.
   117. The transdermal therapeutic system according to any one of items 1 to 114,
   wherein the active agent-containing layer structure contains more than 0.6 mg/cm² to less than 1.8 mg/cm² active agent based on the active agent-containing layer.
   118. The transdermal therapeutic system according to any one of items 1 to 114,
   wherein the active agent-containing layer structure contains 1.3 mg/cm² to 2.2 mg/cm² active agent based on the active agent-containing layer.
   119. The transdermal therapeutic system according to any one of items 1 to 114,
   wherein the active agent-containing layer structure contains 1.2 mg/cm² to less than 1.8 mg/cm′ active agent based on the active agent-containing layer.
   120. The transdermal therapeutic system according to any one of items 1 to 119,
   wherein the amount of active agent contained in the transdermal therapeutic system ranges from about 2.5 mg to about 6.5 mg active agent and the size of the active agent-containing layer providing the area of release ranging from about 1 cm² to about 4.5 cm².
   121. The transdermal therapeutic system according to any one of items 1 to 119,
   wherein the amount of active agent contained in the transdermal therapeutic system ranges from about 6 mg to about 12 mg active agent and the size of the active agent-containing layer providing the area of release ranging from about 3 cm² to about 7 cm².
   122. The transdermal therapeutic system according to any one of items 1 to 119,
   wherein the amount of active agent contained in the transdermal therapeutic system ranges from about 10 mg to about 17 mg active agent and the size of the active agent-containing layer providing the area of release ranging from about 5.5 cm² to about 10 cm².
   123. The transdermal therapeutic system according to any one of items 1 to 119,
   wherein the amount of active agent contained in the transdermal therapeutic system ranges from about 14 mg to about 22 mg active agent and the size of the active agent-containing layer providing the area of release ranging from about 7 cm² to about 13 cm².
   124. The transdermal therapeutic system according to any one of items 1 to 119,
   wherein the amount of active agent contained in the transdermal therapeutic system ranges from about 21 mg to about 33 mg active agent and the size of the active agent-containing layer providing the area of release ranging from about 11 cm² to about 19 cm².
   125. The transdermal therapeutic system according to any one of items 1 to 119,
   wherein the amount of active agent contained in the transdermal therapeutic system ranges from about 29 mg to about 43 mg active agent and the size of the active agent-containing layer providing the area of release ranging from about 17 cm² to about 23 cm².
   126. The transdermal therapeutic system according to any one of items 1 to 119,
   wherein the amount of active agent contained in the transdermal therapeutic system ranges from about 2.5 mg to about 6.5 mg active agent and the size of the active agent-containing layer providing the area of release ranging from about 1 cm² to about 2.4 cm².
   127. The transdermal therapeutic system according to any one of items 1 to 119,
   wherein the amount of active agent contained in the transdermal therapeutic system ranges from about 6 mg to about 12 mg active agent and the size of the active agent-containing layer providing the area of release ranging from about 2.5 cm² to less than 5 cm².
   128. The transdermal therapeutic system according to any one of items 1 to 119,
   wherein the amount of active agent contained in the transdermal therapeutic system ranges from about 10 mg to about 17 mg active agent and the size of the active agent-containing layer providing the area of release ranging from about 4.5 cm² to less than 7.5 cm².
   129. The transdermal therapeutic system according to any one of items 1 to 119,
   wherein the amount of active agent contained in the transdermal therapeutic system ranges from about 14 mg to about 22 mg active agent and the size of the active agent-containing layer providing the area of release ranging from about 6.5 cm² to less than 10 cm².
   130. The transdermal therapeutic system according to any one of items 1 to 119,
   wherein the amount of active agent contained in the transdermal therapeutic system ranges from about 21 mg to about 33 mg active agent and the size of the active agent-containing layer providing the area of release ranging from about 10.5 cm² to less than 15 cm².
   131. The transdermal therapeutic system according to any one of items 1 to 119,
   wherein the amount of active agent contained in the transdermal therapeutic system ranges from about 29 mg to about 43 mg active agent and the size of the active agent-containing layer providing the area of release ranging from about 16 cm² to less than 20 cm².
   132. The transdermal therapeutic system according to any one of items 1 to 131,
   wherein the active agent-containing layer further comprises an auxiliary polymer.
   133. The transdermal therapeutic system according to item 132,
   wherein the auxiliary polymer is contained in an amount of from about 0.5% to about 30% by weight based on the active agent-containing layer.
   134. The transdermal therapeutic system according to item 133,
   wherein the auxiliary polymer is contained in an amount of from about 2% to about 25% by weight based on the active agent-containing layer.
   135. The transdermal therapeutic system according to any one of items 132 to 134,
   wherein said auxiliary polymer is selected from the group consisting of alkyl methacrylate copolymers, amino alkyl methacrylate copolymers, methacrylic acid copolymers, methacrylic ester copolymers, ammonioalkyl methacrylate copolymers, polyvinylpyrrolidones, vinylpyrrolidone-vinyl acetate copolymers, polyvinyl caprolactam-polyvinyl acetate-polyethylene glycol copolymer, and mixtures thereof.
   136. The transdermal therapeutic system according to item 132 or 133,
   wherein said auxiliary polymer is a polyvinylpyrrolidone, preferably contained in an amount of from about 0.5% to about 8% by weight based on the active agent-containing layer.
   137. The transdermal therapeutic system according to any one of items 1 to 136,
   wherein the active agent-containing layer structure provides a tack of from 0.6 N to 8.0 N, preferably determined in accordance with the Standard Test Method for Pressure-Sensitive Tack of Adhesives Using an Inverted Probe Machine (ASTM D 2979-01; Reapproved 2009), wherein the transdermal therapeutic system samples were equilibrated 24 hours under controlled conditions at approx. room temperature (23±2° C.) and approx. 50% rh (relative humidity) prior to testing.
   138. The transdermal therapeutic system according to any one of items 1 to 137,
   wherein the active agent-containing layer structure provides a tack of from more than 1.2 N to 6.0 N, preferably determined in accordance with the Standard Test Method for Pressure-Sensitive Tack of Adhesives Using an Inverted Probe Machine (ASTM D 2979-01; Reapproved 2009), wherein the transdermal therapeutic system samples were equilibrated 24 hours under controlled conditions at approx. room temperature (23±2° C.) and approx. 50% rh (relative humidity) prior to testing.
   139. The transdermal therapeutic system according to any one of items 1 to 138,
   wherein the active agent-containing layer structure provides an adhesion force of from about 2 N/25 mm to about 16 N/25 mm, preferably determined using a tensile strength testing machine with an aluminium testing plate and a pull angle of 90°, wherein the transdermal therapeutic system samples were equilibrated 24 hours under controlled conditions at approx. room temperature (23±2° C.) and approx. 50% rh (relative humidity) prior to testing and are cut into pieces with a fixed width of 25 mm.
   140. The transdermal therapeutic system according to any one of items 1 to 139,
   wherein the active agent-containing layer structure provides an adhesion force of from about 3.5 N/25 mm to about 15 N/25 mm, preferably determined using a tensile strength testing machine with an aluminium testing plate and a pull angle of 90°, wherein the transdermal therapeutic system samples were equilibrated 24 hours under controlled conditions at approx. room temperature (23±2° C.) and approx. 50% rh (relative humidity) prior to testing and are cut into pieces with a fixed width of 25 mm.
   141. The transdermal therapeutic system according to any one of items 1 to 140,
   wherein the active agent-containing layer structure provides an adhesion force of from about 4 N/25 mm to about 15 N/25 mm, preferably determined using a tensile strength testing machine with an aluminium testing plate and a pull angle of 90°, wherein the transdermal therapeutic system samples were equilibrated 24 hours under controlled conditions at approx. room temperature (23±2° C.) and approx. 50% rh (relative humidity) prior to testing and are cut into pieces with a fixed width of 25 mm.
   142. The transdermal therapeutic system according to any one of items 1 to 141,
   providing a permeation rate of the active agent when measured in a comparable test with a commercial active agent reference transdermal therapeutic system that is therapeutically effective.
   143. The transdermal therapeutic system according to any one of items 1 to 142,
   providing a permeation rate of the active agent when measured in a comparable test with a commercial active agent reference transdermal therapeutic system over 24 hours, 32 hours, 48, hours 72 hours, 84 hours, 96 hours, or 168 hours that is therapeutically effective.
   144. Transdermal therapeutic system according to any one of items 1 to 143,
   providing a permeation rate of the active agent measured in a Franz diffusion cell with dermatomed human skin with a thickness of 800 μm, with an intact epidermis, in accordance with the OECD Guideline (adopted Apr. 13, 2004) when a phosphate buffer solution pH 5.5 with 0.1% saline azide as antibacteriological agent is used at a temperature of 32±1° C. that is constant within 20% points over about the last two-thirds of the administration period.
   145. Transdermal therapeutic system according to any one of items 1 to 144,
   providing a permeation rate of the active agent measured in a Franz diffusion cell with dermatomed human skin with a thickness of 800 μm, with an intact epidermis, in accordance with the OECD Guideline (adopted Apr. 13, 2004) when a phosphate buffer solution pH 5.5 with 0.1% saline azide as antibacteriological agent is used at a temperature of 32±1° C. that is constant within 20% points from hour 8 hours to hour 24 or from hour 72 to hour 168.
   146. Transdermal therapeutic system according to any one of items 1 to 144,
   providing a permeation rate of the active agent measured in a Franz diffusion cell with dermatomed human skin with a thickness of 800 μm, with an intact epidermis, in accordance with the OECD Guideline (adopted Apr. 13, 2004) when a phosphate buffer solution pH 5.5 with 0.1% saline azide as antibacteriological agent is used at a temperature of 32±1° C. that is constant within 20% points over the last 4 days of a 7-day administration period.
   147. Transdermal therapeutic system according to any one of items 1 to 144,
   providing a permeation rate of the active agent measured in a Franz diffusion cell with dermatomed human skin with a thickness of 800 μm, with an intact epidermis, in accordance with the OECD Guideline (adopted Apr. 13, 2004) when a phosphate buffer solution pH 5.5 with 0.1% saline azide as antibacteriological agent is used at a temperature of 32±1° C. that is constant within 20% points over the last 16 hours of a 24-hour administration period.
   148. Transdermal therapeutic system according to any one of items 1 to 144,
   providing a permeation rate of the active agent measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide as antibacteriological agent is used at a temperature of 32±1° C. that is constant within 20% points over about the last two-thirds of the administration period.
   149. Transdermal therapeutic system according to item 148,
   providing a permeation rate of the active agent measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide as antibacteriological agent is used at a temperature of 32±1° C. that is constant within 20% points from hour 8 hours to hour 24 or from hour 72 to hour 168.
   150. Transdermal therapeutic system according to item 148,
   providing a permeation rate of the active agent measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide as antibacteriological agent is used at a temperature of 32±1° C. that is constant within 20% points over the last 4 days of a 7-day administration period.
   151. Transdermal therapeutic system according to item 148,
   providing a permeation rate of the active agent measured in a Franz diffusion cell with an EVA membrane with a thickness of 50 μm in accordance with the EMA Guideline on quality of transdermal patches (adopted Oct. 23, 2014) when a phosphate buffer solution pH 5.5 with 0.1% saline azide as antibacteriological agent is used at a temperature of 32±1° C. that is constant within 20% points over the last 16 hours of a 24-hour administration period.
   152. Transdermal therapeutic system according to any one of items 144 to 151,
   wherein the permeation rate of the active agent is constant within less than 19% points.
   153. Transdermal therapeutic system according to any one of items 144 to 152,
   wherein the permeation rate of the active agent is constant within less than 18% points.
   154. Transdermal therapeutic system according to any one of items 144 to 153,
   wherein the permeation rate of the active agent is constant within less than 17% points.
   155. The transdermal therapeutic system according to any one of items 1 to 154,
   for use in a method of treatment.
   156. The transdermal therapeutic system according to any one of items 1 to 154,
   for use in a method of treating pain.
   157. The transdermal therapeutic system according to any one of items 1 to 154,
   for use in a method of treating pain wherein the transdermal therapeutic system is applied to the skin of a patient for about 24 hours.
   158. The transdermal therapeutic system according to any one of items 1 to 154,
   for use in a method of treating pain wherein the transdermal therapeutic system is applied to the skin of a patient for more than 3 days, or for 3.5 days, 4 days, 5 days, or 6 days.
   159. The transdermal therapeutic system according to any one of items 1 to 154,
   for use in a method of treating pain wherein the transdermal therapeutic system is applied to the skin of a patient for 7 days.
   160. Use of a transdermal therapeutic system according to any one of items 1 to 154,
   for the manufacture of a medicament.
   161. Use of a transdermal therapeutic system according to any one of items 1 to 154,
   for the manufacture of a medicament for treating pain.
   162. Use of a transdermal therapeutic system according to any one of items 1 to 154,
   for the manufacture of a medicament for treating pain that is applied to the skin of a patient for about 24 hours.
   163. Use of a transdermal therapeutic system according to any one of items 1 to 154,
   for the manufacture of a medicament for treating pain that is applied to the skin of a patient for more than 3 days, or for 3.5 days, 4 days, 5 days, or 6 days.
   164. Use of a transdermal therapeutic system according to any one of items 1 to 154,
   for the manufacture of a medicament for treating pain that is applied to the skin of a patient for 7 days.
   165. A method of treatment by applying to the skin of a patient a transdermal therapeutic system according to any one of items 1 to 159.
   166. A method of treating pain by applying to the skin of a patient a transdermal therapeutic system according to any one of items 1 to 154.
   167. A method of treating pain by applying to the skin of a patient a transdermal therapeutic system according to any one of items 1 to 154 for about 24 hours.
   168. A method of treating pain by applying to the skin of a patient a transdermal therapeutic system according to any one of items 1 to 154 for more than 3 days, or for about 3.5 days, about 4 days, about 5 days, or about 6 days.
   169. A method of treating pain by applying to the skin of a patient a transdermal therapeutic system according to any one of items 1 to 154 for about 7 days.
   170. The transdermal therapeutic system according to any one of items 1 to 154,
   for use in a method of treating nicotine addiction, in a method of smoking cessation treatment, in a method of treating Parkinson's disease or in a method of treating Alzheimer's disease.
   171. The transdermal therapeutic system according any one of items 1 to 154,
   for use in a method of treating nicotine addiction, in a method of smoking cessation treatment, in a method of treating Parkinson's disease or in a method of treating Alzheimer's disease, wherein the transdermal therapeutic system is applied to the skin of a patient for more than 8 hours.
   172. The transdermal therapeutic system according to any one of items 1 to 154,
   for use in a method of treating nicotine addiction, in a method of smoking cessation treatment, in a method of treating Parkinson's disease or in a method of treating Alzheimer's disease, wherein the transdermal therapeutic system is applied to the skin of a patient for about 12 hours.
   173. The transdermal therapeutic system according to any one of items 1 to 154,
   for use in a method of treating nicotine addiction, in a method of smoking cessation treatment, in a method of treating Parkinson's disease or in a method of treating Alzheimer's disease, wherein the transdermal therapeutic system is applied to the skin of a patient for at least 24 hours.
   174. Use of a transdermal therapeutic system according to any one of items 1 to 154,
   for the manufacture of a medicament for treating nicotine addiction, Parkinson's disease or Alzheimer's disease.
   175. Use of a transdermal therapeutic system according to any one of items 1 to 154,
   for the manufacture of a medicament for treating nicotine addiction, Parkinson's disease or Alzheimer's disease that is applied to the skin of a patient for more than 8 hours.
   176. Use of a transdermal therapeutic system according to any one of items 1 to 154,
   for the manufacture of a medicament for treating nicotine addiction, Parkinson's disease or Alzheimer's disease that is applied to the skin of a patient for about 12 hours.
   177. Use of a transdermal therapeutic system according to any one of items 1 to 154,
   for the manufacture of a medicament for treating nicotine addiction, Parkinson's disease or Alzheimer's disease that is applied to the skin of a patient for at least 24 hours.
   178. A method of treating nicotine addiction, a method of smoking cessation treatment, a method of treating Parkinson's disease or a method of treating Alzheimer's disease by applying to the skin of a patient a transdermal therapeutic system according to any one of items 1 to 154.
   179. A method of treating smoking cessation treatment, a method of treating Parkinson's disease or a method of treating Alzheimer's disease by applying to the skin of a patient a transdermal therapeutic system according to any one of items 1 to 154 for more than 8 hours.
   180. A method of treating smoking cessation treatment, a method of treating Parkinson's disease or a method of treating Alzheimer's disease by applying to the skin of a patient a transdermal therapeutic system according to any one of items 1 to 154 for about 12 hours.
   181. A method of treating smoking cessation treatment, a method of treating Parkinson's disease or a method of treating Alzheimer's disease by applying to the skin of a patient a transdermal therapeutic system according to any one of items 1 to 154 for at least 24 hours.
   182. The transdermal therapeutic system according to any one of items 1 to 154,
   for use in a method of preventing, treating, or delaying of progression of Alzheimer's disease, dementia associated with Parkinson's disease, and/or symptoms of traumatic brain injury.
   183. The transdermal therapeutic system according to any one of items 1 to 154,
   for use in a method of treating mild to moderate dementia caused by Alzheimer's or Parkinson's disease.
   184. The transdermal therapeutic system according to any one of items 1 to 154,
   for use in a method of treating mild to moderate dementia caused by Alzheimer's or Parkinson's disease, wherein the transdermal therapeutic system is applied to the skin of a patient for at least 24 hours.
   185. The transdermal therapeutic system according to any one of items 1 to 154,
   for use in a method of treating mild to moderate dementia caused by Alzheimer's or Parkinson's disease, wherein the transdermal therapeutic system is applied to the skin of a patient for about 24 hours.
   186. Use of a transdermal therapeutic system according to any one of items 1 to 154,
   for the manufacture of a medicament for preventing, treating, or delaying of progression of Alzheimer's disease, dementia associated with Parkinson's disease, and/or symptoms of traumatic brain injury.
   187. Use of a transdermal therapeutic system according to any one of items 1 to 154,
   for the manufacture of a medicament for treating mild to moderate dementia caused by Alzheimer's or Parkinson's disease.
   188. Use of a transdermal therapeutic system according to any one of items 1 to 154,
   for the manufacture of a medicament for preventing, treating, or delaying of progression of Alzheimer's disease, dementia associated with Parkinson's disease, and/or symptoms of traumatic brain injury that is applied to the skin of a patient for at least 24 hours, preferably about 24 hours.
   189. Use of a transdermal therapeutic system according to any one of items 1 to 154,
   for the manufacture of a medicament for treating mild to moderate dementia caused by Alzheimer's or Parkinson's disease that is applied to the skin of a patient for at least 24 hours, preferably about 24 hours.
   190. A method of preventing, treating, or delaying of progression of Alzheimer's disease, dementia associated with Parkinson's disease, and/or symptoms of traumatic brain injury by applying to the skin of a patient a transdermal therapeutic system according to any one of items 1 to 154.
   191. A method of treating mild to moderate dementia caused by Alzheimer's or Parkinson's disease by applying to the skin of a patient a transdermal therapeutic system according to any one of items 1 to 154.
   192. A method of preventing, treating, or delaying of progression of Alzheimer's disease, dementia associated with Parkinson's disease, and/or symptoms of traumatic brain injury by applying to the skin of a patient a transdermal therapeutic system according to any one of items 1 to 154 for at least or about 24 hours.
   193. A method of treating mild to moderate dementia caused by Alzheimer's or Parkinson's disease by applying to the skin of a patient a transdermal therapeutic system according to any one of items 1 to 154 for at least or about 24 hours.
   194. The transdermal therapeutic system according to any one of items 1 to 169,
   wherein the active agent is buprenorphine.
   195. The transdermal therapeutic system according to any one of items 1 to 155 and 170 to 181,
   wherein the active agent is nicotine.
   196. The transdermal therapeutic system according to any one of items 1 to 193,
   wherein the active agent is not buprenorphine or nicotine.
   197. The transdermal therapeutic system according to any one of items 1 to 155 and 182 to 193,
   wherein the active agent is rivastigmine.
   198. A method of manufacture of a transdermal therapeutic system according to any one of items 1 to 197 comprising the steps of:
1) providing an active agent-containing coating composition comprising
   a) the active agent,
   b) optionally a solvent, and
2) coating the active agent-containing coating composition onto a film in an amount to provide the desired area weight,
3) drying the coated active agent-containing coating composition to provide the active agent-containing layer,
4) providing an additional skin contact layer by coating and drying an additional coating composition according to steps 2 and 3, wherein the film is a release liner,
5) laminating the adhesive side of the skin contact layer onto the adhesive side of the active agent-containing layer to provide an active agent-containing layer structure with the desired area of release,
6) punching the individual systems from the active agent-containing layer structure,
7) optionally adhering to the individual systems an active agent-free self-adhesive layer structure comprising also a backing layer and an active agent-free pressure-sensitive adhesive layer and which is larger than the individual systems of active agent-containing self-adhesive layer structure,
   wherein at least one silicone acrylic hybrid polymer composition is added to the active agent-containing coating composition in step 1 and at least one silicone acrylic hybrid polymer composition is added to the additional coating composition in step 4.
   199. The method of manufacture according to item 198,
   wherein the film in step 2) is a release liner,
   wherein the active agent-containing layer is laminated after step 3) to a backing layer, and
   wherein the release liner of step 2) is removed before step 5).
   200. The method of manufacture according to item 198 or 199,
   wherein the active agent-containing coating composition of step 1) further comprises a carboxylic acid.
   201. The method of manufacture according to item 198,
   wherein the film in step 2) is a backing layer.
   202. The method of manufacture according to any one of items 198 to 201,
   wherein the at least one silicone acrylic hybrid polymer in the active agent-containing coating composition in step 1) is different to the silicone acrylic hybrid polymer in the additional coating composition in step 4).
   203. The method of manufacture according to any one of items 198 to 201,
   wherein the at least one silicone acrylic hybrid polymer in the active agent-containing coating composition in step 1) is the same silicone acrylic hybrid polymer as in the additional coating composition in step 4).
   204. The method of manufacture according to any one of items 198 to 203,
   wherein at least one silicone acrylic hybrid polymer composition is a silicone acrylic hybrid pressure-sensitive adhesive in ethyl acetate or n-heptane.
   205. The method of manufacture according to any one of items 198 to 204,
   wherein in step 1) a non-hybrid pressure-sensitive adhesive based on polysiloxanes is added.
   206. The method of manufacture according to any one of items 198 to 205,
   wherein in step 1) a non-hybrid pressure-sensitive adhesive based on acrylates is added.
   207. A transdermal therapeutic system for the transdermal administration of active agent comprising an active agent-containing layer structure,
the active agent-containing layer structure comprising:
A) a backing layer;
B) an active agent-containing matrix layer;
   wherein the active agent-containing matrix layer comprises
   a) the active agent in an amount of from 5 to 35% by weight based on the active agent-containing matrix layer, and
   b) a silicone acrylic hybrid polymer in an amount of from about 30% to about 95% by weight based on the active agent-containing matrix layer;
   and
C) a skin contact layer on the active agent-containing matrix layer comprising at least one silicone acrylic hybrid polymer in an amount of from about 50% to about 100% by weight based on the skin contact layer, wherein the silicone acrylic hybrid polymer is a silicone acrylic hybrid pressure-sensitive adhesive having a weight ratio of silicone to acrylate of from 40:60 to 60:40, preferably wherein the ethylenically unsaturated monomers forming the acrylate comprise 2-ethylhexyl acrylate and methyl acrylate in a ratio of from 65:35 to 55:45.
   208. A transdermal therapeutic system for the transdermal administration of rivastigmine comprising a rivastigmine-containing layer structure,
the rivastigmine-containing layer structure comprising:
A) a backing layer;
B) a rivastigmine-containing matrix layer;
   wherein the rivastigmine-containing matrix layer comprises
   a) a therapeutically effective amount of rivastigmine and
   b) at least one silicone acrylic hybrid polymer;
   and
C) a skin contact layer on the rivastigmine-containing matrix layer comprising at least one silicone acrylic hybrid pressure-sensitive adhesive.

1. A transdermal therapeutic system for the transdermal administration of an active agent comprising an active agent-containing layer structure,
the active agent-containing layer structure comprising:
A) a backing layer;
B) an active agent-containing layer,
wherein the active agent-containing layer comprises
a) a therapeutically effective amount of the active agent, and
b) at least one silicone acrylic hybrid polymer;
and
C) a skin contact layer,
wherein the skin contact layer comprises at least one silicone acrylic hybrid polymer.

2. The transdermal therapeutic system according to claim 1,
wherein the active agent-containing layer is an active agent-containing matrix layer, preferably an active agent-containing pressure-sensitive adhesive layer.

3. The transdermal therapeutic system according to claim 1 or 2,
wherein the active agent-containing layer contains the silicone acrylic hybrid polymer in an amount of from about 20% to about 98%, preferably of from about 30% to about 95%, more preferably of from about 50% to about 95% by weight based on the active agent-containing layer.

4. The transdermal therapeutic system according to any one of claims 1 to 3,
wherein the skin contact layer contains the silicone acrylic hybrid polymer in an amount of from about 30% to about 100%, preferably of from about 50% to about 100%, more preferably of from about 80% to about 100% by weight based on the skin contact layer.

5. The transdermal therapeutic system according to claim any one of claims 1 to 4,
wherein the at least one silicone acrylic hybrid polymer contained in the active agent-containing layer is the same as the at least one silicone acrylic hybrid polymer contained in the skin contact layer, or
wherein the at least one silicone acrylic hybrid polymer contained in the active agent-containing layer is different compared to the at least one silicone acrylic hybrid polymer contained in the skin contact layer.

6. The transdermal therapeutic system according to any one of claims 1 to 5,
wherein the silicone acrylic hybrid polymer in the skin contact layer contains a continuous, silicone external phase and a discontinuous, acrylic internal phase, or contains a continuous, acrylic external phase and a discontinuous, silicone internal phase, and
wherein the silicone acrylic hybrid polymer in the active agent-containing layer contains a continuous, silicone external phase and a discontinuous, acrylic internal phase, or contains a continuous, acrylic external phase and a discontinuous, silicone internal phase.

7. The transdermal therapeutic system according to any one of claims 1 to 6,
wherein the at least one silicone acrylic hybrid polymer is a silicone acrylic hybrid pressure-sensitive adhesive, preferably having a weight ratio of silicone to acrylate of from 5:95 to 95:5 more preferably of from 40:60 to 60:40.

8. The transdermal therapeutic system according to claim 7,
wherein the at least one silicone acrylic hybrid pressure-sensitive adhesive is characterized by a solution viscosity at 25° C. and about 50% solids content in ethyl acetate of more than about 400 cP, preferably of from about 500 cP to about 3,500 cP, and/or
wherein the at least one silicone acrylic hybrid pressure-sensitive adhesive is characterized by a complex viscosity at 0.1 rad/s at 30° C. of less than about 1.0e9 Poise, preferably of from about 1.0e5 Poise to about 9.0e8 Poise.

9. The transdermal therapeutic system according to any one of claims 1 to 8,
wherein the transdermal therapeutic system comprises at least two silicone acrylic hybrid polymers selected from at least two of the silicone acrylic hybrid polymer groups:
silicone acrylic hybrid pressure-sensitive adhesives characterized by a solution viscosity at 25° C. and about 50% solids content in ethyl acetate of from about 1,200 cP to about 1,800 cP, and/or characterized by a complex viscosity at 0.1 rad/s at 30° C. of from about 9.0e5 Poise to about 7.0e6 Poise, and
silicone acrylic hybrid pressure-sensitive adhesives characterized by a solution viscosity at 25° C. and about 50% solids content in ethyl acetate of from about 2,200 cP to about 2,800 cP, and/or characterized by a complex viscosity at 0.1 rad/s at 30° C. of from about 8.0e6 Poise to about 9.0e7 Poise.

10. The transdermal therapeutic system according to claim 9,
wherein the at least two silicone acrylic hybrid polymers are contained in different layers, or
wherein the at least two silicone acrylic hybrid polymers are contained in the active agent-containing layer.

11. The transdermal therapeutic system according to any one of claims 1 to 10,
wherein the silicone acrylic hybrid polymer is a silicone acrylic hybrid pressure-sensitive adhesive comprising the reaction product of
(a) a silicon-containing pressure-sensitive adhesive composition comprising acrylate or methacrylate functionality;
(b) an ethylenically unsaturated monomer; and
(c) an initiator,
wherein preferably the silicon-containing pressure-sensitive adhesive composition comprising acrylate or methacrylate functionality is the condensation reaction product of
(a1) a silicone resin, and
(a2) a silicone polymer, and
(a3) a silicon-containing capping agent comprising acrylate or methacrylate functionality.

12. The transdermal therapeutic system according to claim 11,
wherein the ethylenically unsaturated monomer is selected from the group consisting of aliphatic acrylates, aliphatic methacrylates, cycloaliphatic acrylates, cycloaliphatic methacrylates, and combinations thereof, each of said compounds having up to 20 carbon atoms in the alkyl radical, preferably the ethylenically unsaturated monomer is a combination of 2-ethylhexyl acrylate and methyl acrylate in a ratio of from 40:60 to 70:30, preferably in a ratio of from 65:35 to 55:45 or of from 55:45 to 45:50.

13. The transdermal therapeutic system according to any one of claims 1 to 12,
wherein the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.

14. The transdermal therapeutic system according to any one of claims 1 to 13,
wherein the active agent-containing layer and/or the skin contact layer further comprises at least one non-hybrid polymer, preferably wherein the at least one non-hybrid polymer is a non-hybrid pressure-sensitive adhesive based on polysiloxanes, polyisobutylenes, styrene-isoprene-styrene block copolymers, acrylates, or mixtures thereof, more preferably the at least one non-hybrid polymer is a non-hybrid pressure-sensitive adhesive based on polysiloxanes or acrylates.

15. The transdermal therapeutic system according to any one of claims 1 to 14,
wherein the active agent is contained in an amount of from 2% to 40%, preferably of from 3% to 40%, more preferably of from 5% to 35% by weight based on the active agent-containing layer.

16. The transdermal therapeutic system according to any one of claims 1 to 15,
wherein the area weight of the active agent-containing layer ranges from 20 to 160 g/m², preferably from 40 to 140 g/m², and the area weight of the skin contact layer ranges from 5 to 150 g/m², preferably from 10 to 140 g/m².

17. The transdermal therapeutic system according to any one of claims 1 to 16,
wherein the active agent-containing layer further comprises an auxiliary polymer, preferably in an amount of from about 0.5% to about 30% by weight based on the active agent-containing layer.

18. The transdermal therapeutic system according to claim 17,
wherein said auxiliary polymer is selected from the group consisting of alkyl methacrylate copolymers, amino alkyl methacrylate copolymers, methacrylic acid copolymers, methacrylic ester copolymers, ammonioalkyl methacrylate copolymers, polyvinylpyrrolidones, vinylpyrrolidone-vinyl acetate copolymers, polyvinyl caprolactam-polyvinyl acetate-polyethylene glycol copolymer, and mixtures thereof.

19. The transdermal therapeutic system according to any one of claims 1 to 18,
wherein the active agent-containing layer structure provides a tack of from 0.6 N to 8.0 N, preferably of from more than 1.2 N to 6.0 N.

20. Transdermal therapeutic system according to any one of claims 1 to 19,
providing a permeation rate of the active agent measured in a Franz diffusion cell, when a phosphate buffer solution pH 5.5 with 0.1% saline azide as antibacteriological agent is used at a temperature of 32±1° C., that is constant within 20% points, preferably within less than 19% points, over about the last two-thirds of the administration period.

21. The transdermal therapeutic system according to any one of claims 1 to 20,
wherein the active agent is buprenorphine, nicotine, or rivastigmine.

22. The transdermal therapeutic system according to any one of claims 1 to 21,
for use in a method of treatment,
preferably for use in a method of treating pain, or
for use in a method of treating nicotine addiction, or in a method of smoking cessation treatment, or in a method of treating Parkinson's disease or in a method of treating Alzheimer's disease, or for use in a method of preventing, treating, or delaying of progression of Alzheimer's disease, dementia associated with Parkinson's disease, and/or symptoms of traumatic brain injury, or for use in a method of treating mild to moderate dementia caused by Alzheimer's or Parkinson's disease, preferably wherein the transdermal therapeutic system is applied to the skin of a patient for at least or about 24 hours, for about 84 hours, or for about 168 hours.

23. A method of treatment by applying to the skin of a patient a transdermal therapeutic system according to any one of claims 1 to 22, preferably for at least or about 24 hours, for about 84 hours, or for about 168 hours.

24. A method of manufacture of a transdermal therapeutic system according to any one of claims 1 to 22 comprising the steps of:
1) providing an active agent-containing coating composition comprising
a) the active agent,
b) optionally a solvent, and
2) coating the active agent-containing coating composition onto a film in an amount to provide the desired area weight,
3) drying the coated active agent-containing coating composition to provide the active agent-containing layer,
4) providing an additional skin contact layer by coating and drying an additional coating composition according to steps 2 and 3, wherein the film is a release liner,
5) laminating the adhesive side of the skin contact layer onto the adhesive side of the active agent-containing layer to provide an active agent-containing layer structure with the desired area of release,
6) punching the individual systems from the active agent-containing layer structure,
7) optionally adhering to the individual systems an active agent-free self-adhesive layer structure comprising also a backing layer and an active agent-free pressure-sensitive adhesive layer and which is larger than the individual systems of active agent-containing self-adhesive layer structure,
wherein at least one silicone acrylic hybrid polymer composition is added to the active agent-containing coating composition in step 1 and at least one silicone acrylic hybrid polymer composition is added to the additional coating composition in step 4.
